SEQUENCE LISTING (19 sequences)

SEQ ID NO: 1 - protein, 743 aa, Artificial [sequence omitted]

SEQ ID NO: 2 - protein, 502 aa, Artificial
  hTNALP 1-502
MISPFLVLAIGTCLTNSLVPEKEKDPKYWRDQAQETLKYALELQKLNTNVAKNVIMFLGDGMGVSTVTAARILKGQLHHNPGEETRLEMDKFPFVALSKTYNTNAQVPDSAGTATAYLCGVKANEGTVGVSAATERSRCNTTQGNEVTSILRWAKDAGKSVGIVTTTRVNHATPSAAYAHSADRDWYSDNEMPPEALSQGCKDIAYQLMHNIRDIDVIMGGGRKYMYPKNKTDVEYESDEKARGTRLDGLDLVDTWKSFKPRYKHSHFIWNRTELLTLDPHNVDYLLGLFEPGDMQYELNRNNVTDPSLSEMVVVAIQILRKNPKGFFLLVEGGRIDHGHHEGKAKQALHEAVEMDRAIGQAGSLTSSEDTLTVVTADHSHVFTFGGYTPRGNSIFGLAPMLSDTDKKPFTAILYGNGPGYKVVGGERENVSMVDYAHNNYQAQSAVPLRHETHGGEDVAVFSKGPMAHLLHGVHEQNYVPHVMAYAACIGANLGHCAPASS

SEQ ID NO: 3 - protein, 227 aa, Artificial
  IgG-1 Fc fragment
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 4 - protein, 726 aa, Artificial
  hsTNALP-Fc-d10 without signal peptide
LVPEKEKDPKYWRDQAQETLKYALELQKLNTNVAKNVIMFLGDGMGVSTVTAARILKGQLHHNPGEETRLEMDKFPFVALSKTYNTNAQVPDSAGTATAYLCGVKANEGTVGVSAATERSRCNTTQGNEVTSILRWAKDAGKSVGIVTTTRVNHATPSAAYAHSADRDWYSDNEMPPEALSQGCKDIAYQLMHNIRDIDVIMGGGRKYMYPKNKTDVEYESDEKARGTRLDGLDLVDTWKSFKPRYKHSHFIWNRTELLTLDPHNVDYLLGLFEPGDMQYELNRNNVTDPSLSEMVVVAIQILRKNPKGFFLLVEGGRIDHGHHEGKAKQALHEAVEMDRAIGQAGSLTSSEDTLTVVTADHSHVFTFGGYTPRGNSIFGLAPMLSDTDKKPFTAILYGNGPGYKVVGGERENVSMVDYAHNNYQAQSAVPLRHETHGGEDVAVFSKGPMAHLLHGVHEQNYVPHVMAYAACIGANLGHCAPASSLKDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKDIDDDDDDDDDD

SEQ ID NO: 5 - protein, 485 aa, Artificial
  hsTNALP (18-502)
LVPEKEKDPKYWRDQAQETLKYALELQKLNTNVAKNVIMFLGDGMGVSTVTAARILKGQLHHNPGEETRLEMDKFPFVALSKTYNTNAQVPDSAGTATAYLCGVKANEGTVGVSAATERSRCNTTQGNEVTSILRWAKDAGKSVGIVTTTRVNHATPSAAYAHSADRDWYSDNEMPPEALSQGCKDIAYQLMHNIRDIDVIMGGGRKYMYPKNKTDVEYESDEKARGTRLDGLDLVDTWKSFKPRYKHSHFIWNRTELLTLDPHNVDYLLGLFEPGDMQYELNRNNVTDPSLSEMVVVAIQILRKNPKGFFLLVEGGRIDHGHHEGKAKQALHEAVEMDRAIGQAGSLTSSEDTLTVVTADHSHVFTFGGYTPRGNSIFGLAPMLSDTDKKPFTAILYGNGPGYKVVGGERENVSMVDYAHNNYQAQSAVPLRHETHGGEDVAVFSKGPMAHLLHGVHEQNYVPHVMAYAACIGANLGHCAPASS

SEQ ID NO: 6 - protein, 524 aa, bos taurus
MISPFLLLAIGTCFASSLVPEKEKDPKYWRDQAQQTLKNALRLQTLNTNVAKNVIMFLGDGMGVSTVTAARILKGQLHHSPGEETKLEMDKFPYVALSKTYNTNAQVPDSAGTATAYLCGVKANEGTVGVSAATQRSQCNTTQGNEVTSILRWAKDAGKSVGIVTTTRVNHATPSASYAHSADRDWYSDNEMPPEALSQGCKDIAYQLMYNIKDIEVIMGGGRKYMFPKNRTDVEYELDEKARGTRLDGLNLIDIWKSFKPKHKHSHYVWNRTDLLALDPHSVDYLLGLFEPGDMQYELNRNNATDPSLSEMVEMAIRILNKNPKGFFLLVEGGRIDHGHHEGKAKQALHEAVEMDQAIGQAGAMTSVEDTLTVVTADHSHVFTFGGYTPRGNSIFGLAPMVSDTDKKPFTAILYGNGPGYKVVGGERENVSMVDYAHNNYQAQSAVPLRHETHGGEDVAVFAKGPMAHLLHGVQEQNYIPHVMAYAACIGANRDHCASASSSGSPSPGPLLLLLALLPLGSLF

SEQ ID NO: 7 - protein, 524 aa, felis catus
MISPFLVLAIGTCLTNSLVPEKEKDPKYWRDQAQQTLKNALRLQKLNTNVVKNVIMFLGDGMGVSTVTAARILKGQLHHNPGEETRLEMDKFPYVALSKTYNTNAQVPDSAGTATAYLCGVKANEGTVGVSAATQRTQCNTTQGNEVTSILRWAKDSGKSVGIVTTTRVNHATPSAAYAHSADRDWYSDNEMPPEALSQGCKDIAYQLMHNVRDIEVIMGGGRKYMFPKNRTDVEYEMDEKARGTRLDGLNLVDIWKSFKPRHKHSHYVWNRTELLTLDPYGVDYLLGLFEPGDMQYELNRNSTTDPSLSEMVEIAIKILSKNPKGFFLLVEGGRIDHGHHEGKAKQALHEAVEMDQAIGRAGAMTSVEDTLTIVTADHSHVFTFGGYTPRGNSIFGLAPMVSDTDKKPFTSILYGNGPGYKVVGGERENVSMVDYAHNNYQAQSAVPLRHETHGGEDVAVFAKGPMAHLLHGVHEQNYIPHVMAYAACIGANLDHCASASSAGGPSPGPLFLLLALPSLGILF

SEQ ID NO: 8 - protein, 524 aa, homo sapiens
MISPFLVLAIGTCLTNSLVPEKEKDPKYWRDQAQETLKYALELQKLNTNVAKNVIMFLGDGMGVSTVTAARILKGQLHHNPGEETRLEMDKFPFVALSKTYNTNAQVPDSAGTATAYLCGVKANEGTVGVSAATERSRCNTTQGNEVTSILRWAKDAGKSVGIVTTTRVNHATPSAAYAHSADRDWYSDNEMPPEALSQGCKDIAYQLMHNIRDIDVIMGGGRKYMYPKNKTDVEYESDEKARGTRLDGLDLVDTWKSFKPRYKHSHFIWNRTELLTLDPHNVDYLLGLFEPGDMQYELNRNNVTDPSLSEMVVVAIQILRKNPKGFFLLVEGGRIDHGHHEGKAKQALHEAVEMDRAIGQAGSLTSSEDTLTVVTADHSHVFTFGGYTPRGNSIFGLAPMLSDTDKKPFTAILYGNGPGYKVVGGERENVSMVDYAHNNYQAQSAVPLRHETHGGEDVAVFSKGPMAHLLHGVHEQNYVPHVMAYAACIGANLGHCAPASSAGSLAAGPLLLALALYPLSVLF

SEQ ID NO: 9 - protein, 524 aa, mus musculis
MISPFLVLAIGTCLTNSFVPEKERDPSYWRQQAQETLKNALKLQKLNTNVAKNVIMFLGDGMGVSTVTAARILKGQLHHNTGEETRLEMDKFPFVALSKTYNTNAQVPDSAGTATAYLCGVKANEGTVGVSAATERTRCNTTQGNEVTSILRWAKDAGKSVGIVTTTRVNHATPSAAYAHSADRDWYSDNEMPPEALSQGCKDIAYQLMHNIKDIDVIMGGGRKYMYPKNRTDVEYELDEKARGTRLDGLDLISIWKSFKPRHKHSHYVWNRTELLALDPSRVDYLLGLFEPGDMQYELNRNNLTDPSLSEMVEVALRILTKNLKGFFLLVEGGRIDHGHHEGKAKQALHEAVEMDQAIGKAGAMTSQKDTLTVVTADHSHVFTFGGYTPRGNSIFGLAPMVSDTDKKPFTAILYGNGPGYKVVDGERENVSMVDYAHNNYQAQSAVPLRHETHGGEDVAVFAKGPMAHLLHGVHEQNYIPHVMAYASCIGANLDHCAWAGSGSAPSPGALLLPLAVLSLPTLF

SEQ ID NO: 10 - protein, 524 aa, rattus norvegicus
MILPFLVLAIGTCLTNSFVPEKEKDPSYWRQQAQETLKNALKLQKLNTNVAKNIIMFLGDGMGVSTVTAARILKGQLHHNTGEETRLEMDKFPFVALSKTYNTNAQVPDSAGTATAYLCGVKANEGTVGVSAATERTRCNTTQGNEVTSILRWAKDAGKSVGIVTTTRVNHATPSAAYAHSADRDWYSDNEMPPEALSQGCKDIAYQLMHNIKDIDVIMGGGRKYMYPKNRTDVEYELDEKARGTRLDGLDLISIWKSFKPRHKHSHYVWNRTELLALDPSRVDYLLGLFEPGDMQYELNRNNLTDPSLSEMVEVALRILTKNPKGFFLLVEGGRIDHGHHEGKAKQALHEAVEMDEAIGKAGTMTSQKDTLTVVTADHSHVFTFGGYTPRGNSIFGLAPMVSDTDKKPFTAILYGNGPGYKVVDGERENVSMVDYAHNNYQAQSAVPLRHETHGGEDVAVFAKGPMAHLLHGVHEQNYIPHVMAYASCIGANLDHCAWASSASSPSPGALLLPLALFPLRTLF

SEQ ID NO: 11 - protein, 502 aa, Canis familiaris
EKDPKYWRDQAQQTLKYALRLQNLNTNVAKNVIMFLGDGMGVSTVTATRILKGQLHHNPGEETRLEMDKFPYVALSKTYNTNAQVPDSAGTATAYLCGVKANEGTVGVSAATQRTHCNTTQGNEVTSILRWAKDAGKSVGIVTTTRVNHATPSAAYAHSADRDWYSDNEMPPEALSQGCKDIAYQLMHNVKDIEVIMGGGRKYMFPKNRTDVEYEMDEKSTGARLDGLNLIDIWKNFKPRHKHSHYVWNRTELLALDPYTVDYLLGLFDPGDMQYELNRNNVTDPSLSEMVEIAIKILSKKPRGFFLLVEGGRIDHGHHEGKAKQALHEAVEMDRAIGKAGVMTSLEDTLTVVTADHSHVFTFGGYTPRGNSIFGLAPMVSDTDKKPFTAILYGNGPGYKVVGGERENVSMVDYAHNNYQAQSAVPLRHETHGGEDVAVFAKGPMAHLLHGVHEQNYIPHVMAYAACIGANQDHCASASSAGGPSPGPLLLLLALLPVGILF

SEQ ID NO: 12 - protein, 528 aa, homo sapiens
MQGPWVLLLLGLRLQLSLGVIPAEEENPAFWNRQAAEALDAAKKLQPIQKVAKNLILFLGDGLGVPTVTATRILKGQKNGKLGPETPLAMDRFPYLALSKTYNVDRQVPDSAATATAYLCGVKANFQTIGLSAAARFNQCNTTRGNEVISVMNRAKQAGKSVGVVTTTRVQHASPAGTYAHTVNRNWYSDADMPASARQEGCQDIATQLISNMDIDVILGGGRKYMFPMGTPDPEYPADASQNGIRLDGKNLVQEWLAKHQGAWYVWNRTELMQASLDQSVTHLMGLFEPGDTKYEIHRDPTLDPSLMEMTEAALRLLSRNPRGFYLFVEGGRIDHGHHEGVAYQALTEAVMFDDAIERAGQLTSEEDTLTLVTADHSHVFSFGGYTLRGSSIFGLAPSKAQDSKAYTSILYGNGPGYVFNSGVRPDVNESESGSPDYQQQAAVPLSSETHGGEDVAVFARGPQAHLVHGVQEQSFVAHVMAFAACLEPYTACDLAPPACTTDAAHPVAASLPLLAGTLLLLGASAAP

SEQ ID NO: 13 - protein, 532 aa, homo sapiens
MQGPWVLLLLGLRLQLSLGIIPVEEENPDFWNRQAAEALGAAKKLQPAQTAAKNLIIFLGDGMGVSTVTAARILKGQKKDKLGPETFLAMDRFPYVALSKTYSVDKHVPDSGATATAYLCGVKGNFQTIGLSAAARFNQCNTTRGNEVISVMNRAKKAGKSVGVVTTTRVQHASPAGAYAHTVNRNWYSDADVPASARQEGCQDIATQLISNMDIDVILGGGRKYMFPMGTPDPEYPDDYSQGGTRLDGKNLVQEWLAKHQGARYVWNRTELLQASLDPSVTHLMGLFEPGDMKYEIHRDSTLDPSLMEMTEAALLLLSRNPRGFFLFVEGGRIDHGHHESRAYRALTETIMFDDAIERAGQLTSEEDTLSLVTADHSHVFSFGGYPLRGSSIFGLAPGKARDRKAYTVLLYGNGPGYVLKDGARPDVTESESGSPEYRQQSAVPLDGETHAGEDVAVFARGPQAHLVHGVQEQTFIAHVMAFAACLEPYTACDLAPRAGTTDAAHPGPSVVPALLPLLAGTLLLLGTATAP

SEQ ID NO: 14 - protein, 535 aa, homo sapiens
MLGPCMLLLLLLLGLRLQLSLGIIPVEEENPDFWNREAAEALGAAKKLQPAQTAAKNLIIFLGDGMGVSTVTAARILKGQKKDKLGPEIPLAMDRFPYVALSKTYNVDKHVPDSGATATAYLCGVKGNFQTIGLSAAARFNQCNTTRGNEVISVMNRAKKAGKSVGVVTTTRVQHASPAGTYAHTVNRNWYSDADVPASARQEGCQDIATQLISNMDIDVILGGGRKYMFRMGTPDPEYPDDYSQGGTRLDGKNLVQEWLAKRQGARYVWNRTELMQASLDPSVTHLMGLFEPGDMKYEIHRDSTLDPSLMEMTEAALRLLSRNPRGFFLFVEGGRIDHGHHESRAYRALTETIMFDDAIERAGQLTSEEDTLSLVTADHSHVFSFGGYPLRGSSIFGLAPGKARDRKAYTVLLYGNGPGYVLKDGARPDVTESESGSPEYRQQSAVPLDEETHAGEDVAVFARGPQAHLVHGVQEQTFIAHVMAFAACLEPYTACDLAPPAGTTDAAHPGRSVVPALLPLLAGTLLLLETATAP

SEQ ID NO: 15 - protein, 541 aa, Artificial
  Consensus ALP TNALP from various mammalian species and human ALP isozymes PLAP, GCALP, IALP (with signal peptide and GPI anchor domain)
XXXXXXXXXXXXXXXXXXXXXXXXXXXEXXPXXWXXXAXXXLXXAXXLQXXXXXXXKNXIXFLGDGXGVXTVTAXRILKGQXXXXXGXEXXLXMDXFPXXALSKTYXXXXXVPDSXXTATAYLCGVKXNXXTXGXSAAXXXXXCNTTXGNEVXSXXXXAKXXGKSVGXVTTTRVXHAXPXXXYAHXXXRXWYSDXXXPXXAXXXGCXDIAXQLXXNXXDIXVIXGGGRKYMXXXXXXDXEYXXDXXXXGXRLDGXXLXXXWXXXXXXXXXXXXXWNRTXLXXXXXXXXVXXLXGLFXPGDXXYEXXRXXXXDPSLXEMXXXAXXXLXXXXXGFXLXVEGGRIDHGHHEXXAXXALXEXXXXDXAIXXAGXXTSXXDTLXXVTADHSHVFXFGGYXXRGXSIFGLAPXXXXXDXKXXTXXLYGNGPGYXXXXGXRXXVXXXXXXXXXYXXQXAVPLXXETHXGEDVAVFXXGPXAHLXHGVXEQXXXXHVMAXAXCXXXXXXXXXXXXAXXXXXXXXXXXXXXXXLXXXXXXXLXXXXXXXX

SEQ ID NO: 16 - protein, 524 aa, Artificial
  Consensus TNALP from various mammalian species (with signal peptide and GPI anchor domain)
MIXPFLXLAIGTCXXXSXVPEKEXDPXYWRXQAQXTLKXALXLQXLNTNVXKNXIMFLGDGMGVSTVTAXRILKGQLHHXXGEETXLEMDKFPXVALSKTYNTNAQVPDSAGTATAYLCGVKANEGTVGVSAATXRXXCNTTQGNEVTSILRWAKDXGKSVGIVTTTRVNHATPSAXYAHSADRDWYSDNEMPPEALSQGCKDIAYQLMXNXXDIXVIMGGGRKYMXPKNXTDVEYEXDEKXXGXRLDGLXLXXXWKXFKPXXKHSHXXWNRTXLLXLDPXXVDYLLGLFXPGDMQYELNRNXXTDPSLSEMVXXAXXILXKXXXGFFLLVEGGRIDHGHHEGKAKQALHEAVEMDXAIGXAGXXTSXXDTLTXVTADHSHVFTFGGYTPRGNSIFGLAPMXSDTDKKPFTXILYGNGPGYKVVXGERENVSMVDYAHNNYQAQSAVPLRHETHGGEDVAVFXKGPMAHLLHGVXEQNYXPHVMAYAXCIGANXXHCAXAXSXXXXXXGXLXLXLAXXXXXXLF

SEQ ID NO: 17 - DNA, 2232 bp, Artificial
  hsTNALP-FcD10
atggtttcaccattcttagtactggccattggcacctgccttactaactccttagtgccagagaaagagaaagaccccaagtactggcgagaccaagcgcaagagacactgaaatatgccctggagcttcagaagctcaacaccaacgtggctaagaatgtcatcatgttcctgggagatgggatgggtgtctccacagtgacggctgcccgcatcctcaagggtcagctccaccacaaccctggggaggagaccaggctggagatggacaagttccccttcgtggccctctccaagacgtacaacaccaatgcccaggtccctgacagcgccggcaccgccaccgcctacctgtgtggggtgaaggccaatgagggcaccgtgggggtaagcgcagccactgagcgttcccggtgcaacaccacccaggggaacgaggtcacctccatcctgcgctgggccaaggacgctgggaaatctgtgggcattgtgaccaccacgagagtgaaccatgccacccccagcgccgcctacgcccactcggctgaccgggactggtactcagacaacgagatgccccctgaggccttgagccagggctgtaaggacatcgcctaccagctcatgcataacatcagggacattgacgtgatcatggggggtggccggaaatacatgtaccccaagaataaaactgatgtggagtatgagagtgacgagaaagccaggggcacgaggctggacggcctggacctcgttgacacctggaagagcttcaaaccgagatacaagcactcccacttcatctggaaccgcacggaactcctgacccttgacccccacaatgtggactacctattgggtctcttcgagccaggggacatgcagtacgagctgaacaggaacaacgtgacggacccgtcactctccgagatggtggtggtggccatccagatcctgcggaagaaccccaaaggcttcttcttgctggtggaaggaggcagaattgaccacgggcaccatgaaggaaaagccaagcaggccctgcatgaggcggtggagatggaccgggccatcgggcaggcaggcagcttgacctcctcggaagacactctgaccgtggtcactgcggaccattcccacgtcttcacatttggtggatacaccccccgtggcaactctatctttggtctggcccccatgctgagtgacacagacaagaagcccttcactgccatcctgtatggcaatgggcctggctacaaggtggtgggcggtgaacgagagaatgtctccatggtggactatgctcacaacaactaccaggcgcagtctgctgtgcccctgcgccacgagacccacggcggggaggacgtggccgtcttctccaagggccccatggcgcacctgctgcacggcgtccacgagcagaactacgtcccccacgtgatggcgtatgcagcctgcatcggggccaacctcggccactgtgctcctgccagctcgcttaaggacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaagatatcgatgacgatgacgatgacgatgacgatgactag

SEQ ID NO: 18 - protein, 541 aa, Artificial
  Consensus ALP TNALP from various mammalian species and human ALP isozymes PLAP, GCALP, IALP (with signal peptide and GPI anchor domain)
XXXXXXXXXXXXXXXXXXXXXXXXXXXEXXPXXWXXXAXXXLXXAXXLQXXXXXXAKNXIXFLGDGMGVXTVTAXRILKGQXXXXXGXEXXLXMDXFPXXALSKTYXXXXXVPDSXXTATAYLCGVKXNXXTXGXSAAXXXXXCNTTXGNEVTSXXXXAKXXGKSVGXVTTTRVXHAXPXXXYAHXXXRXWYSDXXXPXXAXXXGCXDIAXQLXXNXXDIXVIXGGGRKYMXXXXXXDXEYXXDXXXXGXRLDGXXLXXXWXXXXXXXXXXXXXWNRTXLXXXXXXXXVXXLXGLFXPGDMXYEXXRXXXXDPSLXEMXXXAXXXLXXXXXGFXLXVEGGRIDHGHHEXXAXXALXEXXXXDXAIXXAGXXTSXXDTLXXVTADHSHVFXFGGYXXRGXSIFGLAPXXXXXDXKXXTXXLYGNGPGYXXXXGXRXXVXXXXXXXXXYXXQXAVPLXXETHXGEDVAVFXXGPXAHLXHGVXEQXXXXHVMAXAXCXXXXXXXXXXXXAXXXXXXXXXXXXXXXXLXXXXXXXLXXXXXXXX

SEQ ID NO: 19 - protein, 524 aa, Artificial
  Consensus TNALP from various mammalian species (with signal peptide and GPI anchor domain)
MIXPFLXLAIGTCXXXSXVPEKEXDPXYWRXQAQXTLKXALXLQXLNTNVAKNXIMFLGDGMGVSTVTAXRILKGQLHHXXGEETXLEMDKFPXVALSKTYNTNAQVPDSAGTATAYLCGVKANEGTVGVSAATXRXXCNTTQGNEVTSILRWAKDXGKSVGIVTTTRVNHATPSAXYAHSADRDWYSDNEMPPEALSQGCKDIAYQLMXNXXDIXVIMGGGRKYMXPKNXTDVEYEXDEKXXGXRLDGLXLXXXWKXFKPXXKHSHXXWNRTXLLXLDPXXVDYLLGLFXPGDMQYELNRNXXTDPSLSEMVXXAXXILXKXXXGFFLLVEGGRIDHGHHEGKAKQALHEAVEMDXAIGXAGXXTSXXDTLTXVTADHSHVFTFGGYTPRGNSIFGLAPMXSDTDKKPFTXILYGNGPGYKVVXGERENVSMVDYAHNNYQAQSAVPLRHETHGGEDVAVFXKGPMAHLLHGVXEQNYXPHVMAYAXCIGANXXHCAXAXSXXXXXXGXLXLXLAXXXXXXLF

(19) United States
(12) Patent Application Publication
(10) Pub. No.: US 2010/0297119 A1
(43) Pub. Date: Nov. 25, 2010

(54) BONE TARGETED ALKALINE PHOSPHATASE, KITS AND METHODS OF USE THEREOF

(75) Inventors: Philippe Crine, Outremont (CA); Guy Boileau, Brossard (CA); Thomas P. Loisel, Montreal (CA); Isabelle Lemire, Montreal (CA); Pierre Léonard, Montreal (CA); Robert Heft, Dollard-des-Ormeaux (CA); Hal Landy, Dover, MA (US)

Correspondence Address: CLARK & ELBING LLP, 101 FEDERAL STREET, BOSTON, MA, 02110, US

(73) Assignee: ENOBIA PHARMA INC.

(21) Appl. No.: 12/599,679

(22) PCT Filed: May 12, 2008
(86) PCT No.: PCT/CA08/00923

§ 371 Date: Jul. 19, 2010

Related U.S. Application Data
(60) Provisional application No. 60/917,589, filed on May 11, 2007.

(51) Int. Cl. A61K 39/395; C12N 9/16; A61K 38/46; C07H 21/00; C12N 15/85; C12N 5/10; A61P 19/08

(52) U.S. Cl. 424/134.1; 435/196; 424/94.6; 536/23.2; 435/320.1; 435/325

(57) ABSTRACT

A bone targeted alkaline phosphatase comprising a polypeptide having the structure: Z-sALP-Y-spacer-X-Wₙ-V, wherein sALP is the extracellular domain of the alkaline phosphatase; wherein V is absent or is an amino acid sequence of at least one amino acid; X is absent or is an amino acid sequence of at least one amino acid; Y is absent or is an amino acid sequence of at least one amino acid; Z is absent or is an amino acid sequence of at least one amino acid; and Wₙ is a polyaspartate or a polyglutamate wherein n=10 to 16. Kits and methods of use thereof.

### CROSS REFERENCE TO RELATED APPLICATIONS

This application claims priority on U.S. provisional application Ser. No. 60/917,589, filed on May 11, 2007. All documents above are incorporated herein in their entirety by reference.

### STATEMENT REGARDING FEDERALLY SPONSORED RESEARCH OR DEVELOPMENT

N/A.

### FIELD OF THE INVENTION

The present invention relates to bone targeted alkaline phosphatase, kits and methods of use thereof.

### BACKGROUND OF THE INVENTION

Hypophosphatasia (HPP) is a rare, heritable form of rickets or osteomalacia (Whyte 2001) with an incidence as great as 1 per 2,500 births in Canadian Mennonites (Greenberg, 1993) and of 1 per 100,000 births in the general population for the more severe form of the disease. Milder forms are more prevalent. This “inborn error of metabolism” is caused by loss-of-function mutation(s) in the gene (ALPL) that encodes the tissue-nonspecific isozyme of alkaline phosphatase (TNALP; a.k.a liver/bone/kidney type ALP) (Weiss et al. 1988; Henthorn et al. 1992a; Henthorn et al. 1992b; Zurutuza et al. 1999; Milián 1995). The biochemical hallmark is subnormal ALP activity in serum (hypophosphatasemia), which leads to elevated blood and/or urine levels of three phosphocompound substrates: inorganic pyrophosphate (PPᵢ) phosphoethanolamine (PEA), and pyridoxal 5′-phosphate (PLP) (Whyte 1994).

HPP features a remarkable range of severity ranging from (most severe to mildest) perinatal, infantile, childhood, adult, and odontohypophosphatasia forms, classified historically according to age at diagnosis (Whyte 2001). There may be almost complete absence of bone mineralization in utero with stillbirth, or spontaneous fractures and dental disease occurring first in adult life. Perinatal (lethal) Hypophosphatasia is expressed in utero and can cause stillbirth. Some neonates may survive several days but suffer increased respiratory compromise due to the hypoplastic and rachitic disease of the chest. In infantile HPP, diagnosed before 6 months-of-age, postnatal development seems normal until onset of poor feeding, inadequate weight gain, and appearance of rickets. Radiological features are characteristic and show impaired skeletal mineralization, sometimes with progressive skeletal demineralization leading to rib fractures and chest deformity. Childhood Hypophosphatasia has also highly variable clinical expression. Premature loss of deciduous teeth results from aplasia, hypoplasia or dysplasia of dental cementum that connects the tooth root with the periodontal ligament. Rickets causes short stature and the skeletal deformities may include bowed legs, enlargement of the wrists, knees and ankles as a result of flared metaphysis. Adult HPP usually presents during middle age, although frequently there is a history of rickets and/or early loss of teeth followed by good health during adolescence and young adult life. Recurrent metatarsal stress fractures are common and calcium pyrophosphate dihydrate deposition causes attacks of arthritis and pyrophosphate arthropathy. Odontohypophosphatasia is diagnosed when the only clinical abnormality is dental disease and radiological studies and even bone biopsies reveal no signs of rickets or osteomalacia.

The severe clinical forms of Hypophosphatasia are usually inherited as autosomal recessive traits with parents of such patients showing subnormal levels of serum AP activity (Whyte 2001). For the milder forms of hypophosphatasia, i.e., adult and odontohypophosphatasia, an autosomal dominant pattern of inheritance has also been documented (Whyte 2001).

In the healthy skeleton, TNALP is an ectoenzyme present on the surface of the plasma membrane of osteoblasts and chondrocytes, including on the membranes of their shed matrix vesicles (MVs) (Ali et al. 1970; Bernard 1978) where the enzyme is particularly enriched (Morris et al. 1992). Deposition of hydroxyapatite during bone mineralization normally initiates within the lumen of these MVs (Anderson et al. 2005a). Electron microscopy has shown that TNALP-deficient MVs from severely affected HPP patients and Akp2^{-/-} mice (a TNALP null mouse model, see below) contain hydroxyapatite crystals, but that extravesicular crystal propagation appears retarded (Anderson 1997; Anderson 2004). This defect is attributed to the extracellular accumulation of PPᵢ, a potent inhibitor of calcification (Meyer 1984) due to deficiency of TNALP activity (Hessle et al. 2002; Harmey et al. 2004; Harmey et al. 2006.).

When PPi is present at near physiological concentrations, in the range of 0.01-0.1 mM, PPi has the ability to stimulate mineralization in organ-cultured chick femurs (Anderson & Reynolds 1973) and also by isolated rat MVs (Anderson et al. 2005b), while at concentrations above 1 mM, PPi inhibits calcium phosphate mineral formation by coating hydroxyapatite crystals, thus preventing mineral crystal growth and proliferative self-nucleation. Thus, PPi has a dual physiological role; it can function as a promoter of mineralization at low concentrations but as an inhibitor of mineralization at higher concentrations. TNALP has been shown to hydrolyze the mineralization inhibitor PPi to facilitate mineral precipitation and growth (Rezende et al. 1998). Recent studies using the Akp2^{-/-} mice have indicated that the primary role of TNALP in vivo is to restrict the size of the extracellular PPi pool to allow proper skeletal mineralization (Hessle et al. 2002; Harmey et al. 2004).

The severity of Hypophosphatasia is variable and modulated by the nature of the TNALP mutation. Missense mutations in the enzyme's active site vicinity, homodimer interface, crown domain, amino-terminal arm and calcium-binding site have all been found to affect the catalytic activity of TNALP (Zurutuza et al. 1999). Additionally, other missense, nonsense, frame-shift and splice site mutations have been shown to lead to aberrant mutant proteins or intracellular trafficking defects that lead to subnormal activity on the cell surface. The multitude of mutations and the fact that compound heterozygocity is a common occurrence in Hypophosphatasia also explains the variable expressivity and incomplete penetrance often observed in this disease (Whyte 2001).

Progress on the human form of the disease benefits greatly from the existence of the TNALP null mice (Akp2^{-/-}) as an animal model. These Akp2^{-/-} mice phenocopy infantile HPP remarkably well, as they are born with a normally mineralized skeleton, but develop radiographically apparent rickets at about 6 days of age, and die between day 12-16 suffering severe skeletal hypomineralization and episodes of apnea and epileptic seizures attributable to disturbances in PLP (vitamin B₆) metabolism (Waymire et al. 1995; Narisawa et al. 1997; Fedde et al. 1999; Narisawa et al. 2001).

Some TNALP active site mutations have been shown to affect the ability of the enzyme to metabolize PPi or PLP differently (Di Mauro et al. 2002). Both PLP and PPi are confirmed natural substrates of TNALP and abnormalities in PLP metabolism explain the epileptic seizures observed in Akp2^{-/-} mice (Waymire et al. 1995; Narisawa et al. 2001), while abnormalities in PPi metabolism explain the skeletal phenotype in this mouse model of Hypophosphatasia (Hessle et al. 2002; Anderson et al. 2004; Harmey et al. 2004; Harmey et al. 2006; Anderson et al. 2005a).

There is no established medical therapy for HPP. Case reports of enzyme replacement therapy (ERT) using intravenous (i.v.) infusions of TNALP-rich plasma from Paget bone disease patients and purified placental ALP have described failure to rescue affected infants (Whyte et al. 1982; Whyte et al. 1984). In another similar study, Weninger et al. (Weninger et al. 1989) attempted ERT for a severely affected premature boy with Hypophosphatasia by infusions of purified human liver TNALP. Treatment (1.2 IU/kg/min) started at age three weeks and was repeated in weekly intervals until age 10 weeks, when the child died. Samples of TNALP were diluted with 10 ml of physiological saline and infused over 30 min via an umbilical arterial catheter. No toxic or allergic side effects were observed. Serum TNALP activity increased from 3 IU/L before treatment to a maximum level of 195 IU/L with a half-life time between 37 and 62 hours. Sequential radiographic studies however showed no improvement of bone mineralization (Weninger et al. 1989).

It seems that ALP activity must be increased not in the circulation, but in the skeleton itself. This hypothesis is supported by seemingly beneficial responses of two girls with infantile HPP following marrow cell transplantation where TNALP-containing cells were introduced throughout the skeleton (Whyte et al. 2003). Thus there seems to be a need to provide active TNALP to the skeleton of these patients. Recent reports have indicated that poly-aspartate sequences confer bone homing properties to recombinant TNALP (WO 2005/103263 to Crine et al.; Nishioka et al. 2006).

A recent report showed that the mutated form of TNALP R450C (although Nasu et al. refers to a R433C mutation, his numbering applies to the mature protein and not to one comprising the signal peptide) produces a protein having a dimeric structure joined by a disulfide bridge between the cysteine residues at position 450 of each subunit which strongly inhibited its alkaline phosphatase activity. Nasu et al. concluded that the loss of function results from the interchain disulfide bridge and is the molecular basis for the lethal hypophosphatasia associated with R450C (Nasu et al. 2006).

The present description refers to a number of documents, the content of which is herein incorporated by reference in their entirety.

### SUMMARY OF THE INVENTION

Given the current limitations in the clinical management and treatment of patients with HPP, an alternative and efficient treatment was needed. Accordingly, the present invention provides an efficient enzyme replacement therapy for the treatment of HPP.

To the Applicant's knowledge, and as opposed to previous enzyme replacement therapy efforts in either TNALP null mice or HPP infants in which TNALP or other ALP isozymes were delivered intravenously, the present invention marks the first time where near complete resolution of clinical radiographic and biochemical changes has been documented to occur with enzyme replacement alone.

Bone Targeted sALP

The bone targeted composition of the present invention comprise a fusion protein including in order from the amino side to the carboxylic side a sALP, a spacer, and a bone targeting negatively charged peptide.

### ALPs

There are four known isozymes of ALP, namely tissue non specific alkaline phosphatase further described below, placental alkaline phosphatase (PALP) (e.g., [NP_{—}112603], [NP_{—}001623]), germ cell alkaline phosphatase (GCALP) (e.g., [P10696]) and intestinal alkaline phosphatase (e.g., [NP_{—}001622]). These enzymes possess very similar three dimensional structure. Each of their catalytic sites contains four metal binding domains for metal ions necessary for enzymatic activity including two Zn and one Mg. These enzymes catalyze the hydrolysis of monoesters of phosphoric acid and also catalyze a transphosphorylation reaction in the presence of high concentrations of phosphate acceptors. It has been shown in particular that PALP is physiologically active toward phosphoethanolamine (PEA), inorganic pyrophosphate (PPi) and pyridoxal 5′-phosphate (PLP), all three being known natural substrate for TNALP (Whyte, 1995). An alignment between these isozymes is presented in FIG. 30.

### TNALP

As indicated above, TNALP is a membrane-bound protein anchored through a glycolipid to its C-terminal (Swiss-Prot, P05186). This glycolipid anchor (GPI) is added post translationally after removal of a hydrophobic C-terminal end which serves both as a temporary membrane anchor and as a signal for the addition of the GPI. Hence the soluble human TNALP used in all Examples below is comprised of a TNALP wherein the first amino acid of the hydrophobic C-terminal sequence, namely alanine, is replaced by a stop codon. The soluble TNALP (herein called sTNALP) so formed contains all amino acids of the native anchored form of TNALP necessary for the formation of the catalytic site but lacks the GPI membrane anchor. Known TNALP include human TNALP [NP-000469, AAI10910, AAH90861, AAH66116, AAH21289, AAI26166]; rhesus TNALP [XP-001109717]; rat TNALP [NP_{—}037191]; dog TNALP [AAF64516]; pig TNALP [AAN64273], mouse [NP_{—}031457], bovine [NP_{—}789828, NP_{—}776412, AAI18209, AAC33858], and cat [NP_{—}001036028].

The bone targeted composition of the present invention encompasses sequences satisfying a consensus sequence derived from the ALP extracellular domain of human ALP isozymes and of known functional TNALPs (human, mouse, rat, bovine, cat and dog). As used herein the terminology “extracellular domain” is meant to refer to any functional extracellular portion of the native protein (i.e. without the peptide signal). It has been shown that recombinant sTNALP retaining original amino acids 1 to 501 (18 to 501 when secreted) (see Oda et al., J. Biochem 126: 694-699, 1999), amino acids 1 to 504 (18 to 504 when secreted) (U.S. Pat. No. 6,905,689 to Bernd et al.) and amino acids 1 to 505 (18-505 when secreted) (US 2007/0081984 to Tomatsu et al.), are enzymatically active. Examples presented herein also show that a recombinant sTNALP retaining amino acids 1 to 502 (18 to 502 when secreted) (FIG. 3) of the original TNALP is enzymatically active. This indicates that amino acid residues can be removed from the C-terminal end of the native protein without affecting its enzymatic activity.

Table 1 below provides a list of 194 mutations known to cause HPP. In specific embodiments of the bone targeted polypeptides of present invention, the ALP sequence does not include any of these mutations.

Hence, in sALPs of the present invention, using the numbering of a consensus sequence derived from an alignment of various TNALPs and of human ALP isozymes, the amino acid at position 22 is not a phenylalanine residue; the amino acid at position 33 (position 11 in the sequence without signal peptide) is not a cysteine residue; the amino acid at position 38 (position 16 in the sequence without signal peptide) is not a valine residue; the amino acid at position 42 (position 20 in the sequence without signal peptide) is not a proline residue; the amino acid at position 45 (position 23 in the sequence without signal peptide) is not a valine residue; the amino acid residue at position 56 (position 34 in the sequence without signal peptide) is not a serine or a valine residue; the amino acid residue at position 67 (position 45 in the sequence without signal peptide) is not a leucine, an isoleucine or a valine residue; the amino acid residue at position 68 (position 46 in the sequence without signal peptide) is not a valine residue; the amino acid residue at position 73 (position 51 in the sequence without signal peptide) is not a methionine residue; the amino acid residue at position 76 (position 54 in the sequence without signal peptide) is not a cysteine, a serine, a proline or a histidine residue; the amino acid residue at position 77 (position 55 in the sequence without signal peptide) is not a threonine residue; the amino acid residue at position 80 (position 58 in the sequence without signal peptide) is not a serine residue; the amino acid residue at position 81 (position 59 in the sequence without signal peptide) is not an asparagine residue; the amino acid residue at position 105 (position 83 in the sequence without signal peptide) is not a methionine residue; the amino acid residue at position 113 (position 89 in the sequence without signal peptide) is not a leucine residue; the amino acid residue at position 116 (position 94 in the sequence without signal peptide) is not a threonine residue; the amino acid residue at position 117 (position 95 in the sequence without signal peptide) is not a serine residue; the amino acid residue at position 119 (position 97 in the sequence without signal peptide) is not a glycine residue; the amino acid residue at position 121 (position 99 in the sequence without signal peptide) is not a serine or a threonine residue; the amino acid residue at position 125 (position 103 in the sequence without signal peptide) is not an arginine residue; the amino acid residue at position 128 (position 106 in the sequence without signal peptide) is not a aspartate residue; the amino acid residue at position 133 (position 111 in the sequence without signal peptide) is not a methionine residue; the amino acid residue at position 134 (position 112 in the sequence without signal peptide) is not an arginine residue; the amino acid residue at position 137 (position 115 in the sequence without signal peptide) is not a threonine or a valine residue; the amino acid residue at position 139 (position 117 in the sequence without signal peptide) is not a histidine or an asparagine residue; the amino acid residue at position 141 (position 119 in the sequence without signal peptide) is not a histidine residue; the amino acid residue at position 153 (position 131 in the sequence without signal peptide) is not an alanine or an isoleucine residue; the amino acid residue at position 167 (position 145 in the sequence without signal peptide) is not a serine or a valine residue; the amino acid residue at position 172 (position 150 in the sequence without signal peptide) is not a methionine residue; the amino acid residue at position 175 (position 153 in the sequence without signal peptide) is not an aspartate residue; the amino acid residue at position 176 (position 154 in the sequence without signal peptide) is not a tyrosine or an arginine residue; the amino acid residue at position 181 (position 159 in the sequence without signal peptide) is not a threonine residue; the amino acid residue at position 182 (position 160 in the sequence without signal peptide) is not a threonine residue; the amino acid residue at position 184 (position 162 in the sequence without signal peptide) is not a threonine residue; the amino acid residue at position 186 (position 164 in the sequence without signal peptide) is not a leucine residue; the amino acid residue at position 189 (position 167 in the sequence without signal peptide) is not a tryptophan residue; the amino acid residue at position 194 (position 172 in the sequence without signal peptide) is not a glutamate residue; the amino acid residue at position 196 (position 174 in the sequence without signal peptide) is not a lysine or a glycine residue; the amino acid residue at position 197 (position 175 in the sequence without signal peptide) is not a threonine residue; the amino acid residue at position 198 (position 176 in the sequence without signal peptide) is not an alanine residue; the amino acid residue at position 206 (position 184 in the sequence without signal peptide) is not a tyrosine residue; the amino acid residue at position 208 (position 186 in the sequence without signal peptide) is not a glutamate residue; the amino acid residue at position 207 (position 190 in the sequence without signal peptide) is not a proline residue; the amino acid residue at position 216 (position 194 in the sequence without signal peptide) is not a aspartate residue; the amino acid residue at position 217 (position 195 in the sequence without signal peptide) is not a phenylalanine residue; the amino acid residue at position 223 (position 201 in the sequence without signal peptide) is not a threonine residue; the amino acid residue at position 225 (position 203 in the sequence without signal peptide) is not a valine or an alanine residue; the amino acid residue at position 226 (position 204 in the sequence without signal peptide) is not a valine residue; the amino acid residue at position 228 (position 206 in the sequence without signal peptide) is not a tryptophan or a glutamine residue; the amino acid residue at position 229 (position 207 in the sequence without signal peptide) is not a glutamate residue; the amino acid residue at position 231 (position 209 in the sequence without signal peptide) is not a threonine residue; the amino acid residue at position 240 (position 218 in the sequence without signal peptide) is not a glycine residue; the amino acid residue at position 251 (position 229 in the sequence without signal peptide) is not a serine residue; the amino acid residue at position 254 (position 232 in the sequence without signal peptide) is not a valine residue; the amino acid residue at position 269 (position 247 in the sequence without signal peptide) is not an arginine residue; the amino acid residue at position 277 (position 255 in the sequence without signal peptide) is not a cysteine, a leucine or a histidine residue; the amino acid residue at position 280 (position 258 in the sequence without signal peptide) is not a proline residue; the amino acid residue at position 295 (position 273 in the sequence without signal peptide) is not a phenylalanine residue; the amino acid residue at position 297 (position 275 in the sequence without signal peptide) is not a lysine residue; the amino acid residue at position 298 (position 276 in the sequence without signal peptide) is not a threonine residue; the amino acid residue at position 300 (position 278 in the sequence without signal peptide) is not a tyrosine or an alanine residue; the amino acid residue at position 301 (position 279 in the sequence without signal peptide) is not a valine, a threonine or an isoleucine residue; the amino acid residue at position 303 (position 281 in the sequence without signal peptide) is not an aspirate residue; the amino acid residue at position 304 (position 282 in the sequence without signal peptide) is not a lysine residue; the amino acid residue at position 305 (position 283 in the sequence without signal peptide) is not a proline residue; the amino acid residue at position 312 (position 290 in the sequence without signal peptide) is not a valine residue; the amino acid residue at position 313 (position 291 in the sequence without signal peptide) is not a serine or a leucine residue; the amino acid residue at position 317 (position 295 in the sequence without signal peptide) is not a lysine residue; the amino acid residue at position 332 (position 310 in the sequence without signal peptide) is not an arginine residue; the amino acid residue at position 333 (position 311 in the sequence without signal peptide) is not a cysteine, a glycine or a leucine residue; the amino acid residue at position 334 (position 312 in the sequence without signal peptide) is not a leucine residue; the amino acid residue at position 340 (position 318 in the sequence without signal peptide) is not an aspartate residue; the amino acid residue at position 345 (position 323 in the sequence without signal peptide) is not an arginine or a glutamate residue; the amino acid residue at position 354 (position 332 in the sequence without signal peptide) is not a threonine residue; the amino acid residue at position 360 (position 338 in the sequence without signal peptide) is not an aspartate residue; the amino acid residue at position 361 (position 339 in the sequence without signal peptide) is not a threonine or an isoleucine residue; the amino acid residue at position 377 (position 355 in the sequence without signal peptide) is not a leucine residue; the amino acid residue at position 380 (position 358 in the sequence without signal peptide) is not a methionine residue; the amino acid residue at position 383 (position 361 in the sequence without signal peptide) is not a valine residue; the amino acid residue at position 384 (position 362 in the sequence without signal peptide) is not a valine residue; the amino acid residue at position 387 (position 365 in the sequence without signal peptide) is not an arginine residue; the amino acid residue at position 388 (position 366 in the sequence without signal peptide) is not a leucine residue; the amino acid residue at position 395 (position 373 in the sequence without signal peptide) is not a leucine residue; the amino acid residue at position 397 (position 375 in the sequence without signal peptide) is not a cysteine or a histidine residue; the amino acid residue at position 398 (position 376 in the sequence without signal peptide) is not an alanine residue; the amino acid residue at position 401 (position 379 in the sequence without signal peptide) is not a threonine residue; the amino acid residue at position 405 (position 383 in the sequence without signal peptide) is not a serine or a valine residue; the amino acid residue at position 406 (position 384 in the sequence without signal peptide) is not a leucine residue; the amino acid residue at position 412 (position 390 in the sequence without signal peptide) is not a glycine residue; the amino acid residue at position 416 (position 394 in the sequence without signal peptide) is not a leucine residue; the amino acid residue at position 417 (position 395 in the sequence without signal peptide) is not an alanine residue; the amino acid residue at position 420 (position 398 in the sequence without signal peptide) is not a methionine residue; the amino acid residue at position 423 (position 401 in the sequence without signal peptide) is not a serine residue; the amino acid residue at position 426 (position 404 in the sequence without signal peptide) is not a serine residue; the amino acid residue at position 429 (position 407 in the sequence without signal peptide) is not an alanine residue; the amino acid residue at position 430 (position 408 in the sequence without signal peptide) is not a methionine residue; the amino acid residue at position 432 (position 410 in the sequence without signal peptide) is not a cysteine or an aspartate residue; amino acid residue at position 434 (position 412 in the sequence without signal peptide) is not a proline residue; amino acid residue at position 435 (position 413 in the sequence without signal peptide) is not a lysine residue; amino acid residue at position 442 (position 420 in the sequence without signal peptide) is not a histidine residue; amino acid residue at position 451 (position 429 in the sequence without signal peptide) is not a proline residue; amino acid residue at position 456 (position 434 in the sequence without signal peptide) is not a histidine or a cysteine residue; amino acid residue at position 458 (position 436 in the sequence without signal peptide) is not a lysine residue; amino acid residue at position 460 (position 438 in the sequence without signal peptide) is not an arginine residue; amino acid residue at position 461 (position 439 in the sequence without signal peptide) is not a serine or an aspartate residue; amino acid residue at position 462 (position 440 in the sequence without signal peptide) is not a tryptophan or an arginine residue; amino acid residue at position 465 (position 443 in the sequence without signal peptide) is not a methionine or a leucine residue; amino acid residue at position 472 (position 450 in the sequence without signal peptide) is not a leucine residue; amino acid residue at position 473 (position 451 in the sequence without signal peptide) is not a threonine residue; amino acid residue at position 474 (position 452 in the sequence without signal peptide) is not a threonine residue; amino acid residue at position 479 (position 457 in the sequence without signal peptide) is not a serine residue; amino acid residue at position 482 (position 460 in the sequence without signal peptide) is not a lysine or a glycine residue; amino acid residue at position 484 (position 462 in the sequence without signal peptide) is not a leucine residue; amino acid residue at position 495 (position 473 in the sequence without signal peptide) is not a serine residue; amino acid residue at position 496 (position 474 in the sequence without signal peptide) is not a phenylalanine residue; and amino acid residue at position 497 (position 475 in the sequence without signal peptide) is not an arginine residue.

Also more specifically, when a sTNALP is used in the bone targeted sALPs of the present invention, using the numbering of the human TNALP sequence, the amino acid at position 17 is not a phenylalanine residue; the amino acid at position 28 (position 11 in the sequence without signal peptide) is not a cysteine residue; the amino acid at position 33 (position 16 in the sequence without signal peptide) is not a valine residue; the amino acid at position 37 (position 20 in the sequence without signal peptide) is not a proline residue; the amino acid at position 40 (position 23 in the sequence without signal peptide) is not a valine residue; the amino acid residue at position 51 (position 34 in the sequence without signal peptide) is not a serine or a valine residue; the amino acid residue at position 62 (position 45 in the sequence without signal peptide) is not a leucine, an isoleucine or a valine residue; the amino acid residue at position 63 (position 46 in the sequence without signal peptide) is not a valine residue; the amino acid residue at position 68 (position 51 in the sequence without signal peptide) is not a methionine residue; the amino acid residue at position 71 (position 54 in the sequence without signal peptide) is not a cysteine, a serine, a proline or a histidine residue; the amino acid residue at position 72 (position 55 in the sequence without signal peptide) is not a threonine residue; the amino acid residue at position 75 (position 58 in the sequence without signal peptide) is not a serine residue; the amino acid residue at position 76 (position 59 in the sequence without signal peptide) is not an asparagine residue; the amino acid residue at position 100 (position 83 in the sequence without signal peptide) is not a methionine residue; the amino acid residue at position 108 (position 89 in the sequence without signal peptide) is not a leucine residue; the amino acid residue at position 111 (position 94 in the sequence without signal peptide) is not a threonine residue; the amino acid residue at position 112 (position 95 in the sequence without signal peptide) is not a serine residue; the amino acid residue at position 114 (position 97 in the sequence without signal peptide) is not a glycine residue; the amino acid residue at position 116 (position 99 in the sequence without signal peptide) is not a serine or a threonine residue; the amino acid residue at position 120 (position 103 in the sequence without signal peptide) is not an arginine residue; the amino acid residue at position 123 (position 106 in the sequence without signal peptide) is not a aspartate residue; the amino acid residue at position 128 (position 111 in the sequence without signal peptide) is not a methionine residue; the amino acid residue at position 129 (position 112 in the sequence without signal peptide) is not an arginine residue; the amino acid residue at position 132 (position 115 in the sequence without signal peptide) is not a threonine or a valine residue; the amino acid residue at position 134 (position 117 in the sequence without signal peptide) is not a histidine or an asparagine residue; the amino acid residue at position 136 (position 119 in the sequence without signal peptide) is not a histidine residue; the amino acid residue at position 148 (position 131 in the sequence without signal peptide) is not an alanine or an isoleucine residue; the amino acid residue at position 162 (position 145 in the sequence without signal peptide) is not a serine or a valine residue; the amino acid residue at position 167 (position 150 in the sequence without signal peptide) is not a methionine residue; the amino acid residue at position 170 (position 153 in the sequence without signal peptide) is not an aspartate residue; the amino acid residue at position 171 (position 154 in the sequence without signal peptide) is not a tyrosine or an arginine residue; the amino acid residue at position 176 (position 159 in the sequence without signal peptide) is not a threonine residue; the amino acid residue at position 177 (position 160 in the sequence without signal peptide) is not a threonine residue; the amino acid residue at position 179 (position 162 in the sequence without signal peptide) is not a threonine residue; the amino acid residue at position 181 (position 164 in the sequence without signal peptide) is not a leucine residue; the amino acid residue at position 184 (position 167 in the sequence without signal peptide) is not a tryptophane residue; the amino acid residue at position 189 (position 172 in the sequence without signal peptide) is not a glutamate residue; the amino acid residue at position 191 (position 174 in the sequence without signal peptide) is not a lysine or a glycine residue; the amino acid residue at position 192 (position 175 in the sequence without signal peptide) is not a threonine residue; the amino acid residue at position 193 (position 176 in the sequence without signal peptide) is not an alanine residue; the amino acid residue at position 201 (position 184 in the sequence without signal peptide) is not a tyrosine residue; the amino acid residue at position 203 (position 186 in the sequence without signal peptide) is not a glutamate residue; the amino acid residue at position 207 (position 190 in the sequence without signal peptide) is not a proline residue; the amino acid residue at position 211 (position 194 in the sequence without signal peptide) is not a aspartate residue; the amino acid residue at position 212 (position 195 in the sequence without signal peptide) is not a phenylalanine residue; the amino acid residue at position 218 (position 201 in the sequence without signal peptide) is not a threonine residue; the amino acid residue at position 220 (position 203 in the sequence without signal peptide) is not a valine or an alanine residue; the amino acid residue at position 221 (position 204 in the sequence without signal peptide) is not a valine residue; the amino acid residue at position 223 (position 206 in the sequence without signal peptide) is not a tryptophane or a glutamine residue; the amino acid residue at position 224 (position 207 in the sequence without signal peptide) is not a glutamate residue; the amino acid residue at position 226 (position 209 in the sequence without signal peptide) is not a threonine residue; the amino acid residue at position 235 (position 218 in the sequence without signal peptide) is not a glycine residue; the amino acid residue at position 246 (position 229 in the sequence without signal peptide) is not a serine residue; the amino acid residue at position 249(position 232 in the sequence without signal peptide) is not a valine residue; the amino acid residue at position 264 (position 247 in the sequence without signal peptide) is not an arginine residue; the amino acid residue at position 272 (position 255 in the sequence without signal peptide) is not a cysteine, a leucine or a histidine residue; the amino acid residue at position 275 (position 258 in the sequence without signal peptide) is not a proline residue; the amino acid residue at position 289 (position 272 in the sequence without signal peptide) is not a phenylalanine residue; the amino acid residue at position 291(position 274 in the sequence without signal peptide) is not a lysine residue; the amino acid residue at position 292 (position 275 in the sequence without signal peptide) is not a threonine residue; the amino acid residue at position 294 (position 277 in the sequence without signal peptide) is not a tyrosine or an alanine residue; the amino acid residue at position 295 (position 278 in the sequence without signal peptide) is not a valine, a threonine or an isoleucine residue; the amino acid residue at position 297 (position 280 in the sequence without signal peptide) is not an aspirate residue; the amino acid residue at position 298 (position 281 in the sequence without signal peptide) is not a lysine residue; the amino acid residue at position 299 (position 282 in the sequence without signal peptide) is not a proline residue; the amino acid residue at position 306 (position 289 in the sequence without signal peptide) is not a valine residue; the amino acid residue at position 307 (position 290 in the sequence without signal peptide) is not a serine or a leucine residue; the amino acid residue at position 311 (position 294 in the sequence without signal peptide) is not a lysine residue; the amino acid residue at position 326 (position 309 in the sequence without signal peptide) is not an arginine residue; the amino acid residue at position 327 (position 310 in the sequence without signal peptide) is not a cysteine, a glycine or a leucine residue; the amino acid residue at position 328 (position 311 in the sequence without signal peptide) is not a leucine residue; the amino acid residue at position 334 (position 317 in the sequence without signal peptide) is not an aspartate residue; the amino acid residue at position 339 (position 322 in the sequence without signal peptide) is not an arginine or a glutamate residue; the amino acid residue at position 348 (position 331 in the sequence without signal peptide) is not a threonine residue; the amino acid residue at position 354 (position 337 in the sequence without signal peptide) is not an aspartate residue; the amino acid residue at position 355 (position 338 in the sequence without signal peptide) is not a threonine or an isoleucine residue; the amino acid residue at position 371 (position 354 in the sequence without signal peptide) is not a leucine residue; the amino acid residue at position 374 (position 357 in the sequence without signal peptide) is not a methionine residue; the amino acid residue at position 377 (position 360 in the sequence without signal peptide) is not a valine residue; the amino acid residue at position 378 (position 361 in the sequence without signal peptide) is not a valine residue; the amino acid residue at position 381 (position 364 in the sequence without signal peptide) is not an arginine residue; the amino acid residue at position 382 (position 365 in the sequence without signal peptide) is not a leucine residue; the amino acid residue at position 389 (position 372 in the sequence without signal peptide) is not a leucine residue; the amino acid residue at position 391 (position 374 in the sequence without signal peptide) is not a cysteine or a histidine residue; the amino acid residue at position 392 (position 375 in the sequence without signal peptide) is not an alanine residue; the amino acid residue at position 395 (position 378 in the sequence without signal peptide) is not a threonine residue; the amino acid residue at position 399 (position 382 in the sequence without signal peptide) is not a serine or a valine residue; the amino acid residue at position 400 (position 383 in the sequence without signal peptide) is not a leucine residue; the amino acid residue at position 406 (position 389 in the sequence without signal peptide) is not a glycine residue; the amino acid residue at position 410 (position 393 in the sequence without signal peptide) is not a leucine residue; the amino acid residue at position 411 (position 394 in the sequence without signal peptide) is not an alanine residue; the amino acid residue at position 414 (position 397 in the sequence without signal peptide) is not a methionine residue; the amino acid residue at position 417 (position 400 in the sequence without signal peptide) is not a serine residue; the amino acid residue at position 420 (position 403 in the sequence without signal peptide) is not a serine residue; the amino acid residue at position 423 (position 406 in the sequence without signal peptide) is not an alanine residue; the amino acid residue at position 424 (position 407 in the sequence without signal peptide) is not a methionine residue; the amino acid residue at position 426 (position 409 in the sequence without signal peptide) is not a cysteine or an aspartate residue; amino acid residue at position 428 (position 411 in the sequence without signal peptide) is not a proline residue; amino acid residue at position 429 (position 412 in the sequence without signal peptide) is not a lysine residue; amino acid residue at position 436 (position 419 in the sequence without signal peptide) is not a histidine residue; amino acid residue at position 445 (position 428 in the sequence without signal peptide) is not a proline residue; amino acid residue at position 450 (position 433 in the sequence without signal peptide) is not a histidine or a cysteine residue; amino acid residue at position 452 (position 435 in the sequence without signal peptide) is not a lysine residue; amino acid residue at position 454 (position 437 in the sequence without signal peptide) is not an arginine residue; amino acid residue at position 455 (position 438 in the sequence without signal peptide) is not a serine or an aspartate residue; amino acid residue at position 456 (position 439 in the sequence without signal peptide) is not a tryptophane or an arginine residue; amino acid residue at position 459 (position 442 in the sequence without signal peptide) is not a methionine or a leucine residue; amino acid residue at position 466 (position 449 in the sequence without signal peptide) is not a leucine residue; amino acid residue at position 467 (position 450 in the sequence without signal peptide) is not a threonine residue; amino acid residue at position 468 (position 451 in the sequence without signal peptide) is not a threonine residue; amino acid residue at position 473 (position 456 in the sequence without signal peptide) is not a serine residue; amino acid residue at position 476 (position 459 in the sequence without signal peptide) is not a lysine or a glycine residue; amino acid residue at position 478 (position 461 in the sequence without signal peptide) is not a leucine residue; amino acid residue at position 489 (position 472 in the sequence without signal peptide) is not a serine residue; amino acid residue at position 490 (position 473 in the sequence without signal peptide) is not a phenylalanine residue; and amino acid residue at position 491 (position 474 in the sequence without signal peptide) is not an arginine residue. In other specific embodiments, one or more Xs are defined as being any of the amino acids found at that position in the sequences of the alignment or a residue that constitutes a conserved or semi-conserved substitution of any of these amino acids. In other specific embodiments, Xs are defined as being any of the amino acids found at that position in the sequences of the alignment. For instance, the amino acid residue at position 51 (position 34 in the sequence without signal peptide) is an alanine or a valine residue; the amino acid residue at position 177 (position 160 in the sequence without signal peptide) is an alanine or a serine residue; the amino acid residue at position 212 (position 195 in the sequence without signal peptide) is an isoleucine or a valine residue; the amino acid residue at position 291 (position 274 in the sequence without signal peptide) is a glutamic acid or an aspartic acid residue; and the amino acid residue at position 374 (position 357 in the sequence without signal peptide) is a valine or an isoleucine residue.

In specific embodiments, the sALP fragment in the bone targeted fusion protein of the present invention consists of any one of the fragments of a consensus sequence derived from an alignment of human ALP isozymes and TNALPs from various mammalian species corresponding to amino acid residues 18-498, 18-499, 18-500, 18-501, 18-502, 18-503, 18-504, or 18 to 505 of human TNALP. These consensus fragments are amino acid residues 23 to 508, 23 to 509, 23 to 510, 23 to 511, 23 to 512, 23 to 513, 23 to 514 and 23 to 515 of SEQ ID NO: 15, respectively. In these consensus fragments, X is any amino acid except an amino acid corresponding to a pathological mutation at that position of human TNALP as reported in Table 1. In other specific embodiments, these consensus fragments are amino acid residues 23 to 508, 23 to 509, 23 to 510, 23 to 511, 23 to 512, 23 to 513, 23 to 514 and 23 to 515 of SEQ ID NO: 18, respectively. In these consensus fragments, X is any amino acid found at that position in the ALP of either one of the species and human ALP isozymes of the alignment from which the consensus is derived but is not an amino acid corresponding to a pathological mutation at that position of human TNALP as reported in Table 1 (See FIG. 30).

In other specific embodiments, the sALP fragment in the bone targeted fusion protein of the present invention consist of any of the fragments of a consensus sequence derived from an alignment of TNALPs from various mammalian species corresponding to amino acid residues 18-498, 18-499, 18-500, 18-501, 18-502, 18-503, 18-504, and 18 to 505 of human TNALP. These consensus fragments are amino acid residues 18-498, 18-499, 18-500, 18-501, 18-502, 18-503, 18-504, and 18 to 505 of SEQ ID NO: 16, respectively. In these consensus fragments, X is any amino acid except an amino acid corresponding to a pathological mutation at that position of human TNALP as reported in Table 1. In other specific embodiments, these consensus fragments are amino acid residues 18-498, 18-499, 18-500, 18-501, 18-502, 18-503, 18-504, and 18 to 505 of SEQ ID NO: 19, respectively. In these consensus fragments, X is any amino acid found at that position in the TNALP of either one of the species of the alignment from which the consensus is derived but is not an amino acid corresponding to a pathological mutation at that position of human TNALP as reported in Table 1 (See FIG. 31).

TABLE 1
Pathological mutations in human TNALP
Total number of mutations 188

| | | Amino acid change | | | | | | | | |
|---|---|---|---|---|---|---|---|---|---|---|
| | | Non- | | | Clinical | | | | | |
| | | standardized | Standardized | | form in | Genotype of | | | | |
| Exon | Base change | nomenclature | nomenclature | Reference | patient | patient | % WT | ref. | E. coli | |
| 1 | c.-195C > T | | | Taillandier et al. 2000 | perinatal | c.- | | | na | Affects transcription |
| | | | | | | 195C > T/C184Y | | | | start site |
| 2 | c.17T > A | L-12X | p.L6X | Taillandier et al. 2000 | childhood | L-12X/? | | | na | Nonsense mutation |
| 2 | c.50C > T | S-1F | p.S17F | Mornet et al. 1998 | infantile | S-1F/G58S | 19.0 | 1 | na | |
| 3 | c.83A > G | Y11C | p.Y28C | Taillandier et al. 2001 | infantile | Y11C/R119H | 7.2 | 2 | − | |
| 3 | c.98C > T | A16V | p.A33V | Henthorn et al. 1992 | childhood | A16V/Y419H | | | − | |
| 3 | c.110T > C | L20P | p.L37P | Versailles lab october 2003 | perinatal | L20P/L20P | | | + | |
| 3 | c.119C > T | A23V | p.A40V | Mornet et al. 1998 | perinatal | A23V/G456S | 2.3 | 1 | + | |
| 3 | c.132C > T | Q27X | p.Q44X | Mornet E, unpublished | perinatal | Q27X/c.662insG | | | na | Nonsense mutation |
| 3 | c.151G > T | A34S | p.A51S | Mumm et al. 2002 | infantile | A34S/T117H | | | + | |
| 3 | c.152G > T | A34V | p.A51V | Taillandier et al. 2001 | infantile | A34V/V442M | | | + | |
| 4 | c.184A > T | M45L | p.M62L | Taillandier et al. 1999 | infantile | M45L/c.1172delC | 27.4 | 1 | + | |
| 4 | c.184A > G | M45V | p.M62V | Spentchian et al. 2003 | infantile | M45V/M45V | | | + | |
| 4 | c.186G > C | M45I | p.M62I | Taillandier et al. 2005 | childhood | M45I/E174K | 0 | 16 | + | |
| 4 | c.187G > C | G46R | p.G63R | Spentchian et al. 2003 | infantile | G46R/G46R | | | + | |
| 4 | c.188G > T | G46V | p.G63V | Lia-Baldini et al. 2001 | infantile | G46V/N | 0.8 | 3 | + | |
| 4 | c.203C > T | T51M | p.T68M | Orimo et al. 2002 | childhood | T51M/A160T | 5.2 | 4 | + | |
| 4 | c.211C > T | R54C | p.R71C | Henthorn et al. 1992 | infantile | R54C/D277A | 0 | 17 | + | |
| 4 | c.211C > A | R54S | p.R71S | Orimo et al. 2002 | childhood | R54S/? | 2.9 | 4 | + | |
| 4 | c.212G > C | R54P | p.R71P | Henthorn et al. 1992 | perinatal | R54P/Q190P | | | + | |
| 4 | c.212G > A | R54H | p.R71H | Taillandier et al. 2001 | perinatal | A23V/R54H | | | + | |
| 4 | c.219T > C | I55T | p.I72T | Versailles lab october 2004 | odonto | I55T/N | | | − | |
| 4 | c.223G > A | G58S | p.G75S | Mornet et al. 1998 | infantile | S-1F/G58S | 3.5 | 1 | + | |
| 4 | c.227A > G | Q59R | p.Q76R | Mornet et al. 2001 | infantile | Q59R/T117N | | | − | |
| IVS4 | c.298-2A > G | | | Taillandier et al. 2000 | perinatal | c.298- | | | na | This mutation affects |
| | | | | | | 2A > G/c.997 + 3A > C | | | | splicing and not coding |
| | | | | | | | | | | sequence |
| 5 | c.299C > T | T83M | p.T100M | Mornet et al. 2001 | infantile | T83M/E174K | | | + | |
| 5 | c.303_311del | N85_N87del | p.N102_N104del | Versailles lab July 2007 | perinatal | c.303_311del/G474R | | | na | Deletion |
| 5 | c.323C > T | P91L | p.P108L | Herasse et al. 2003 | odonto | P91L/N | 0.4 | unp. | − | |
| 5 | c.331G > A | A94T | p.A111T | Goseki-Sone et al. | odonto | A94T/? | | | + | |
| | | | | 1998 | | | | | | |
| 5 | c.334G > A | G95S | p.G112S | Witters et al. 2004 | infantile | G95S/R374C | | | − | |
| 5 | c.340G > A | A97T | p.A114T | Mumm et al. 2001 | infantile | A97T/D277A | | | + | |
| 5 | c.341C > G | A97G | p.A114G | Draguet et al. 2004 | perinatal | A97G + c.348_349insACCGTC/ | | | + | |
| | | | | | | G309R | | | | |
| 5 | c.348_349insACCGTC | | | Draguet et al. 2004 | perinatal | A97G + c.348_349insACCGTC/ | | | na | Two missense |
| | | | | | | G309R | | | | mutations and insertion |
| 5 | c.346G > T | A99S | p.A116S | Versailles lab July 2007 | adult | A99S/N400S | | | + | |
| 5 | c.346G > A | A99T | p.A116T | Hu et al. 2000 | adult | A99T/N | 0.8 | 3 | + | |
| 5 | c.358G > A | G103R | p.G120R | Mornet et al. 1998 | perinatal | G103R/648 + 1G > A | | | + | |
| 5 | c.368C > A | A106D | p.A123D | Spentchian et al. 2006 | perinatal | A106D/S249_H250del | | | − | |
| 5 | c.382G > A | V111M | p.V128M | Mumm et al. 2002 | perinatal | V111M/R206W | | | − | |
| 5 | c.385G > A | G112R | p.G129R | Mornet et al. 1998 | perinatal | G112R/G474R | | | + | |
| 5 | c.388_391delGTAA | | | Spentchian et al. 2003 | perinatal | E294K/388_391delGTAA | | | na | Frameshift mutation |
| 5 | c.389delT | | | Spentchian et al. 2003 | perinatal | c.389delT/c.389delT | | | na | Frameshift mutation |
| 5 | c.392delG | | | Mumm et al. 2002 | perinat/infant | c.392delG/A331T | | | na | Frameshift mutation |
| 5 | c.394G > A | A115T | p.A132T | Versailles lab July 2006 | adult | A115T/E174K | | | | |
| 5 | c.395C > T | A115V | p.A132V | Watanabe et al. 2001 | adult | A115V/? | 16.9 | 14 | − | |
| 5 | c.400_401AC > | T117H | p.T134H | Mumm et al. 2002 | perinatal | T117H/F310del | | | − | |
| | CA | | | | | | | | | |
| 5 | c.401C > A | T117N | p.T134N | Taillandier et al. 2000 | perinatal | T117N/T117N | 20.5 | 5 | − | |
| 5 | c.406C > T | R119C | p.R136C | Versailles lab october 2003 | odonto | R119C/R119H | | | − | |
| 5 | c.407G > A | R119H | p.R136H | Taillandier et al. 1999 | infantile | R119H/G145V | 33.4 | 1 | − | |
| 5 | c.442A > G | T131A | p.T148A | Michigami et al. 2005 | perinatal | T131A/? | | | − | |
| 5 | c.443C > T | T131I | p.T148I | Spentchian et al. 2003 | infantile | T131I/G145S | | | − | |
| 6 | c.480delT | | | Versailles lab. January | perinatal | c.480delT/R206W | | | na | deletion |
| | | | | 2008 | | | | | | |
| 6 | c.484G > A | G145S | p.G162S | Spentchian et al. 2003 | infantile | T131I/G145S | | | + | |
| 6 | c.485G > T | G145V | p.G162V | Taillandier et al. 1999 | infantile | R119H/G145V | 1.3 | 1 | + | |
| 6 | c.500C > T | T150M | p.T167M | Versailles lab october 2003 | infantile | T150M/E174K | 0 | | + | |
| 6 | c.508A > G | N153D | p.N170D | Mornet et al. 1998 | perinatal | N153D/N153D | 0 | 13 | − | |
| 6 | c.511C > T | H154Y | p.H171Y | Taillandier et al. 1999 | infantile | H154Y/E174K | 2.1 | 1 | − | |
| 6 | c.512A > G | H154R | p.H171R | Mornet E, unpublished | adult | H154R/E174K | | | − | |
| 6 | c.526G > A | A159T | p.A176T | Taillandier et al. 2000 | childhood | A159T/R229S | 45.4 | 5 | + | |
| 6 | c.529G > A | A160T | p.A177T | Goseki-Sone et al. | adult | A160T/F310L | 83.8 | 4 | − | |
| | | | | 1998 | | | | | | |
| 6 | c.535G > A | A162T | p.A179T | Weiss et al. 1988 | perinatal | A162T/A162T | 18 | 6 | + | |
| 6 | c.542C > T | S164L | p.S181L | Lia-Baldini et al. 2001 | infantile | S164L/del(ex12) | 1.3 | 3 | − | |
| 6 | c.544delG | | | Taillandier et al. 1999 | perinatal | G232V/544delG | | | na | Frameshift mutation |
| 6 | c.550C > T | R167W | p.R184W | Mornet et al. 1998 | perinatal | R167W/W253X | 0.6 | 3 | + | |
| 6 | c.567C > A | D172E | p.D189E | Spentchian et al. 2003 | perinatal | D172E/D172E | | | − | |
| 6 | c.568_570delAAC | N173del | p.N190del | Michigami et al. 2005 | perinatal | c.1559delT/N173del | | | − | Deletion of 1 a.a. |
| 6 | c.571G > A | E174K | p.E191K | Henthorn et al. 1992 | infantile | E174K/D361V | 88.0 | 1 | − | |
| 6 | c.572A > G | E174G | p.E191G | Goseki-Sone et al. | odonto | E174G/c.1559delT | | | − | |
| | | | | 1998 | | | | | | |
| 6 | c.575T > C | M175T | p.M192T | Versailles lab July 2007 | infantile | M175T/E294K | | | − | |
| 6 | c.577C > G | P176A | p.P193A | Mumm et al. 2002 | adult | A97T/P176A | | | + | |
| 6 | c.602G > A | C184Y | p.C201Y | Taillandier et al. 1999 | perinatal | c.- | | | − | |
| | | | | | | 195C > T/C184Y | | | | |
| 6 | c.609C > G | D186E | p.D203E | Versailles lab october 2004 | perinatal | D186E/D186E | | | − | |
| 6 | c.620A > C | Q190P | p.Q207P | Henthorn et al. 1992 | perinatal | R54P/Q190P | | | + | |
| 6 | c.631A > G | N194D | p.N211D | Taillandier et al. 2001 | infantile | A99T/N194D | | | + | |
| 6 | c.634A > T | I195F | p.I212F | Souka et al. 2002 | perinatal | I195F/E337D | | | − | |
| IVS6 | c.648 + 1G > T | | | Brun-Heath et al. 2005 | perinatal | c.648 + 1G > T/ | | | | Affects splicing |
| | | | | | | D277A | | | | |
| IVS6 | c.648 + 1G > A | | | Mornet et al. 1998 | perinatal | G103R/c.648 + | | | na | Affects splicing |
| | | | | | | 1G > A | | | | |
| IVS6 | c.649- | | | Versailles lab July 2006 | perinatal | c.649- | | | | Frameshift mutation |
| | 1_3delinsAA | | | | | 1_3delinsAA/c. | | | | |
| | | | | | | 649- | | | | |
| | | | | | | 1_3delinsAA | | | | |
| 7 | c.653T > C | I201T | p.I218T | Utsch et al., 2005, | perinatal | I201T/R374C | 3.7 | unp. | − | |
| | | | | contact | | | | | | |
| 7 | 659G > T | G203V | p.G220V | Taillandier et al. 2001 | odonto | E174K/G203V | | | + | |
| 7 | 659G > C | G203A | p.G220A | Spentchian et al. 2003 | perinatal | G203A/G203A | | | + | |
| 7 | 662insG | | | Mornet E, unpublished | perinatal | Q27X/662insG | | | na | Frameshift mutation |
| 7 | c.662delG | | | Spentchian et al. 2003 | perinatal | R255L/c.662delG | | | na | Frameshift mutation |
| 7 | c.662G > T | G204V | p.G221V | Versailles lab october 2004 | perinatal | G204V/M338T | | | + | |
| 7 | c.667C > T | R206W | p.R223W | Mornet et al. 1998 | perinatal | R206W/? | 2.8 | 3 | − | |
| 7 | c.668G > A | R206Q | p.R223Q | Mumm et al. 2002 | perinatal | R206Q/deletion | | | − | |
| 7 | c.670A > G | K207E | p.K224E | Mochizuki et al. 2000 | infantile | K207E/G409C | 43 | 15 | + | |
| 7 | c.677T > C | M209T | p.M226T | Baumgartner-Sigl et al. | infantile | M209T/T354I | | | − | |
| | | | | 2007 | | | | | | |
| 7 | c.704A > G | E218G | p.E235G | Taillandier et al. 2001 | adult | E218G/A382S | 3.6 | 7 | + | |
| 7 | c.738G > T | R229S | p.R246S | Taillandier et al. 2000 | childhood | A159T/R229S | 4.4 | 5 | − | |
| 7 | c.746G > T | G232V | p.G249V | Fedde et al. 1996 | perinatal | G232V/N | 34.5 | 3 | + | |
| 7 | c.971A > G | K247R | p.K264R | Versailles lab January 2007 | perinatal | K247R/D361V | | | − | |
| 8 | c.797_802del | S249_H250del | p.S266_H267del | Spentchian et al. 2006 | perinatal | A106D/S249_H250del | | | | Deletion of 2 a.a. |
| 8 | c.809G > A | W253X | p.W270X | Mornet et al. 1998 | perinatal | R167W/W253X | | | na | Nonsense mutation |
| 8 | c.814C > T | R255C | p.R272C | Spentchian et al. 2006 | perinatal | R255C/T117H | | | − | |
| 8 | c.815G > T | R255L | p.R272L | Spentchian et al. 2003 | perinatal | R255L/c.662delG | | | − | |
| 8 | c.815G > A | R255H | p.R272H | Brun-Heath et al. 2005 | infantile | R255H/R255H | 6.8 | 16 | − | |
| 8 | c.824T > C | L258P | p.L275P | Orimo et al. 2002 | childhood | L258P/A160T | 3.3 | 4 | − | |
| 8 | c.853_854insGATC | Y268X | p.Y285X | Michigami et al. 2005 | perinatal | c1559delT/Y268X | | | na | Nonsense mutation |
| IVS8 | c.862 + 5G > A | | | Taillandier et al. 1999 | infantile | c.862 + 5G > A/c.862 + | | | na | Affects splicing |
| | | | | | | 5G > A | | | | |
| 9 | c.865C > T | L272F | p.L289F | Sugimoto et al. 1998 | infantile | L272F/? | 50 | 8 | − | |
| 9 | c.871G > A | E274K | p.E291K | Mornet et al. 1998 | infantile | E174K/E274K | 8.3 | 1 | − | |
| 9 | c.871G > T | E274X | p.E291X | Taillandier et al. 2000 | perinatal | A94T/E274X | | | − | Nonsense mutation |
| 9 | c.874C > A | P275T | p.P292T | Brun-Heath et al. 2005 | infantile | P275T/A16V | 4.0 | 16 | + | |
| 9 | c.876_881delAGGGGA | G276_D277del | | Spentchian et al. 2003 | perinatal | G276_D277del/ | | | na | |
| | | | | | | c.962delG | | | | |
| 9 | c.880G > T | D277Y | p.D294Y | Taillandier et al. 2001 | infantile | A159T/D277Y | | | − | |
| 9 | c.881A > C | D277A | p.D294A | Henthorn et al. 1992 | infantile | R54C/D277A | 0 | 17 | − | |
| 9 | c.883A > G | M278V | p.M295V | Mornet et al. 2001 | childhood | E174K/M278V | | | − | |
| 9 | c.884T > C | M278T | p.M295T | Brun-Heath et al. 2005 | perinatal | M278T/R206W | 8.5 | 16 | − | |
| 9 | c.885G > A | M278I | p.M295I | Michigami et al. 2005 | perinatal | M278I/c.1559delT | | | − | |
| 9 | c.889T > G | Y280D | p.Y297D | Brun-Heath et al. 2005 | childhood | R119H/Y280D | 1.3 | 16 | − | |
| 9 | c.892G > A | E281K | p.E298K | Orimo et al. 1994 | infantile | E281K/1559delT | | | − | |
| 9 | c.896T > C | L282P | p.L299P | Versailles lab October 2003 | infantile | L282P/L282P | 9.7 | 15 | − | |
| 9 | c.917A > T | D289V | p.D306V | Taillandier et al. 1999 | infantile | D289V/D289V | 0 | 12 | − | |
| 9 | c.919C > T | P290S | p.P307S | Versailles lab October 2004 | infantile | P290S/M450T | | | + | |
| 9 | c.920C > T | P290L | p.P307L | Versailles lab July 2006 | childhood | P290L/S164L | | | | |
| 9 | c.928_929delTC | | | Brun-Heath et al. 2005 | perinatal | T394A/c.928_929delTC | | | | Frameshift mutation |
| 9 | c.931G > A | E294K | p.E311K | Spentchian et al. 2003 | perinatal | E294K/c.388_391delGTAA | | | − | |
| 9 | c.962delG | | | Spentchian et al. 2003 | perinatal | G276_D277del/ | | | na | Frameshift mutation |
| | | | | | | c.962delG | | | | |
| 9 | c.976G > C | G309R | p.G326R | Litmanovitz et al. 2002 | perinatal | G309R/E274K | | | + | |
| 9 | c.981_983delCTT | F310del | p.F327del | Orimo et al. 1997 | infantile | F310del/c.1559delT | ~10 | 15 | + | Amino acid deletion |
| 9 | c.979T > G | F310C | p.F327C | Mornet et al. 2001 | perinatal | T117N/F310C | | | + | |
| 9 | c.979_980TT > | F310G | p.F327G | Taillandier et al. 2001 | adult | E174K/F310G | | | + | |
| | GG | | | | | | | | | |
| 9 | c.979T > C | F310L | p.F327L | Ozono et al. 1996 | infantile | F310L/G439R | 72 | 9 | + | |
| 9 | c.982T > A | F311L | p.F328L | Michigami et al. 2005 | perinatal non- | F311L/T83M | ~10 | 15 | + | |
| | | | | | lethal | | | | | |
| IVS9 | c.997 + 2T > A | | | Taillandier et al. 2000 | perinatal | c.997 + 2T > A/C472S | | | na | Affects splicing |
| IVS9 | c.997 + 2T > G | | | Brun-Heath et al. 2005 | perinatal | c.997 + 2T > G/c.997 + | | | | Affects splicing |
| | | | | | | 2T > G | | | | |
| IVS9 | c.997 + 3A > C | | | Mornet et al. 1998 | perinatal | c.997 + 3A > C/c.997 + | | | na | Affects splicing |
| | | | | | | 3A > C | | | | |
| IVS9 | c.998-1G > T | | | Taillandier et al. 2001 | perinatal | E174K/c.998- | | | na | Affects splicing |
| | | | | | | 1G > T | | | | |
| 10 | c.1001G > A | G317D | p.G334D | Greenberg et al. 1993 | perinatal | G317D/G317D | 0 | 10 | − | |
| 10 | c.1015G > A | G322R | p.G339R | Mumm et al. 2002 | perinatal | G322R/A159T | | | − | |
| 10 | c.1016G > A | G322E | p.G339E | Versailles lab October 2004 | infantile | G322E/V111M | | | − | |
| 10 | c.1042G > A | A331T | p.A348T | Taillandier et al. 2000 | infantile | E174K/A331T | 33.2 | 5 | − | |
| 10 | c.1044_1055del | L332_A335del | p.L349_A352del | Spentchian et al. 2006 | perinatal | L332_A335del/ | | | | Deletion of 4 a.a. |
| | | | | | | G474R | | | | |
| 10 | c.1062G > C | E337D | p.E354D | Souka et al. 2002 | perinatal | I195F/E337D | | | + | |
| 10 | c.1064A > C | M338T | p.M355T | Versailles lab October 2004 | perinatal | G204V/M338T | | | − | |
| 10 | c.1065G > A | M338I | p.M355I | Versailles lab. January | infantile | M338I/R374C | | | − | |
| | | | | 2008 | | | | | | |
| 10 | c.1101_1103delCTC | S351del | p.S368del | Versailles lab October 2004 | perinatal | c.1101_1103delCTC/ | | | | Deletion of 1 a.a. |
| | | | | | | T372I | | | | |
| 10 | c.1112C > T | T354I | p.T371I | Baumgartner-Sigl et al. | infantile | M209T/T354I | | | − | |
| | | | | 2007 | | | | | | |
| 10 | c.1120G > A | V357M | p.V374M | Versailles lab October 2004 | adult | V357M/E281K | | | + | |
| 10 | c.1130C > T | A360V | p.A377V | Mornet et al. 2001 | perinatal | A360V/A360V | | | + | |
| 10 | c.1133A > T | D361V | p.D378V | Henthorn et al. 1992 | infantile | E174K/D361V | 1.2 | 3 | + | |
| 10 | c.1142A > G | H364R | p.H381R | Taillandier et al. 2000 | infantile | A23V/H364R | | | + | |
| 10 | c.1144G > A | V365I | p.V382I | Goseki-Sone et al. | childhood | F310L/V365I | 0 | 11 | + | |
| | | | | 1998 | | | | | | |
| 10 | c.1166C > T | T372I | p.T389I | Versailles lab October 2004 | perinatal | T372I/S351del | | | − | |
| 10 | c.1171C > T | R374C | p.R391C | Zurutuza et al. 1999 | childhood | E174K/R374C | 10.3 | 1 | − | |
| 10 | c.1172G > A | R374H | p.R391H | Orimo et al. 2002 | childhood | R374H/? | 3.7 | 14 | − | |
| 10 | c.1172delC | | | Taillandier et al. 1999 | infantile | M45L/c.1172delC | | | na | Frameshift mutation |
| 10 | c.1175G > C | G375A | p.G392A | Versailles lab. January | perinatal | G375A/R119C | | | − | |
| | | | | 2008 | | | | | | |
| 10 | c.1182T > C | I378T | p.I395T | Versailles lab July 2006 | perinatal | I378T/E174K | | | | |
| 11 | c.1195G > T | A382S | p.A399S | Taillandier et al. 2001 | adult | E218G/A382S | | | − | |
| 11 | c.1196C > T | A382V | p.A399V | Spentchian et al. 2006 | adult | A382V/A16V | | | − | |
| 11 | c.1199C > T | P383L | p.P400L | Spentchian et al. 2006 | infantile | P383L/P383L | | | + | |
| 11 | c.1214_1215delCA | | | Versailles lab July 2006 | adult | c.1214_1215delCA/ | | | | Frameshift mutation |
| | | | | | | E174K | | | | |
| 11 | c.1216_1219delGACA | | | Brun-Heath et al. 2005 | perinatal | c.1216_1219delGACA/? | | | | |
| 11 | c.1217A > G | D389G | p.D406G | Taillandier et al. 2000 | odonto. | D389G/R433H | 14.9 | 5 | + | |
| 11 | c.1228T > C | F393L | p.F410L | Versailles lab October 2004 | infantile | F393L/E174K | | | − | |
| 11 | c.1231A > G | T394A | p.T411A | Brun-Heath et al. 2005 | perinatal | T394A/c.926_927delTC | 0.3 | 16 | − | |
| 11 | c.1240C > A | L397M | p.L414M | Mumm et al. 2002 | perinatal | L397M/D277A | | | − | |
| 11 | c.1250A > G | N400S | p.N417S | Sergi et al. 2001 | perinatal | N400S/c.648 + | 3 | unp. | + | |
| | | | | | | 1G > A | | | | |
| 11 | c.1256delC | | | Taillandier et al. 2000 | perinatal | c.1256delC/? | | | na | Frameshift mutation |
| 11 | c.1258G > A | G403S | p.G420S | Glaser et al. 2004 | perinatal | G403S/G403S | 0.4 | unp. | − | |
| 11 | c.1268T > C | V406A | p.V423A | Taillandier et al. 2001 | perinatal | A99T/V406A | 15.7 | 2 | − | |
| 11 | c.1270G > A | V407M | p.V424M | Versailles lab January 2007 | adult | V407M/V407M | | | − | |
| 11 | c.1276G > T | G409C | p.G426C | Mochizuki et al. 2000 | infantile | K207A/G409C | 18.5 | 15 | − | |
| 11 | c.1277G > A | G409D | p.G426D | Mumm et al. 2002 | childhood | G409D/E174K | | | − | |
| 11 | c.1282C > T | R411X | p.R428X | Taillandier et al. 1999 | perinatal | R411X/R411X | | | na | Nonsense mutation |
| 11 | c.1283G > C | R411P | p.R428P | Spentchian et al. 2006 | perinatal | R411P/c.997 + | | | − | |
| | | | | | | 2T > A | | | | |
| 11 | c.1285G > A | E412K | p.E429K | Versailles lab July 2006 | odonto. | E412K/? | | | | |
| 11 | c.1306T > C | Y419H | p.Y436H | Henthorn et al. 1992 | childhood | A16V/Y419H | | | na | |
| 12 | c.1333T > C | S428P | p.S445P | Mornet et al. 1998 | infantile | S428P/? | 2.1 | 1 | − | |
| 12 | c.1349G > A | R433H | p.R450H | Taillandier et al. 2000 | odonto. | D389G/R433H | | | − | |
| 12 | c.1348C > T | R433C | p.R450C | Mornet et al. 1998 | infantile | R433C/R433C | 4.0 | 1 | − | |
| 12 | c.1354G > A | E435K | p.E452K | Spentchian et al. 2003 | perinatal | A94T/E435K | | | + | |
| 12 | c.1361A > G | H437R | p.H454R | Versailles lab October 2003 | childhood | E174K/H437R | | | + | |
| 12 | c.1363G > A | G438S | p.G455S | Draguet et al. 2004 | adult | G438S/G474R | | | − | |
| 12 | c.1364G > A | G438D | p.G455D | Versailles lab January 2007 | perinatal | G438D/G438D | | | − | |
| 12 | c.1366G > T | G439W | p.G456W | Versailles lab October 2003 | childhood | G439W/? | | | + | |
| 12 | c.1366G > A | G439R | p.G456R | Ozono et al. 1996 | infantile | G439R/? | 1.5 | unp. | + | |
| 12 | c.1375G > A | V442M | p.V459M | Taillandier et al. 2000 | infantile | A34V/V442M | | | + | |
| 12 | c.1375G > T | V442L | p.V459L | Versailles lab October 2004 | perinatal | V442L/E435K | | | − | |
| 12 | c.1396C > T | P449L | p.P466L | Versailles lab October 2003 | perinatal | P449L/? | | | + | |
| 12 | c.1400T > C | M450T | p.M467T | Versailles lab October 2004 | infantile | M450T/P290S | | | − | |
| 12 | c.1402G > A | A451T | p.A468T | Spentchian et al. 2003 | perinatal | A451T/A451T | | | + | |
| 12 | c.1417G > A | G456S | p.G473S | Mornet et al. 1998 | perinatal | A23V/G456S | | | + | |
| 12 | c.1426G > A | E459K | p.E476K | Taillandier et al. 1999 | perinatal | A94T/E459K | | | + | |
| 12 | c.1427A > G | E459G | p.E476G | Mornet et al. 2001 | perinatal | E459G/E459G | | | + | |
| 12 | c.1433A > T | N461I | p.N478I | Taillandier et al. 2000 | childhood | N461I/N | 1.1 | 3 | − | |
| 12 | c.1444_1445insC | | | Brun-Heath et al. 2005 | perinatal | c.1444_1445insC/ | | | | Frameshift mutation |
| | | | | | | G317D | | | | |
| 12 | c.1456G > C | C472S | p.C489S | Taillandier et al. 2000 | perinatal | C472S/c.997 + | 9.4 | 5 | − | |
| | | | | | | 2T > A | | | | |
| 12 | c.1468A > T | I473F | p.I490F | Lia-Baldini et al. 2001 | adult | I473F/? | 37.1 | 3 | − | |
| 12 | c.1471G > A | G474R | p.G491R | Mornet et al. 1998 | perinatal | G112R/G474R | | | − | |
| 12 | c.1471delG | | | Brun-Heath et al. 2005 | odonto | c.1471delG/R119H | | | | Frameshift mutation |
| 12 | c.1559delT | | | Orimo et al. 1994 | infantile | E281K/c.1559delT | 28 | 18 | na | Frameshift mutation |

Large deletions

| | | | |
|---|---|---|---|
| deletion of | Spentchian et al. 2006 | perinatal | homozygote |
| exons 3-5 | | | |
| deletion | Spentchian et al. 2006 | infantile | compound |
| of exon | heterozygote | | |
| 12 (3′ part) | with S164L | | |

### Spacer

Without being limited to this theory, it is believed that the Fc fragment used in the bone targeted sALP fusion protein presented in Examples below acts as a spacer which allows the protein to be more efficiently folded since expression of sTNALP-Fc-D10 was higher than that of sTNALP-D10 (see Example 2 below). One possible explanation is that the introduction of the Fc fragment alleviates the repulsive forces caused by the presence of the highly negatively charges D10 sequence added at the C-terminus of the tested sALP sequence.

Useful spacers for the present invention include polypeptides comprising a Fc, and hydrophilic and flexible polypeptides able to alleviate the repulsive forces caused by the presence of the highly negatively charged D10 sequence added at the C-terminus of the sALP sequence. In specific embodiment the spacer alleviates the steric hindrance preventing two sALP domains from two sALP monomers from interacting with each other to constitute the minimal catalytically active entity.

### Fragment Crystallizable Region (Fc) Fragments

Useful Fc fragments for the present invention include FC fragments of IgG that comprise the hinge, and the CH2 and CH3 domains. IgG-1, IgG-2, IgG-3, IgG-3 and IgG-4 for instance can be used.

### Negatively Charged Peptide

The negatively charged peptide according to the present invention may be a poly-aspartate or poly-glutamate selected from the group consisting of D10 to D16 or E10 to E16.

In specific embodiments, the bone targeted sALP fusion proteins of the present invention are associated so as to form dimers or tetramers.

Without being limited to this particular theory, in specific embodiments of the invention using a polypeptide comprising a Fc as a spacer, dimers are presumably constituted of two bone targeted sALP monomers covalently linked through the two disulfide bonds located in the hinge regions of the two Fc fragments. In this dimeric configuration the steric hindrance imposed by the formation of the interchain disulfide bonds are presumably preventing the association of sALP domains to associate into the dimeric minimal catalytically active entity present in normal cells.

Without being limited to this particular theory, it is believed that in its tetrameric structure, the association of the fusion proteins would involve one sALP domain from one dimer and another one from another dimer. The steric hindrance presumably preventing two sALP domains from the same Fc-joined dimer from interacting with each other to constitute the minimal catalytically active entity could eventually be relieved by inserting a longer spacer than the Fc described in Examples presented herein between the sALP fragment and the polyaspartate or polyglutamate fragment.

The bone targeted sALP may further optionally comprise one or more additional amino acids 1) downstream from the poly-aspartate or poly-glutamate; and/or 2) between the poly-aspartate and the Fc fragment; and/or 3) between the spacer such as the Fc fragment and the sALP fragment. This is the case for instance when the cloning strategy used to produce the bone targeting conjugate introduces exogenous amino acids in these locations. However the exogenous amino acids should be selected so as not to provide an additional GPI anchoring signal. The likelihood of a designed sequence being cleaved by the transamidase of the host cell can be predicted as described by Ikezawa (Ikezawa 2002).

The present invention also encompasses the fusion protein as post-translationally modified such as by glycolisation including those expressly mentioned herein, acetylation, amidation, blockage, formylation, gamma-carboxyglutamic acid hydroxylation, methylation, phosphorylation, pyrrolidone carboxylic acid, and sulfatation.

The term “recombinant protein” is used herein to refer to a protein encoded by a genetically manipulated nucleic acid inserted into a prokaryotic or eukaryotic host cell. The nucleic acid is generally placed within a vector, such as a plasmid or virus, as appropriate for the host cell. Although Chinese Hamster Ovary (CHO) cells have been used as a host for expressing the conjugates of the present invention in the Examples presented herein, a person of ordinary skill in the art will understand that a number of other hosts may be used to produce recombinant proteins according to methods that are routine in the art. Representative methods are disclosed in Maniatis, et al. Cold Springs Harbor Laboratory (1989). “Recombinant cleavable protein” as used herein is meant to refer to a recombinant protein that may be cleaved by a host's enzyme so as to produce a secreted/soluble protein. Without being so limited HEK293 cells, PerC6, Baby hamster Kidney cells can also be used.

As used herein the terminology “conditions suitable to effect expression of the polypeptide” is meant to refer to any culture medium that will enable production of the fusion protein of the present invention. Without being so limited, it includes media prepared with a buffer, bicarbonate and/or HEPES, ions like chloride, phosphate, calcium, sodium, potassium, magnesium, iron, carbon sources like simple sugars, amino acids, potentially lipids, nucleotides, vitamins and growth factors like insulin; regular commercially available media like alpha-MEM, DMEM, Ham's-F12 and IMDM supplemented with 2-4 mM L-glutamine and 5% Fetal bovine serum; regular commercially available animal protein free media like Hyclone™ SFM4CHO, Sigma CHO DHFR-, Cambrex POWER™ CHO CD supplemented with 2-4 mM L-glutamine. These media are desirably prepared without thymidine, hypoxanthine and L-glycine to maintain selective pressure allowing stable protein-product expression.

Without being so limited, host cells useful for expressing the fusion of the present invention include L cell, C127 cells, 3T3 cells, CHO cells, BHK cells, COS-7 cells or Chinese Hamster Ovary (CHO) cell. Particular CHO cells of interest for expressing the fusion protein of the present invention include CHO-DG44 and CHO/dhfr⁻ also referred to as CHO duk⁻. This latter cell line is available through the American Type Culture Collection (ATCC number CRL-9096).

The term “bone tissue” is used herein to refer to tissue synthesized by osteoblasts composed of an organic matrix containing mostly collagen and mineralized by the deposition of hydroxyapatite crystals.

The fusion proteins comprised in the bone delivery conjugates of the present invention are useful for therapeutic treatment of bone defective conditions by providing an effective amount of the fusion protein to the bone. The fusion proteins are provided in the form of pharmaceutical compositions in any standard pharmaceutically acceptable carriers, and are administered by any standard procedure, for example by intravenous injection.

As used herein the terminology “HPP phenotype” is meant to refer to any one of rickets (defect in growth plate cartilage), osteomalacia, elevated blood and/or urine levels of inorganic pyrophosphate (PPᵢ), phosphoethanolamine (PEA), or pyridoxal 5′-phosphate (PLP), seizure, bone pains, calcium pyrophosphate dihydrate crystal deposition (CPPD) in joints leading to chondrocalcinosis and premature death. Without being so limited, a HPP phenotype can be documented by growth retardation with a decrease of long bone length (such as femur, tibia, humerus, radius, ulna), a decrease of the mean density of total bone and a decrease of bone mineralization in bones such as femur, tibia, ribs and metatarsi, and phalange, a decrease in teeth mineralization, a premature loss of deciduous teeth (e.g., aplasia, hypoplasia or dysplasia of dental cementum). Without being so limited, correction or prevention of bone mineralization defect may be observed by one or more of the following: an increase of long bone length, an increase of mineralization in bone and/or teeth, a correction of bowing of the legs, a reduction of bone pain and a reduction of CPPD crystal deposition in joints.

As used herein the terminology “correct” in the expression “correct a hypophosphatasia phenotype” is meant to refer to any partial or complete reduction of a pre-existing HPP phenotype. Similarly the terminology “prevent” in the expression “prevent a hypophosphatasia phenotype” is meant to refer to any delay or slowing in the development of a HPP phenotype or any partial or complete avoidance of the development of a HPP phenotype.

As used herein the term “subject” is meant to refer to any mammal including human, mice, rat, dog, cat, pig, cow, monkey, horse, etc. In a particular embodiment, it refers to a human.

As used herein, the term “subject in need thereof” in a method of administering a compound of the present invention is meant to refer to a subject that would benefit from receiving a compound of the present invention. In specific embodiments, it refers to a subject that already has at least one HPP phenotype or to a subject likely to develop at least one HPP phenotype or at least one more HPP phenotype. In another embodiment it further refers to a subject that has aplasia, hypoplasia or dysplasia of dental cementum or a subject likely to develop aplasia, hypoplasia or dysplasia of dental cementum.

As used herein “a subject likely to develop at least one HPP phenotype” is a subject having at least one loss-of-function mutation in the gene (ALPL).

As used herein “a subject likely to develop aplasia, hypoplasia or dysplasia of dental cementum” is a subject having HPP or a periodontal disease due to a bacterial infection. Periodontal disease due to a bacterial infection may induce alteration of cementum which may lead to exfoliation of teeth.

### Route of Administration

Bone targeted sALPs of the present invention can be administered by routes such as orally, nasally, intravenously, intramuscularly, subcutaneously, sublingually, intrathecally, or intradermally. The route of administration can depend on a variety of factors, such as the environment and therapeutic goals. As used herein, subjects refer to animals such as humans in which prevention, or correction of bone mineralization defect characterizing HPP or other phenotypes associated with HPP or prevention or correction of defective cementum is desirable.

By way of example, pharmaceutical composition of the invention can be in the form of a liquid, solution, suspension, pill, capsule, tablet, gelcap, powder, gel, ointment, cream, nebulae, mist, atomized vapor, aerosol, or phytosome. For oral administration, tablets or capsules can be prepared by conventional means with pharmaceutically acceptable excipients such as binding agents, fillers, lubricants, disintegrants, or wetting agents. The tablets can be coated by methods known in the art. Liquid preparations for oral administration can take the form of, for example, solutions, syrups, or suspension, or they can be presented as a dry product for constitution with saline or other suitable liquid vehicle before use. Dietary supplements of the invention also can contain pharmaceutically acceptable additives such as suspending agents, emulsifying agents, non-aqueous vehicles, preservatives, buffer salts, flavoring, coloring, and sweetening agents as appropriate. Preparations for oral administration also can be suitably formulated to give controlled release of the active ingredients.

Enteric coatings can further be used on tablets of the present invention to resist prolonged contact with the strongly acidic gastric fluid, but dissolve in the mildly acidic or neutral intestinal environment. Without being so limited, cellulose acetate phthalate, Eudragit™ and hydroxypropyl methylcellulose phthalate (HPMCP) can be used in enteric coatings of pharmaceutical compositions of the present invention. Cellulose acetate phthalate concentrations generally used are 0.5-9.0% of the core weight. The addition of plasticizers improves the water resistance of this coating material, and formulations using such plasticizers are more effective than when cellulose acetate phthalate is used alone. Cellulose acetate phthalate is compatible with many plasticizers, including acetylated monoglyceride; butyl phthalybutyl glycolate; dibutyl tartrate; diethyl phthalate; dimethyl phthalate; ethyl phthalylethyl glycolate; glycerin; propylene glycol; triacetin; triacetin citrate; and tripropionin. It is also used in combination with other coating agents such as ethyl cellulose, in drug controlled-release preparations.

### Dosage

Any amount of a pharmaceutical composition can be administered to a subject. The dosages will depend on many factors including the mode of administration and the age of the subject. Typically, the amount of bone targeted ALP of the invention contained within a single dose will be an amount that effectively prevent, delay or correct bone mineralization defect in HPP without inducing significant toxicity. As used herein the term “therapeutically effective amount” is meant to refer to an amount effective to achieve the desired therapeutic effect while avoiding adverse side effects. Typically, bone targeted sALPs in accordance with the present invention can be administered to subjects in doses ranging from 0.001 to 500 mg/kg/day and, in a more specific embodiment, about 0.1 to about 100 mg/kg/day, and, in a more specific embodiment, about 0.2 to about 20 mg/kg/day. The allometric scaling method of Mahmood et al. (Mahmood et al. 2003) can be used to extrapolate the dose from mice to human. The dosage will be adapted by the clinician in accordance with conventional factors such as the extent of the disease and different parameters from the patient.

The therapeutically effective amount of the bone targeted sALP may also be measured directly. The effective amount may be given daily or weekly or fractions thereof. Typically, a pharmaceutical composition of the invention can be administered in an amount from about 0.001 mg up to about 500 mg per kg of body weight per day (e.g., 0.05, 0.01, 0.1, 0.2, 0.3, 0.5, 0.7, 0.8, 1 mg, 2 mg, 3 mg, 4 mg, 5 mg, 10 mg, 15 mg, 20 mg, 30 mg, 50 mg, 100 mg, or 250 mg). Dosages may be provided in either a single or multiple dosage regimens. For example, in some embodiments the effective amount is a dose that ranges from about 0.1 to about 100 mg/kg/day, from about 0.2 mg to about 20 mg of the bone targeted sALP per day, about 1 mg to about 10 mg of the bone targeted sALP per day, from about 0.07 mg to about 210 mg of the bone targeted sALP per week, 1.4 mg to about 140 mg of the bone targeted sALP per week, about 0.3 mg to about 300 mg of the bone targeted sALP every three days, about 0.4 mg to about 40 mg of the bone targeted sALP every other day, and about 2 mg to about 20 mg of the bone targeted sALP every other day.

These are simply guidelines since the actual dose must be carefully selected and titrated by the attending physician based upon clinical factors unique to each patient or by a nutritionist. The optimal daily dose will be determined by methods known in the art and will be influenced by factors such as the age of the patient as indicated above and other clinically relevant factors. In addition, patients may be taking medications for other diseases or conditions. The other medications may be continued during the time that a bone targeted sALP is given to the patient, but it is particularly advisable in such cases to begin with low doses to determine if adverse side effects are experienced.

### Carriers/Vehicles

Preparations containing a bone targeted sALP may be provided to patients in combination with pharmaceutically acceptable sterile aqueous or non-aqueous solvents, suspensions or emulsions. Examples of non-aqueous solvents are propylene glycol, polyethylene glycol, vegetable oil, fish oil, and injectable organic esters. Aqueous carriers include water, water-alcohol solutions, emulsions or suspensions, including saline and buffered medical parenteral vehicles including sodium chloride solution, Ringer's dextrose solution, dextrose plus sodium chloride solution, Ringer's solution containing lactose, or fixed oils. Intravenous vehicles may include fluid and nutrient replenishers, electrolyte replenishers, such as those based upon Ringer's dextrose, and the like.

In yet another embodiment, the pharmaceutical compositions of the present invention can be delivered in a controlled release system. In one embodiment polymeric materials including polylactic acid, polyorthoesters, cross-linked amphipathic block copolymers and hydrogels, polyhydroxy butyric acid and polydihydropyrans can be used (see also Smolen and Ball, Controlled Drug Bioavailability, Drug product design and performance, 1984, John Wiley & Sons; Ranade and Hollinger, Drug Delivery Systems, pharmacology and toxicology series, 2003, 2^{nd} edition, CRRC Press), in another embodiment, a pump may be used (Saudek et al., 1989, N. Engl. J. Med. 321: 574).

The fusion proteins of the present invention could be in the form of a lyophilized powder using appropriate excipient solutions (e.g., sucrose) as diluents.

Further, the nucleotide segments or proteins according to the present invention can be introduced into individuals in a number of ways. For example, osteoblasts can be isolated from the afflicted individual, transformed with a nucleotide construct according to the invention and reintroduced to the afflicted individual in a number of ways, including intravenous injection. Alternatively, the nucleotide construct can be administered directly to the afflicted individual, for example, by injection. The nucleotide construct can also be delivered through a vehicle such as a liposome, which can be designed to be targeted to a specific cell type, and engineered to be administered through different routes.

The fusion proteins of the present invention could also be advantageously delivered through gene therapy. Useful gene therapy methods include those described in WO06060641A2, U.S. Pat. No. 7,179,903 and WO0136620A2 to Genzyme using for instance an adenovirus vector for the therapeutic protein and targeting hepatocytes as protein producing cells.

A “gene delivery vehicle” is defined as any molecule that can carry inserted polynucleotides into a host cell. Examples of gene delivery vehicles are liposomes, biocompatible polymers, including natural polymers and synthetic polymers; lipoproteins; polypeptides; polysaccharides; lipopolysaccharides; artificial viral envelopes; metal particles; and bacteria, or viruses, such as baculovirus, adenovirus and retrovirus, bacteriophage, cosmid, plasmid, fungal vectors and other recombination vehicles typically used in the art which have been described for expression in a variety of eukaryotic and prokaryotic hosts, and may be used for gene therapy as well as for simple protein expression. “Gene delivery,” “gene transfer,” and the like as used herein, are terms referring to the introduction of an exogenous polynucleotide (sometimes referred to as a “transgene”) into a host cell, irrespective of the method used for the introduction. Such methods include a variety of well-known techniques such as vector-mediated gene transfer (e.g., viral infection/transfection, or various other protein-based or lipid-based gene delivery complexes) as well as techniques facilitating the delivery of “naked” polynucleotides (such as electroporation, “gene gun” delivery and various other techniques used for the introduction of polynucleotides). The introduced polynucleotide may be stably or transiently maintained in the host cell. Stable maintenance typically requires that the introduced polynucleotide either contains an origin of replication compatible with the host cell or integrates into a replicon of the host cell such as an extrachromosomal replicon (e.g., a plasmid) or a nuclear or mitochondrial chromosome. A number of vectors are known to be capable of mediating transfer of genes to mammalian cells, as is known in the art and described herein.

A “viral vector” is defined as a recombinantly produced virus or viral; particle that comprises a polynucleotide to be delivered into a host cell, either in viva, ex viva or in vitro. Examples of viral vectors include retroviral vectors, adenovirus vectors, adeno-associated virus vectors such as those described in WO06002203A2, alphavirus vectors and the like. Alphavirus vectors, such as Semliki Forest virus-based vectors and Sindbis virus-based vectors, have also been developed for use in gene therapy and immunotherapy.

In aspects where gene transfer is mediated by a DNA viral vector, such as an adenovirus (Ad) or adeno-associated virus (MV), a vector construct refers to the polynucleotide comprising the viral genome or part thereof, and a transgene. Adenoviruses (Ads) are a relatively well characterized, homogenous group of viruses, including over 50 serotypes. See, e.g., International PCT Application No. WO 95/27071. Ads are easy to grow and do not require integration into the host cell genome. Recombinant Ad derived vectors, particularly those that reduce the potential for recombination and generation of wild-type virus, have also been constructed. See, International PCT Application Nos. WO 95/00655 and WO 95/11984. Vectors that contain both a promoter and a cloning site into which a polynucleotide can be operatively linked are well known in the art. Such vectors are capable of transcribing RNA in vitro or in vivo, and are commercially available from sources such as Stratagene (La Jolla, Calif.) and Promega Biotech (Madison, Wis.). In order to optimize expression and/or in vitro transcription, it may be necessary to remove, add or alter 5′ and/or 3′ untranslated portions of the clones to eliminate extra, potential inappropriate alternative translation initiation codons or other sequences that may interfere with or reduce expression, either at the level of transcription or translation.

The bone targeted sALP of the present invention may also be used in combination with at least one other active ingredient to correct a bone mineralization defect or another detrimental symptom of HPP. It may also be used in combination with at least one with at least one other active ingredient to correct cementum defect.

The term “high stringency conditions” are meant to refer to conditions enabling sequences with a high homology to bind. Without being so limited, examples of such conditions are listed In the handbook “Molecular cloning, a laboratory manual, second edition of 1989 from Sambrook et al.: 6×SSC or 6×SSPE, Denhardt's reagent or not, 0.5% SDS and the temperature used for obtaining high stringency conditions is most often in around 68° C. (see pages 9.47 to 9.55 of Sambrook) for nucleic acid of 300 to 1500 nucleotides. Although the optimal temperature to be used for a specific nucleic acid probe may be empirically calculated, and although there is room for alternatives in the buffer conditions selected, within these very well known condition ranges, the nucleic acid captured will not vary significantly. Indeed, Sambrook clearly indicates that the “choice depends to a large extent on personal preference” (see page 9.47). Sambrook specifies that the formula to calculate the optimal temperature which varies according to the fraction of guanine and cytosine in the nucleic acid probe and the length of the probe (10 to 20° C. lower than Tₘ wherein Tₘ=81.5° C.+16.6(log₁₀[Na⁺])+0.41(fraction G+C)−0.63 (% formamide−(600/l)) (see pages 9.50 and 9.51 of Sambrook).

### Kits

The present invention also relates to a kit for correcting or preventing an HPP phenotype or a cementum defect comprising a nucleic acid, a protein or a ligand in accordance with the present invention. For instance it may comprise a bone targeted composition of the present invention or a vector encoding same, and instructions to administer said composition or vector to a subject to correct or prevent a HPP phenotype. Such kits may further comprise at least one other active agent able to prevent or correct a HPP phenotype. When the kit is used to prevent or correct a HPP phenotype in a HPP subject, the kit may also further comprise at least one other active agent capable of preventing or correcting any other detrimental symptoms of HPP. In addition, a compartmentalized kit in accordance with the present invention includes any kit in which reagents are contained in separate containers. Such containers include small glass containers, plastic containers or strips of plastic or paper. Such containers allow the efficient transfer of reagents from one compartment to another compartment such that the samples and reagents are not cross-contaminated and the agents or solutions of each container can be added in a quantitative fashion from one compartment to another.

More specifically, in accordance with a first aspect of the present invention, there is provided a bone targeted alkaline phosphatase comprising a polypeptide having the structure: Z-sALP-Y-spacer-X-Wn-V, wherein sALP is the extracellular domain of the alkaline phosphatase; wherein V is absent or is an amino acid sequence of at least one amino acid; X is absent or is an amino acid sequence of at least one amino acid; Y is absent or is an amino acid sequence of at least one amino acid; Z is absent or is an amino acid sequence of at least one amino acid; and Wn is a polyaspartate or a polyglutamate wherein n=10 to 16.

In a specific embodiment, the sALP comprises amino acid residues 23-508 of SEQ ID NO: 15. In another specific embodiment, the sALP consists of amino acid residues 23-512 of SEQ ID NO: 15. In another specific embodiment, the sALP comprises amino acid residues 23-508 of SEQ ID NO: 18. In another specific embodiment, the sALP consists of amino acid residues 23-512 of SEQ ID NO: 18. In another specific embodiment, the sALP comprises amino acid residues 18-498 of SEQ ID NO: 16. In another specific embodiment, the sALP consists of amino acid residues 18-502 of SEQ ID NO: 16. In another specific embodiment, the sALP comprises amino acid residues 18-498 of SEQ ID NO: 19. In another specific embodiment, the sALP consists of amino acid residues 18-502 of SEQ ID NO: 19. In another specific embodiment, the sALP comprises amino acid residues 18-498 of SEQ ID NO: 19. In another specific embodiment, the sALP consists of amino acid residues 18-502 of SEQ ID NO: 19. In another specific embodiment, the sALP comprises amino acid residues 18-498 of SEQ ID NO: 8. In another specific embodiment, the sALP consists of amino acid residues 18-502 of SEQ ID NO: 8.

In another specific embodiment, the spacer comprises a fragment crystallizable region (Fc). In another specific embodiment, the Fc comprises a CH2 domain, a CH3 domain and a hinge region. In another specific embodiment, the Fc is a constant domain of an immunoglobulin selected from the group consisting of IgG-1, IgG-2, IgG-3, IgG-3 and IgG-4. In another specific embodiment, the Fc is a constant domain of an immunoglobulin IgG-1. In another specific embodiment, the Fc is as set forth in SEQ ID NO: 3. In another specific embodiment, Wn is a polyaspartate. In another specific embodiment, n=10. In another specific embodiment, Z is absent. In another specific embodiment, Y is two amino acid residues. In another specific embodiment, Y is leucine-lysine. In another specific embodiment, X is 2 amino acid residues. In another specific embodiment, X is aspartate-isoleucine. In another specific embodiment, V is absent. In another specific embodiment, the polypeptide is as set forth in SEQ ID NO: 4.

In another specific embodiment, the bone targeted alkaline phosphatase comprises the polypeptide in a form comprising a dimer. In another specific embodiment, the bone targeted alkaline phosphatase comprises the polypeptide in a form of a tetramer.

In another specific embodiment, the bone targeted alkaline phosphatase is in a pharmaceutically acceptable carrier. In another specific embodiment, the pharmaceutically acceptable carrier is a saline. In another specific embodiment, the bone targeted alkaline phosphatase is in a lyophilized form. In another specific embodiment, the bone targeted alkaline phosphatase is in a daily dosage of about 0.2 to about 20 mg/kg. In another specific embodiment, the bone targeted alkaline phosphatase is in a dosage of about 0.6 to about 60 mg/kg for administration every three days. In another specific embodiment, the bone targeted alkaline phosphatase is in a weekly dosage of about 1.4 to about 140 mg/kg. In another specific embodiment, the bone targeted alkaline phosphatase is in a weekly dosage of about 0.5 mg/kg.

More specifically, in accordance with another aspect of the present invention, there is provided an isolated nucleic acid comprising a sequence that encodes the polypeptide of the present invention.

In accordance with another aspect of the present invention, there is provided an isolated nucleic acid consisting of a sequence that encodes the polypeptide of the present invention. More specifically, in accordance with another aspect of the present invention, there is provided an isolated nucleic acid comprising a sequence as set forth in SEQ ID NO: 17.

In accordance with another aspect of the present invention, there is provided a recombinant expression vector comprising the nucleic acid of the present invention. More specifically, in accordance with another aspect of the present invention, there is provided a recombinant adeno-associated virus vector comprising the nucleic acid of the present invention. More specifically, in accordance with another aspect of the present invention, there is provided an isolated recombinant host cell transformed or transfected with the vector of the present invention.

In accordance with another aspect of the present invention, there is provided a method of producing the bone targeted alkaline phosphatase of the present invention, comprising culturing the host cell of the present invention, under conditions suitable to effect expression of the bone targeted alkaline phosphatase and recovering the bone targeted alkaline phosphatase from the culture medium.

In a specific embodiment, the host cell is a L cell, C127 cell, 3T3 cell, CHO cell, BHK cell, COS-7 cell or a Chinese Hamster Ovary (CHO) cell. In another specific embodiment, the host cell is a Chinese Hamster Ovary (CHO) cell. In a specific embodiment, the host cell is a CHO-DG44 cell.

In accordance with another aspect of the present invention, there is provided a kit comprising the bone targeted alkaline phosphatase of the present invention, and instructions to administer the polypeptide to a subject to correct or prevent a hypophosphatasia (HPP) phenotype.

In accordance with another aspect of the present invention, there is provided a kit comprising the bone targeted alkaline phosphatase of the present invention, and instructions to administer the polypeptide to a subject to correct or prevent aplasia, hypoplasia or dysplasia of dental cementum.

In accordance with another aspect of the present invention, there is provided a method of using the bone targeted alkaline phosphatase of the present invention, for correcting or preventing at least one hypophosphatasia (HPP) phenotype, comprising administering a therapeutically effective amount of the bone targeted alkaline phosphatase to a subject in need thereof, whereby the at least one HPP phenotype is corrected or prevented in the subject.

In a specific embodiment, the subject has at least one HPP phenotype. In another specific embodiment, the subject is likely to develop at least one HPP phenotype. In another specific embodiment, the at least one HPP phenotype comprises HPP-related seizure. In another specific embodiment, the at least one HPP phenotype comprises premature loss of deciduous teeth. In another specific embodiment, the at least one HPP phenotype comprises incomplete bone mineralization. In another specific embodiment, incomplete bone mineralization is incomplete femoral bone mineralization. In another specific embodiment, incomplete bone mineralization is incomplete tibial bone mineralization. In another specific embodiment, incomplete bone mineralization is incomplete metatarsal bone mineralization. In another specific embodiment, incomplete bone mineralization is incomplete ribs bone mineralization. In another specific embodiment, the at least one HPP phenotype comprises elevated blood and/or urine levels of inorganic pyrophosphate (PPi). In another specific embodiment, the at least one HPP phenotype comprises elevated blood and/or urine levels of phosphoethanolamine (PEA). In another specific embodiment, the at least one HPP phenotype comprises elevated blood and/or urine levels of pyridoxal 5′-phosphate (PLP). In another specific embodiment, the at least one HPP phenotype comprises inadequate weight gain. In another specific embodiment, the at least one HPP phenotype comprises rickets. In another specific embodiment, the at least one HPP phenotype comprises bone pain. In another specific embodiment, the at least one HPP phenotype comprises calcium pyrophosphate dihydrate crystal deposition. In another specific embodiment, the at least one HPP phenotype comprises aplasia, hypoplasia or dysplasia of dental cementum. In another specific embodiment, the subject in need thereof has infantile HPP. In another specific embodiment, the subject in need thereof has childhood HPP. In another specific embodiment, the subject in need thereof has perinatal HPP. In another specific embodiment, the subject in need thereof has adult HPP. In another specific embodiment, the subject in need thereof has odontohypophosphatasia HPP.

In accordance with another aspect of the present invention, there is provided a method of using the bone targeted alkaline phosphatase of the present invention, for correcting or preventing aplasia, hypoplasia or dysplasia of dental cementum, comprising administering a therapeutically effective amount of the bone targeted alkaline phosphatase to a subject in need thereof, whereby aplasia, hypoplasia or dysplasia of dental cementum is corrected or prevented in the subject.

In a specific embodiment, the administering comprises transfecting a cell in the subject with a nucleic acid encoding the alkaline phosphatase. In another specific embodiment, the transfecting the cell is performed in vitro such that the bone targeted alkaline phosphatase is expressed and secreted in an active form and administered to the subject with said cell. In another specific embodiment, the administering comprises subcutaneous administration of the bone targeted alkaline phosphatase to the subject. In another specific embodiment, the administering comprises intravenous administration of the bone targeted alkaline phosphatase to the subject.

In accordance with another aspect of the present invention, there is provided the bone targeted alkaline phosphatase of the present invention, for use in correcting or preventing at least one HPP phenotype.

In accordance with another aspect of the present invention, there is provided the bone targeted alkaline phosphatase of the present invention, for use in correcting or preventing aplasia, hypoplasia or dysplasia of dental cementum.

In accordance with another aspect of the present invention, there is provided a use of the bone targeted alkaline phosphatase of the present invention, in the making of a medicament.

In accordance with another aspect of the present invention, there is provided a use of the bone targeted alkaline phosphatase of the present invention, for correcting or preventing at least one HPP phenotype.

In accordance with another aspect of the present invention, there is provided a use of the bone targeted alkaline phosphatase of the present invention, for correcting or preventing aplasia, hypoplasia or dysplasia of dental cementum.

Other objects, advantages and features of the present invention will become more apparent upon reading of the following non-restrictive description of specific embodiments thereof, given by way of example only with reference to the accompanying drawings.

### BRIEF DESCRIPTION OF THE DRAWINGS

In the appended drawings:

FIG. 1 presents the design and schematic structure of the bone targeted ALP of the present invention exemplified by hsTNALP-FcD10. Panel A presents a schematic representation of the complete primary translation product of the human tissue non-specific alkaline phosphatase gene (TNALP) including the N-terminal signal peptide and the transient membrane-anchored signal for GPI-addition. Panel B presents the primary translation product of the fusion protein. Panel C presents the primary translation product lacking the cleavable TNALP signal peptide;

FIG. 2 presents the protein sequence for hTNALP-FcD10 ((SEQ ID NO: 1), including the N-terminal peptide signal-17 first aa), wherein the hTNALP portion (SEQ ID NO: 2) is italicized including the peptide signal portion shown italicized and underlined, and the Fc fragment is underlined (SEQ ID NO: 3);

FIG. 3 presents the protein sequence for the hsTNALP-FcD10 used in Examples presented herein (SEQ ID NO: 4) (without the N-terminal peptide signal) wherein the hsTNALP portion (SEQ ID NO: 5) is italicized, and the Fc fragment is underlined (SEQ ID NO: 3). Double underlined asparagine (N) residues correspond to putative N-glycosylation sites and bold amino acid residues (LK & DI) correspond to linkers between hsTNALP and Fc, and Fc and D10 domains respectively. These linkers are derived from endonuclease restriction sites introduced during cDNA engineering;

FIG. 4 graphically presents the comparative expression of sTNALP-D10 and sTNALP-FcD10 in CHO-DG44 cells;

FIG. 5 presents sTNALP-FcD10 purification on protein-A Sepharose molecular sieve chromatography on Sephacryl™ 3-300 as well as SDS-PAGE analysis of purified sTNALP-FcD10 under reducing (DTT+) and non reducing (DTT−) conditions. It also presents a schematized version of sTNALP-FcD10. The protein purified by Protein A-Sepharose™ affinity chromatography was analyzed by SDS-PAGE and bands stained with Sypro™ Ruby. Main species of sTNALP-FcD10 migrated with an apparent molecular mass of 90,000 Da under reducing conditions and 200,000 Da under non reducing conditions;

FIG. 6 presents the position of the papain cleavage site in sTNALP-FcD10;

FIG. 7 presents a non denaturing SEC-HPLC analysis of sTNALP-FcD10 on TSK-Gel G3000WXL column. Plain curve: papain digested sample. -X- curve: identical sample incubated in the same conditions without papain (control);

FIG. 8 presents a SDS-PAGE analysis of sTNALP-FcD10 incubated with or without papain showing which fragment is responsible for which band on the gel. Analysis was performed under reducing (+DTT) or non reducing (−DTT) conditions;

FIG. 9 presents an in vitro binding assay. sTNALP-FcD10 and bovine kidney tissue non specific alkaline phosphatase were compared in the reconstituted mineral binding assay as described in Example 2. Total activity is the sum of the enzymatic activity recovered in the free and bound fractions. Total activity was found to be 84% and 96% of initial amount of enzymatic activity introduced in each set of assays for the bovine and sTNALP-FcD10 forms of enzyme, respectively. Results are the average of two bindings;

FIG. 10 presents pharmacokinetic and distribution profiles of sTNALP-FcD10 in serum, tibia and muscle of adult WT mice. Concentrations of sTNALP-FcD10 in serum, tibia and muscle, is expressed in μg/g tissue (wet weight) after a single bolus intravenous injection of 5 mg/kg in adult WT mice;

FIG. 11 presents pharmacokinetic profile of sTNALP-FcD10 serum concentration in newborn WT mice. Serum concentrations of sTNALP-FcD10 as a function of time after a single i.p. (panel A) or s.c. (panel B) injection of 3.7 mg/kg in (1 day old) newborn WT mice;

FIG. 12 presents the predicted pharmacokinetic profile of sTNALP-FcD10 in serum. Predicted maximal (Cmax) and minimal (Cmin) circulating steady-state levels of sTNALP-FcD10 after repeated (every 24 hrs) subcutaneous injections of 10 mg/Kg in newborn mice;

FIG. 13 presents the experimentally tested pharmacokinetic profile of sTNALP-FcD10 in the serum of newborn mice. Measured minimal (Cmin) circulating steady-state levels of sTNALP-FcD10 24 h after the last subcutaneous injections of 10 mg/Kg in newborn mice. Homo: homozygous, hetero: heterozygous;

FIG. 14 presents short-term (15 days), low dose (1 mg/Kg), efficacy results in terms of sTNALP-FcD10 serum concentrations in treated Akp2^{-/-} mice. sTNALP-FcD10 serum concentrations at day 16 of mice treated for 15 days with daily s.c. injections of 1 mg/kg sTNALP-FcD10;

FIG. 15 presents short-term (15 days), low dose (1 mg/Kg), efficacy results in terms of serum PPi concentrations in treated Akp2^{-/-} mice. Measurement of serum PPi concentrations. A low dose of 1 mg/kg was sufficient to normalize PPi levels in ERT-treated mice;

FIG. 16 presents short-term (15 days), low dose (1 mg/Kg), efficacy results in terms of physeal morphology in treated Akp2^{-/-} mice. Goldner's trichrome staining of the growth plates of WT, untreated and treated Akp2^{-/-} mice. The proximal tibial growth plates (physes) showed excessive widening of the hypertrophic zone in both sTNALP-FcD10 and vehicle injected in Akp2^{-/-} mice, consistent with early rickets. However, physeal morphology seemed less disturbed in the animals treated with sTNALP-FcD10;

FIG. 17 presents short-term (15 days), low dose (1 mg/Kg), efficacy results in terms of physeal hypertrophic area size of treated Akp2^{-/-} mice. Size of the hypertrophic area of the growth plate is expressed as a percentage of the total growth plate area. Note the normalization of the hypertrophic area in the treated mice;

FIG. 18 presents short-term (15 days), high dose (8.2 mg/Kg), efficacy results in terms of body weight in treated Akp2−/− mice. Effect of sTNALP-FcD10 on body weight;

FIG. 19 presents short-term (15 days), high dose (8.2 mg/Kg), efficacy results in terms of long bone length in treated Akp2^{-/-} mice. Effect of sTNALP-FcD10 on femur and tibial length (measurements done at day 16);

FIG. 20 presents short-term (15 days), high dose (8.2 mg/Kg), efficacy results in terms of sTNALP-FcD10 serum concentration in treated Akp2^{-/-} mice. sTNALP-FcD10 serum concentrations at day 16 of mice treated for 15 days with daily s.c. injections of 8.2 mg/kg sTNALP-FcD10;

FIG. 21 presents short-term (15 days), high dose (8.2 mg/Kg), efficacy results in terms of mineralization of bones in treated Akp2^{-/-} mice. X-ray analysis of feet, rib cages and hind limbs of Akp2^{-/-} mice (16 days) and a Faxitron™ image distribution table. Feet and rib cages were classified as severe, moderate or healthy to take into account the extent of the bone mineralization defects. Legs were simply classified as abnormal (at least one defect) or healthy (no visible defect);

FIG. 22 presents short-term (15 days), high dose (8.2 mg/Kg), efficacy results in terms of defects in teeth in treated Akp2^{-/-} mice. Histological analysis of teeth of Akp2^{-/-} mice injected vehicle or sTNALP-FcD10 and wild-type mice. Thin sections were prepared and stained as described in Millan et al. PDL=Peridontal ligament;

FIG. 23 presents long-term (52 days), high dose (8.2 mg/Kg), efficacy results in terms of survival in treated Akp2^{-/-} mice. Long-term survival of Akp2^{-/-} mice treated with sTNALP-FcD10 compared to the early demise of Akp2^{-/-} treated only with control vehicle;

FIG. 24 presents long-term (52 days), high dose (8.2 mg/Kg), efficacy results in terms of size, mobility and appearance in treated Akp2^{-/-} mice. Treatment normalizes size, mobility and appearance of treated Akp2^{-/-} mice. Untreated mouse from the same litter is shown for comparison;

FIG. 25 presents long-term (52 days), high dose (8.2 mg/Kg), efficacy results in terms of mineralization and length of bones in treated Akp2^{-/-} mice. X-ray images of the metatarsal bones of 46 and 53-days old treated Akp2^{-/-} mice in comparison with WT mice;

FIG. 26 presents long-term (52 days), high dose (8.2 mg/Kg), efficacy results in terms of sTNALP-FcD10 serum concentration in treated Akp2^{-/-} mice. sTNALP-FcD10 serum concentrations at day 53 of mice treated for 52 days with daily s.c. injections of 8.2 mg/kg sTNALP-FcD10;

FIG. 27 presents A) survival curves of Akp2^{-/-} mice receiving sTNALP-FcD10 at doses of either 4.3 mg/kg daily (Tx-1) or 15.2 mg/kg every 3 days (Tx-3) or 15.2 mg/kg every week (Tx-7) and B) median survival for each of these regimen. Survival of the treated mice was compared to the survival of mice injected vehicle;

FIG. 28 presents A) survival curves of Akp2^{-/-} mice receiving sTNALP-FcD10 at doses of 8.2 mg/kg daily (RTx) starting at day 15 after birth and B) median survival for treated and vehicle injected mice. Survival of the treated mice is compared to the survival of mice injected vehicle (RVehicle);

FIG. 29 presents the effects on body weight of daily 8.2 mg/kg doses of sTNALP-FcD10 injected to Akp2^{-/-} mice (RTx) starting at day 15 after birth. Daily body weights are compared to that of vehicle-injected Akp2^{-/-} mice (RVehicle) or wild-type littermates (WT);

FIG. 30 presents an alignment of various ALPs established by CLUSTAL™ W (1.82) multiple sequence alignment, namely a bovine TNALP sequence (SEQ ID NO: 6); a cat TNALP sequence (SEQ ID NO: 7), a human TNALP sequence (SEQ ID NO: 8), a mouse TNALP sequence (SEQ ID NO: 9), a rat TNALP sequence (SEQ ID NO: 10) and a partial dog TNALP sequence (SEQ ID NO: 11) wherein the nature of the first 22 amino acid residues are unknown; a human IALP (SEQ ID NO: 12) (Accession no: NP_{—}001622), a human GCALP (SEQ ID NO: 13) (Accession no: P10696), and a human PLALP (SEQ ID NO: 14) (Accession no: NP_{—}112603). “*” denotes that the residues in that column are identical in all sequences of the alignment, “:” denotes that conserved substitutions have been observed, and “.” denotes that semi-conserved substitutions have been observed. A consensus sequence derived from this alignment (SEQ ID NO: 15) is also presented wherein x is any amino acid;

FIG. 31 presents an alignment of TNALPs from various species established by CLUSTAL™ W (1.82) multiple sequence alignment, namely the bovine sequence (SEQ ID NO: 6); the cat sequence (SEQ ID NO: 7), the human sequence (SEQ ID NO: 8), the mouse sequence (SEQ ID NO: 9), the rat sequence (SEQ ID NO: 10) and a partial dog sequence (SEQ ID NO: 11) wherein the nature of the first 22 amino acid residues are unknown. “*” denotes that the residues in that column are identical in all sequences of the alignment, “:” denotes that conserved substitutions have been observed, and “.” denotes that semi-conserved substitutions have been observed. A consensus sequence derived from this alignment (SEQ ID NO: 16) is also presented wherein x is any amino acid; and

FIG. 32 presents the nucleic acid sequence (SEQ ID NO:17) encoding the polypeptide sequence described in FIG. 1.

### DESCRIPTION OF ILLUSTRATIVE EMBODIMENTS

Examples provided below present the first successful treatment of TNALP knockout (Akp2−/−) mice using subcutaneous injections of a recombinant form of ALP. Akp2^{-/-} mice recapitulate the severe, often lethal, infantile form of Hypophosphatasia.

The well-described TNSALP-homozygous null murine model which mirrors many of the skeletal and biochemical abnormalities associated with infantile HPP was used. Mice were treated with a novel soluble recombinant form of human TNSALP engineered at its carboxy-terminus to contain both a spacer in the form of the crystalline fragment (Fc) region of human IgG-1 fused to a bone targeting sequence composed of ten sequential aspartic acid (D10) residues. It was shown that relative to native TNSALP purified from kidney, the modified recombinant form of the enzyme, binds hydroxyapatite much more avidly, while retaining its enzymatic activity. Treatment with the recombinant TNSALP of the present invention surprisingly normalized plasma PPi levels, and improved mineralization of the feet thoraces, hind limbs and dentition of homozygous null mice when compared to mice who received the vehicle alone. The treatment was also shown to prolong survival, with near radiographic normalization of the skeletal phenotype.

In addition to its beneficial in vivo therapeutic effect, it was surprisingly discovered that the recombinant active form of the modified enzyme which contains a spacer is expressed at higher levels than its recombinant counterpart lacking such spacer. In addition, it was demonstrated that the enzyme functions as a tetramer.

The present invention is illustrated in further details by the following non-limiting examples.

### Example 1

### Expression and Purification of Recombinant sTNALP-FcD10

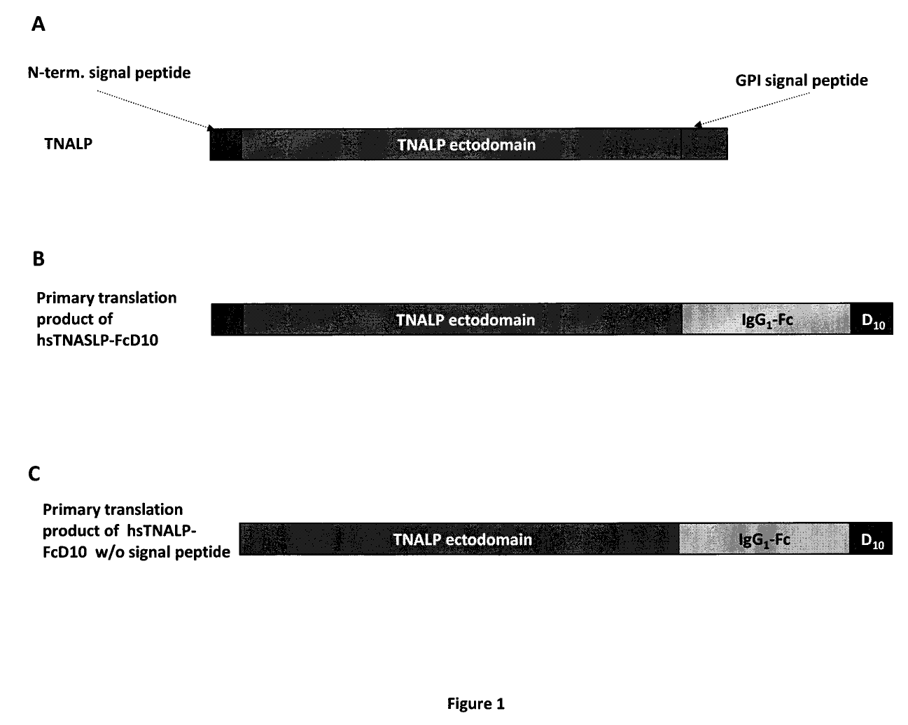

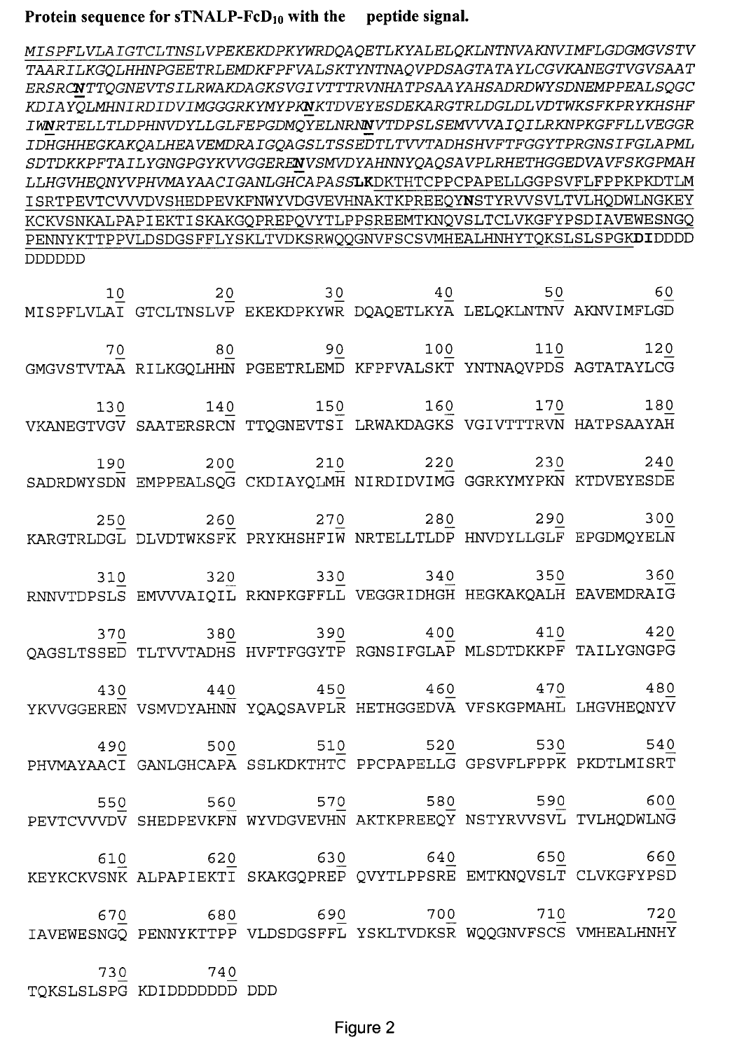

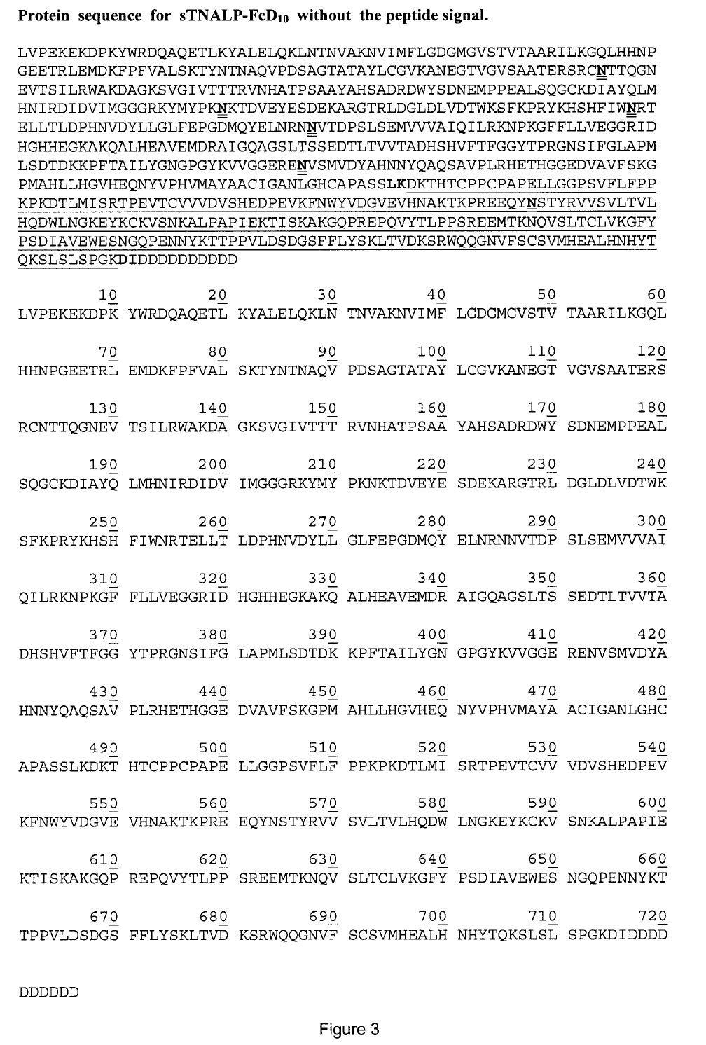

In order to facilitate the expression and purification of recombinant TNALP, the hydrophobic C-terminal sequence that specifies GPI-anchor attachment in TNALP was eliminated to make it a soluble secreted enzyme (Di Mauro et al. 2002). The coding sequence of the TNALP ectodomain was also extended with the Fc region of the human IgG (γ1 form (IgG1), Swiss-Prot P01857). This allowed rapid purification of the recombinant enzyme on Protein A chromatography and surprisingly, its increased expression. Furthermore, to target the recombinant TNALP to bone tissue, a deca-aspartate (D10) sequence was attached to the C-terminal of the Fc region. This chimeric form of TNALP, designated sTNALP-FcD10, retains full enzymatic activity both when assayed at pH 9.8 using the artificial substrate p-nitrophenylphosphate and when assayed at pH 7.4 using inorganic pyrophosphate (PPi), as the physiological substrate. As in the naturally occurring form of TNALP the N-terminal signal peptide is cleaved off during the cotranslational translocation of the protein across the rough endoplasmic reticulum. Its design and structure is schematically illustrated in FIG. 1. The amino acid sequence of the fusion protein (including the signal peptide) is shown in FIG. 2. The amino acid sequence of the fusion protein as secreted (i.e. without the signal peptide) is shown in FIG. 3.

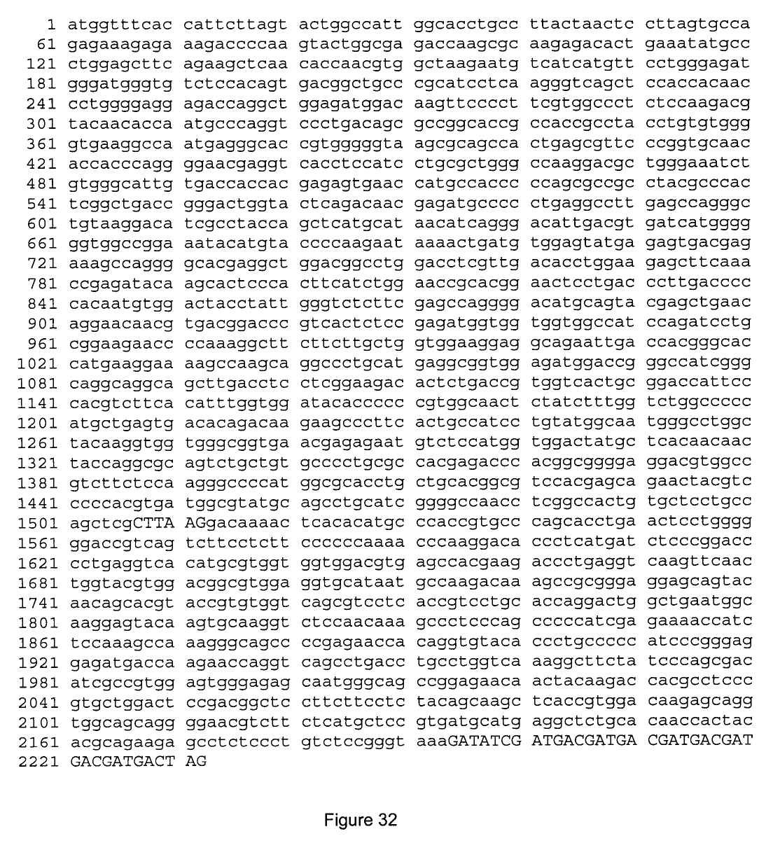

The method that was used to construct this fusion protein is as follows. The cDNA encoding the fusion protein (See FIG. 32) was inserted in the pIRES vector (Clontech™) in the first multiple cloning site located upstream of the IRES using NheI and BamHI endonuclease restriction sites. The dihydrofolate reductase (DHFR) gene was inserted in the second multiple cloning site located downstream of the IRES using SmaI and XbaI endonuclease restriction sites. The resulting vector was transfected into Chinese Hamster Ovary (CHO-DG44) cells lacking both DHFR gene alleles (Urlaub et al. 1983, obtained from Dr Lawrence A. Chasin, Columbia University) using the Lipofectamine™ transfection kit (Invitrogen). Two days after transfection, media was changed and the cells were maintained in a nucleotide free medium (IMDM supplemented with 5% dialyzed FBS) for 15 days to isolate stable transfectants for plaque cloning.

Cells from the three best clones or the five originally selected growing in the nucleotide-free medium were pooled and further cultivated in media (IMDM+5% dialyzed FBS) containing increasing concentration of methotrexate (MTX). Cultures resistant to 50 nM MTX were further expanded in Cellstacks™ (Corning) containing IMDM medium supplemented with 5% FBS. Upon reaching confluency, the cell layer was rinsed with Phosphate Buffered Saline (PBS) and cells were incubated for three additional days with IMDM containing 3.5 mM sodium butyrate to increase protein expression. At the end of the culture the concentration of sTNALP-FcD10 in the spent medium was 3.5 mg/l as assessed by measuring TNALP enzymatic activity.

Levels of sALP-FcD10 in spent medium were quantified using a colorimetric assay for ALP activity where absorbance of released p-nitrophenol is proportional to the reaction products. The reaction occurred in 100 μl of ALP buffer (20 mM Bis Tris Propane (HCl) pH 9, 50 mM NaCl, 0.5 mM MgCl₂, and 50 μM ZnCl₂) containing 10 μl of diluted spent medium and 1 mM pNPP. The latter compound was added last to initiate the reaction. Absorbance was recorded at 405 nm every 45 seconds over 20 minutes using a spectrophotometric plate reader. sTNALP-FcD10 catalytic activity, expressed as an initial rate, was assessed by fitting the steepest slope for 8 sequential values. Standards were prepared with varying concentrations of sALP-FcD10, and ALP activity was determined as above. The standard curve was generated by plotting Log of the initial rate as a function of the Log of the standard concentrations. sTNALP-FcD10 concentration in the different samples was read from the standard curve using their respective ALP absorbance. Activity measures were transformed into concentrations of sALP-FcD10 by using a calibration curve obtained by plotting the activity of known concentrations of purified recombinant enzyme.

Culture supernatant was then concentrated and dialyzed against PBS using tangential flow filtration and loaded on MabSelect SuRe™ column (GE Health Care) equilibrated with 150 mM NaCl, 10 mM sodium PO₄. Bound proteins were eluted with 50 mM Tris pH 11, pH 11.0 buffer. Collected fractions were adjusted to pH 8-9 with 200 mM Tris-HCl pH 5.5. Fractions containing most of the eluted material were dialyzed against 150 mM NaCl, 25 mM sodium PO₄ pH 7.4 buffer containing 0.1 mM MgCl₂, 20 μM ZnCl₂ and filtered on a 0.22 μm (Millipore, Millex-GP™) membrane under sterile conditions. The overall yield of the purification procedure was 50% with a purity above 95% as assessed by Sypro™ ruby stained SDS-PAGE. Purified sTNALP-FcD10 preparation was stored at 4° C. and remained stable for several months.

The following purification technique was also tested with success. Culture supernatant was concentrated and dialyzed against PBS using tangential flow filtration and loaded on Protein A-Sepharose™ column (Hi-Trap™ 5 ml, GE Health Care) equilibrated with PBS. Bound proteins were eluted with 100 mM citrate pH 4.0 buffer. Collected fractions were immediately adjusted to pH 7.5 with 1 M Tris pH 9.0. Fractions containing most of the eluted material were dialyzed against 150 mM NaCl, 25 mM sodium PO₄ pH 7.4 buffer containing 0.1 mM MgCl₂, 20 μM ZnCl₂ and filtered on a 0.22 μm (Millipore, Millex-GP™) membrane under sterile conditions. The overall yield of the purification procedure was 50% with a purity above 95% as assessed by Sypro™ ruby stained SDS-PAGE. Purified sTNALP-FcD10 preparation was stored at 4° C. and remained stable for several months.

The number of copies of the sTNALP-FcD10 gene was increased by culturing transfected CHO-DG44194 cells in the presence of increasing concentration of methotrexate. Clones of cells resistant to 100 nM methotrexate were isolated and evaluated for their capacity to produce sTNALP-FcD10 at a high yield. The best producers were adapted to culture in suspension in Hyclone Media™ SFM4CHO™ (cat #SH30549) in absence of fetal bovine serum. Cultures that maintained a high production yield under those conditions were transferred to disposable Waver™ bioreactor bags. The medium (25 L total volume) was seeded at a density of 0.4×10⁶ cells per ml. Temperature of the culture was maintained at 37° C. until the cell density reached 2×10⁶ cells/ml. The temperature was then reduced to 30° C. and the culture was supplemented with 125 ml of CHO generic feed (Sigma, C1615). Those conditions were found to slow down cell division and increase product secretion in the culture medium. These conditions were maintained for 6 days before harvesting cell culture supernatant containing secreted sTNALP-FcD10.

### Example 2

### Comparative Expression of sTNALP-D10 and sTNALP-FcD10

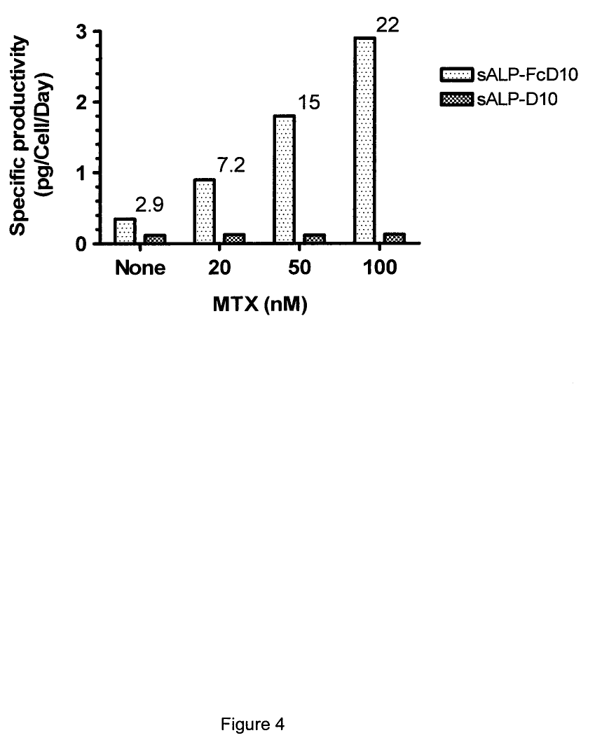

Plasmid vectors encoding either sTNALP-FcD10 or sTNALP-D10 were transfected in CHO-DG44 cells using Lipofectamine™ and grown in selective media (i.e. devoid of nucleotides) designed to promote survival of cells expressing the DHFR gene as described in Example 1 above. Stable transfectants were isolated by plaque cloning and ranked according to their level of protein expression using the alkaline phosphatase enzymatic assay also described in Example 1 above. Screening allowed the identification of one clone only for sTNALP-D10 (0.120 pg/cell/day) and five clones for sTNALP-FcD10 (0.377, 0.258, 0.203, 0.099 and 0.088 pg/cell/day). Methotrexate (MTX) gene amplification was performed as described in Example 1 above (MTX ranging from 0 to 100 mM) and allowed an 8-fold expression increase for sTNALP-FcD10 while no amplification was observed with the sTNALP-D10 cultures (see FIG. 4). Using a similar process for cell line development, it was unexpectedly found that the sTNALP-FcD10 protein was easier to express compared to sTNALP-D10 (see FIG. 4).

### Example 3

### sTNALP-FcD10 Characterization

sTNALP-FcD10 was first purified on Protein-A Sepharose™ and was analyzed on SDS-PAGE under reducing and non-reducing conditions.

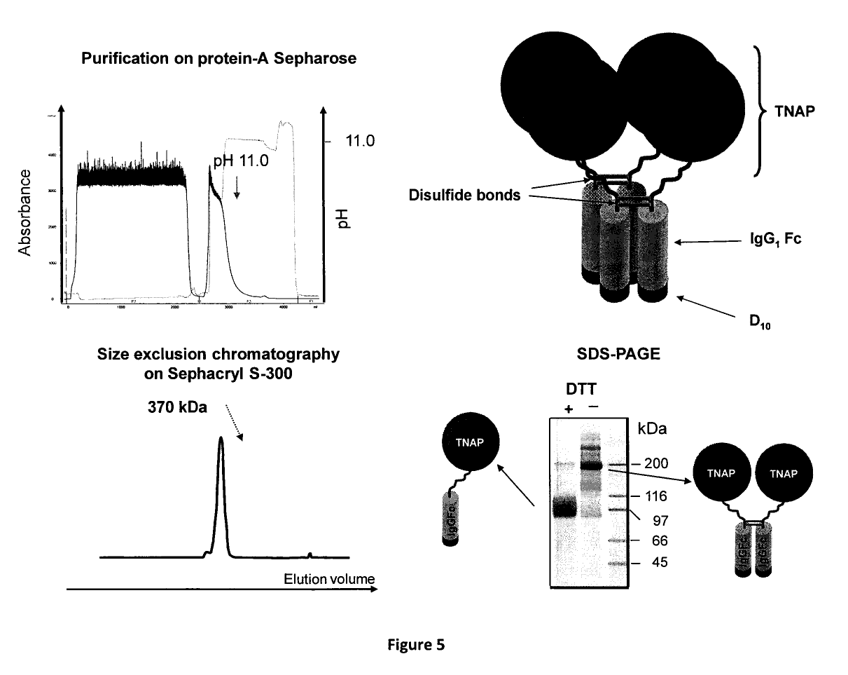

Under reducing conditions, it migrated as a broad band with an apparent molecular mass of ˜90,000 Da (DTT+ in FIG. 5). Digestion with peptide N-Glycosidase F (PNGAse F) reduced the apparent molecular mass of the protein to about 80,000 which closely approximates the calculated mass of 80,500 Da for the non-glycosylated sTNALP-FcD10 monomer shown in FIG. 1. Soluble TNALP in serum, like TNALP present as a GPI anchored protein on the outer surface of osteoblasts, is a highly glycosylated protein with carbohydrates comprising ≦20% of the total mass of the enzyme (Farley & Magnusson 2005). Although the specific carbohydrate structures on TNALP have not been identified, sequence studies indicate that the enzyme possesses five putative sites for N-linked glycosylation, and biochemical studies have shown evidence for both N- and O-linked carbohydrates (Nosjean et al. 1997). In agreement with these earlier observations, the electrophoretic migration of sTNALP-FcD10 and its sensitivity to PNGAse F suggests it is also a heavily N-glycosylated protein. Soluble TNALP in serum, like TNALP present as a GPI anchored protein on the outer surface of osteoblasts, is a highly glycosylated protein with carbohydrates comprising ≦20% of the total mass of the enzyme (Farley & Magnusson 2005).

When SDS-PAGE was repeated under non reducing conditions the apparent molecular mass of sTNALP-FcD10 was found to be 200,000 (DTT− in FIG. 5) consistent with that of a homodimer as in native unaltered TNALP (Milián 2006). This homodimer likely results from the formation of two disulfide bridges between two monomeric Fc regions (upper right panel, FIG. 5).

The molecular mass of purified sTNALP-FcD10 under native conditions was next evaluated using size exclusion FPLC chromatography on a column of Sephacryl™ S-300 (GE Health Care) equilibrated in 150 mM NaCl, 20 mM Tris pH 7.5 buffer. The column was previously calibrated with a standard protein kit (HMW calibration kit, GE Health care) (lower left panel, FIG. 5).

Collected chromatography fractions were assayed for alkaline phosphatase enzymatic activity and the material in each peak. Surprisingly, 78% of the material eluted at a position corresponding to proteins of 370 kDa (lower left panel, FIG. 5) suggesting a tetrameric form for the native sTNALP-FcD10 recombinant enzyme produced in CHO cells. When fractions from the Sephacryl S-300 column were tested for activity, all of the enzymatic activity was associated with the 370 kDa fraction. The remaining material was of a much higher molecular weight indicating the formation of some sTNALP-FcD10 aggregates. Both the tetrameric forms, which may not be observed on the SDS-page because the latter destroys the non covalent binding maintaining the tetramer together, and aggregate forms were resolved as sTNALP-FcD10 monomers with an apparent molecular weight of 90,000 by SDS-PAGE under reducing conditions (DTT+, lower right panel in FIG. 5) and as dimers with an apparent molecular weight of 200,000 in non reducing conditions (DTT−, lower right panel in FIG. 5). Recombinant sTNALP-FcD10 appears to consist mainly of enzymatically functional homotetramers formed by non covalent association of two sTNALP-FcD10 disulfide-linked dimers.

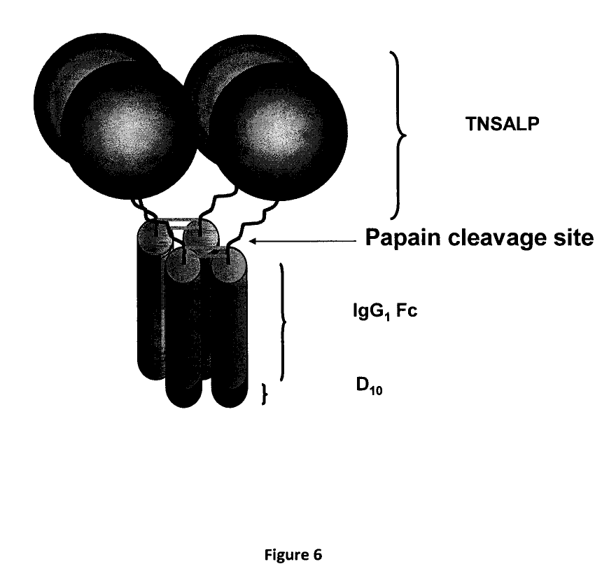

The tetrameric structure of sTNALP-FcD10 was further tested by limited papain digestion (FIGS. 6-8). This protease is known to cleave IgG heavy chains close to the hinge region and on the N-terminal side of the disulfide bonds, thereby generating whole monomeric Fab fragments and dimeric disulfide-linked Fc dimers. Digestion of sTNALP-FcD10 should thus liberate enzymatically active sTNALP dimers from the intact Fc domains (see FIG. 6).

Aliquots containing 400 μg of sTNALP-FcD10 were digested with 208 mU of papain-agarose (Sigma) in a 20 mM phosphate buffer (pH 7.0) containing 250 μM dithiothreitol. Digestion was left to proceed at 37° C. for 1 h under gentle agitation. The reaction was stopped by removing the papain-agarose beads by centrifugation. In those conditions, there was no significant loss of sTNALP-FcD10 enzymatic activity during the first 4 h of incubation. sTNALP-FcD10 incubated for one h in the presence or absence of papain-agarose was next analyzed by SEC-HPLC on a TSK-Gel G3000WXL (Tosoh Bioscience) in non denaturing conditions.

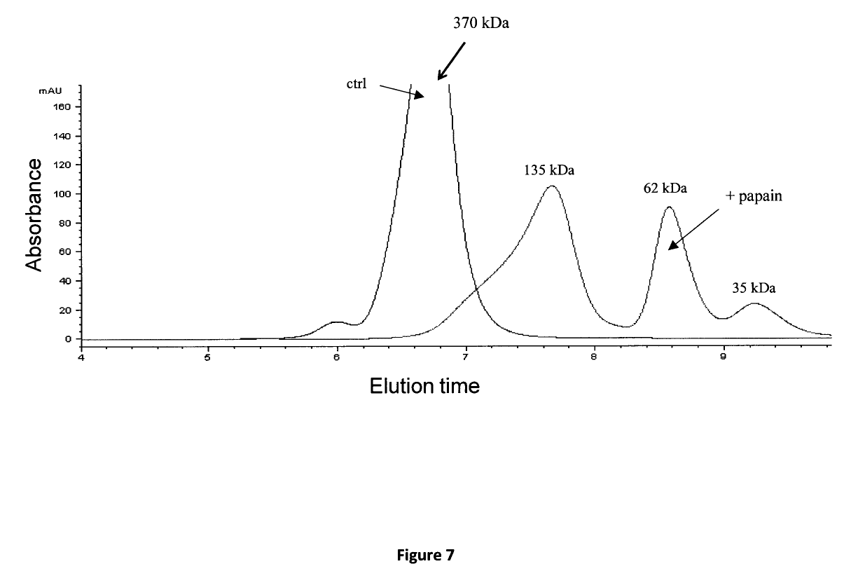

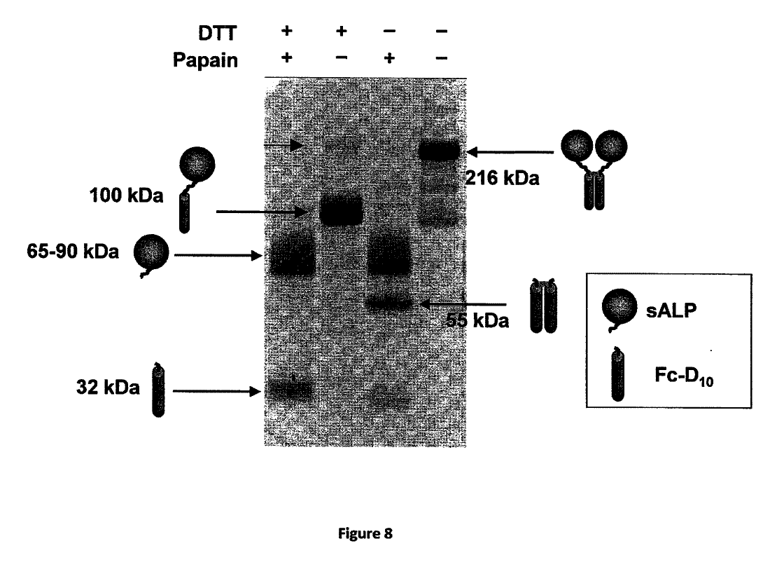

FIG. 7 shows that the main product eluting with an apparent Mr of 370 kDa was no longer observed after a 1 h papain digestion. In those conditions papain digestion generates two main fragments of 135 kDa and 62 kDa respectively. A minor peak with Mr of 35 kDa was also observed.

Under reducing SDS-PAGE conditions (DTT+, FIG. 8) the product of the non papain treated sample was resolved into a major band (102 kDa) (DTT+, papain−), which was previously shown to correspond to monomeric sTNALP-FcD10. In Western blots this band can indeed be stained with antibodies for both TNALP and the Fc domain of the IgG₁ molecule (not shown). After papain digestion this band is cleaved into two major fragments: 1) The 32 kDa band, which binds the anti-Fc but not the anti-TNALP antibody and is proposed to correspond to the FcD10 fragment; and 2) The broad and diffuse protein band (66-90 kDa) which can be stained with the anti-ALP antibody but not with anti-Fc antibody and is thus thought to correspond to TNALP ectodomain monomers. The heterogeneity of this material is presumably due to its glycosylation as it can be reduced by digestion with Peptide-N-Glycosidase F, which also decreases its apparent molecular mass to 52 kDa (results not shown).

Under non-reducing conditions (DTT−, FIG. 8), sTNALP-FcD10 incubated without papain was found to migrate in SDS-Page as a 216 kDa protein (DTT−, papain−, FIG. 8). Western blotting also demonstrates that this protein contains epitopes for both the TNALP and Fc moieties (results not shown). This molecular species was previously proposed to consist of disulfide-bonded sTNALP-FcD10 dimers. As under reducing conditions, papain cleavage under non-reducing conditions (DTT−, papain +) generates two main fragments. In Western blots, the 55 kDa fragment can be stained with the anti-Fc but not with the anti-TNALP antibody. This fragment is most probably identical to the 62 kDa species observed on SEC-HPLC in native conditions and is proposed to correspond to disulfide-bonded Fc dimers. The other major species comigrates with the major protein band (66-90 kDa) observed under reducing conditions. This is consistent with it being composed of TNALP ectodomain monomers. When analyzed by HPLC in non denaturing conditions these monomers are non-covalently associated in the enzymatically active TNALP dimers eluting from the SEC column as the 135 kDa species.

### Example 4

### Compared Affinity for Hydroxyapatite of sTNALP-FcD10 Protein and Bovine Kidney sALP

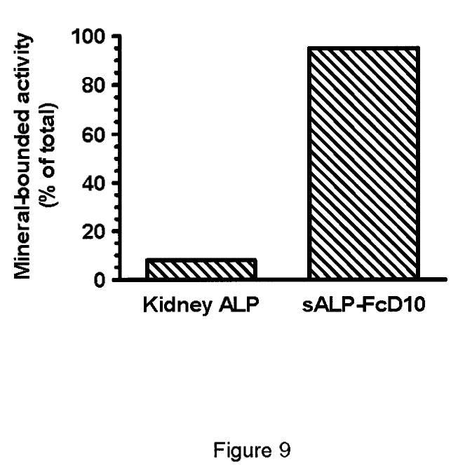

The affinity of the purified sTNALP-FcD10 protein for hydroxyapatite was also compared to that of bovine kidney (tissue non specific) soluble alkaline phosphatase (Calzyme) using the following procedure. Bovine kidney TNALP was used instead of human bone TNALP because it was commercially available. Hydroxyapatite ceramic beads (Biorad) were first solubilized in 1 M HCl and the mineral was precipitated by bringing back the solution to pH to 7.4 with 10 N NaOH. Binding to this reconstituted mineral was performed by incubating aliquots of the mineral suspension containing 750 μg of mineral with 5 μg of protein in 100 μl of 150 mM NaCl, 80 mM sodium phosphate pH 7.4, buffer. The samples were kept at 21±2° C. for 30 minutes on a rotating wheel. Mineral was spun down by low speed centrifugation and total enzymatic activity recovered in both the mineral pellet and the supernatant was measured. FIG. 9 clearly shows that sTNALP-FcD10 binds more efficiently to reconstituted hydroxyapatite mineral than bovine kidney TNALP. Furthermore, most of the recombinant sTNALP-FcD10 protein introduced in the assay was recovered by summing up the enzymatic activity recovered in both the bound and non bound fractions. This indicates that binding of the recombinant protein to the reconstituted mineral phase does not significantly alter its enzymatic activity.

### Example 5

### Mouse Model

The Akp2^{-/-} mice were created by insertion of the Neo cassette into exon VI of the mouse TNALP gene (Akp2) via homologous recombination (Narisawa et al. 1997; Fedde et al. 1999). This mutation caused the functional inactivation of the Akp2 gene and no mRNA or TNALP protein is detectable in these knockout mice (Narisawa et al. 1997). Phenotypically, the Akp2^{-/-} mice mimic severe infantile HPP. These mice have no obvious hypophosphatasia phenotype at birth, skeletal defects usually appearing at or around day 6, and worsen thereafter. They have stunted growth with rickets, develop epileptic seizures and apnea, and were reported to die between postnatal days 12-16. Like HPP patients, Akp2^{-/-} mice feature hypophosphatasemia due to global deficiency of TNALP activity, endogenous accumulation of the ALP substrates, PPi, PLP and PEA and suffer impaired skeletal matrix mineralization leading to rickets or osteomalacia (Fedde et al. 1999).

To understand how defects in alkaline phosphatase can lead to neurological manifestations of the disease in both human and mice, one has to review the role and metabolism of Vitamin B6 in the CNS. Vitamin B6 is an important nutrient that serves as a cofactor for at least 110 enzymes, including those involved in the biosynthesis of the neurotransmitters γ-aminobutyric acid (GABA), dopamine and serotonin. Vitamin B6 can be found in three free forms (or vitamers), i.e., pyridoxal (PL), pyridoxamine (PM), and pyridoxine (PN), all of which can be phosphorylated to the corresponding 5′-phosphated derivatives, PLP, PMP and PNP (Jansonius 1998). Removal of the phosphate group is a function of ALP, and primarily that of the TNALP isozyme (Whyte 2001). Since only dephosphorylated vitamers can be transported into the cells, decreased TNALP activity in Hypophosphatasia results in marked increases in plasma PLP (Whyte et al. 1985; Whyte 2001) and intracellular deficiency of PLP in peripheral tissues and the central nervous system where it leads to reduced brain levels of GABA. It has also been hypothesized that the epileptic seizures observed in these mice result from glutamic acid decarboxylase dysfunction due to shortage of PLP (Waymire et al. 1995).

Pyridoxine supplementation suppresses the epileptic seizures of Akp2^{-/-} mice but extends their lifespan only a few days, till postnatal days 18-22 (Narisawa et al. 2001). Therefore, all animals in this study (breeders, nursing moms, pups and weanlings) were given free access to a modified laboratory rodent diet 5001 with increased levels (325 ppm) of pyridoxine.

Akp2^{-/-} mice (12.5% C57BL/6-87.5% 129J hybrid background) were maintained by heterozygote breeding. Animals, breeder pairs or nursing moms with their pups, were housed in a ventilated solid bottom plastic cage equipped with an automatic watering system. All animals had free access to a modified laboratory rodent diet 5001 with 325 ppm pyridoxine (#48057, TestDiet™). Maximum allowable concentrations of contaminants in the diet (e.g. heavy metals, aflatoxin, organophosphate, chlorinated hydrocarbons, PCBs) were assured by the manufacturers. No known contaminants were present in the dietary material to influence the toxicity of the test article.

### Example 6

### Pharmacokinetics and Tissue Distribution of Injected sTNALP-FcD10 in WT Mice

### Blood Sample Collection

Blood samples were collected into heparin lithium tube (VWR, #CBD365958), put on ice for a maximum of 20 minutes before centrifugation at 2500 g for 10 min at room temperature. At least, 15 μl of plasma was transferred into 0.5 ml tube (Sarstedt, #72.699), frozen in liquid nitrogen and kept at −80° C. until assayed. If available, another ≦50 μl of plasma was transferred into 0.5 ml tube, inactivated at 65° C. for 10 minutes, frozen in liquid nitrogen and kept at −80° C. until assayed. Any remaining plasma, was pooled with the 15 μl aliquot, frozen in liquid nitrogen and kept at −80° C. until assayed.

Determination of Plasma sTNALP-FcD10

Presence of sTNALP-FcD10 in plasma samples was assessed upon completion of treatment using a colorimetric enzymatic assay. Enzymatic activity was determined using a chromogenic substrate where increase of absorbance is proportional to substrate conversion to products. The reaction was carried out in 100 μl of buffer 50 mM NaCl, 20 mM Bis Tris Propane (HCl) pH 9 buffer containing 0.5 mM MgCl₂ and 50 μM ZnCl₂ to which was added 10 μl of diluted plasma sample. The ALP substrate p-nitrophenyl was added last at a final concentration of 1 mM to initiate the reaction. The absorbance was recorded at 405 nm every 45 seconds over a twenty minutes period using a Spectramax™ 190 (Molecular devices) plate reader. sTNALP-FcD10 enzymatic activity expressed as an initial rate of reaction was assessed by fitting the steepest slope over 8 adjacent reading values. Standards were prepared with varying concentrations of test article and the enzymatic activity was determined as described above in Example 1. The standard curve was generated by plotting Log of the initial speed rate as a function of the Log of the standard quantities. sTNALP-FcD10 concentration of the different plasma samples was read directly from the standard curve using their respective enzymatic activity.

### Determination of Plasma PPi

Circulating levels of PPi were measured in serum obtained from cardiac puncture using differential adsorption on activated charcoal of UDP-D-[6-³H]glucose (Amersham Pharmacia) from the reaction product of 6-phospho[6-³H]gluconate, as previously described (Johnson et al. 1999).

Half-Life and Tissue Distribution of sTNALP-FcD10

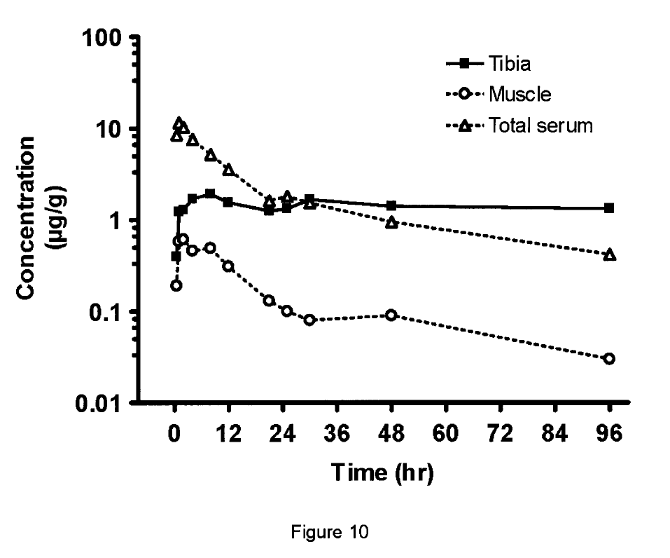

In adult WT mice, the half-life and tissue distribution of sTNALP-FcD10 injected into mice were determined. FIG. 10 summarizes its pharmacokinetics and tissue distribution after a single, bolus intravenous injection of 5 mg/kg into adult WT mice.

The half-life was 34 h in blood with an accumulation of the [¹²⁵I]-labeled sTNALP-FcD10 in bone of up to 1 μg/g of bone (wet weight). This half-life is comparable to that observed previously in unsuccessful reported clinical trials. Levels of bone-targeted material seemed quite stable, as no significant decrease in radiolabeled sTNALP-FcD10 was observed during the experiment. No accumulation of sTNALP-FcD10 was observed in muscle, as the amount of radiolabeled enzyme in that tissue decreased in parallel with that of sTNALP-FcD10 enzymatic activity in blood.

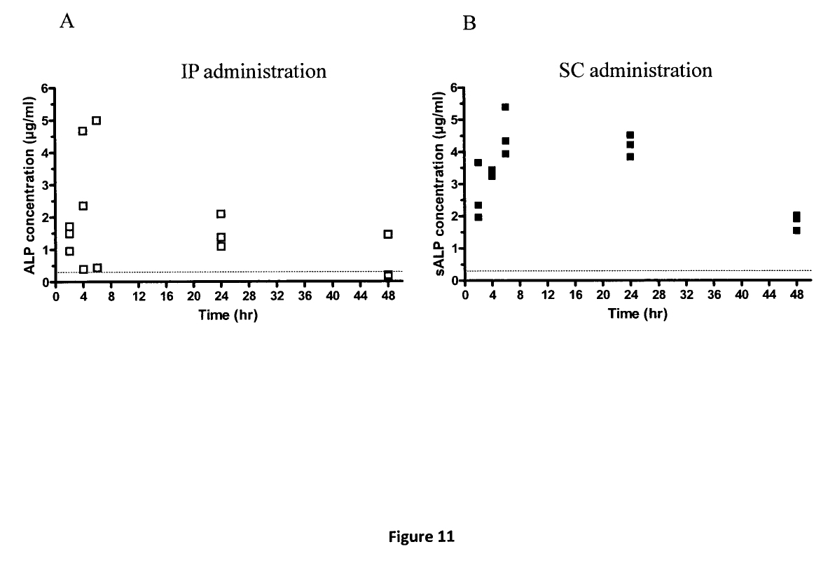

In newborn mice. Because Akp2^{-/-} mice die between days 12-16 and i.v. injection is not feasible in such young mice, the pharmacokinetic analysis of sTNALP-FcD10 in serum was repeated using the i.p. and s.c. routes in WT newborn mice using a dose of 3.7 mg/Kg. The i.p. route was found inadequate due to the high pressure in the abdominal cavity leading to unpredictable losses through the injection site (FIG. 11 A). The s.c. route was more reproducible in newborn mice, as seen in the PK experiment of FIG. 11 B. The pharmacokinetic parameters of sTNALP-FcD10 in newborn and adult mice is reported in Table 2 below.

TABLE 2
Pharmacokinetic parameters of sTNALP-
FcD10 in newborn WT mice.

Newborn

| Parameter | | s.c. | i.p. |
|---|---|---|---|
| T½ | (h) | 31 | 19 |
| Tmax | (h) | 6 | 6 |
| Cmax | (mg/L) | 5 | 3 |
| AUCinf | (mg/L/h) | 257 | 92 |

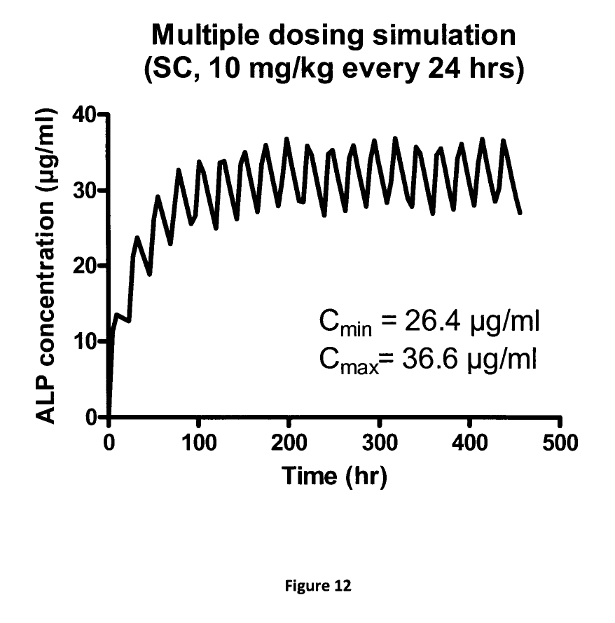

These PK data, analyzed by WinNonlin™ software (Pharsight Corporation, Mountain View, Calif.), were used to predict circulating blood levels of sTNALP-FcD10 achieved after repeated daily s.c. injections. Circulating sTNALP-FcD10 reached steady state serum concentrations oscillating between Cmin and Cmaxvalues of 26.4 and 36.6 μg/ml, respectively (FIG. 12). Steady state was achieved after 5 to 6 daily doses of 10 mg/kg.

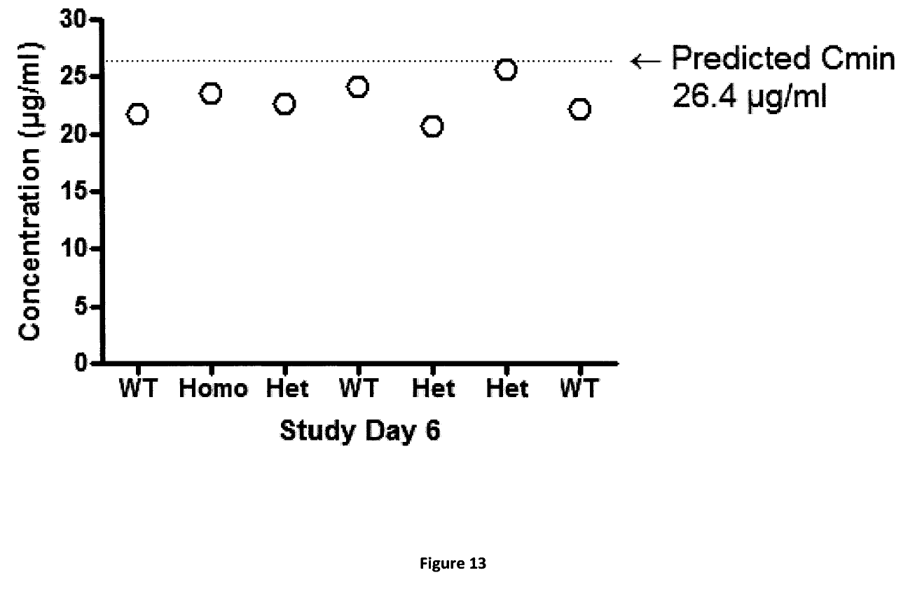

Prediction validity was tested experimentally after 5 daily injections of 10 mg/kg of sTNALP-FcD10. At the day of injection, the mice genotype could not be distinguished. It was later determined which amongst the mice tested were heterozygous or homozygous. There was no difference in the behavior of all the different genotypes. When circulating ALP activity was measured 24 h after the last injection, namely on day 6, (Cmin), good agreement was observed between experimental and predicted concentrations (FIG. 13). In these non treated 5 day old animals, serum TNALP levels were 0.58 μg/ml. These levels will decrease with age. Thus, it was calculated that the injection regimen allowed building up to steady state serum concentrations of sTNALP-FcD10 approximately 50 times higher than normal TNALP concentrations.

### Example 7

### Concentration of sTNALP-FcD10 in Adult WT Mice Bones after Bolus Intravenous Administration

A 5 mg/kg sTNALP-FcD10 dose was administered i.v. in 129J adult WT mice. The sTNALP-FcD10 concentration in bone at T=25 hours was as follows: 0.64 μg/g calvaria; 1.33 μg/g tibias; and 1.37 μg/g femurs, for a mean concentration of 1.11 μg/g. In rat, bone tissues represent 16.3% of total mass. It is expected that this percentage is also found in mice. The body weight of mice used for this experiment was 18.4 g. The calculated bone tissue weight of these mice was thus about 18.4 g×0.163=3.00 g. The calculated quantity of sTNALP-FcD10 in bone tissues was of 3.33 μg. The percentage of the injected dose in bone tissues was thus of (3.33 μg/(5 μg/g*18.4 g))*100=4%.

The sTNALP-FcD10 concentration in bone at T=96 hours was as follows: 0.83 μg/g calvaria; 1.33 μg/g tibias and 1.63 μg/g femurs, for a mean concentration of 11.26 μg/g. The body weight of mice used for this experiment was 17.8 g. The calculated bone tissue weight of these mice was thus about 17.8 g×0.163=2.90 g. The quantity of sTNALP-FcD10 in mice bone tissues was thus about 3.66 μg. The percentage of the injected dose in bone tissues was thus of (3.66 μg/(5 μg/g*17.8 g))*100=4%.

### Example 8

### Concentration of sTNALP-FcD10 in Newborn WT Mice Bones after 15 Days Bolus Subcutaneous Injection

A 4.3 mg/kg sTNALP-FcD10 dose was administered subcutaneously in 129J newborn WT mice every day for 15 days for a total administered amount of 65 mg/kg. The sTNALP-FcD10 concentration in bone at T=24 hours was as follows: 6.45 μg/g calvaria; 3.05 μg/g tibias; and 3.71 μg/g femurs, for a mean concentration of 4.40 μg/g. The body weight of mice used for this experiment was 9.83 g. The calculated bone tissue weight of these mice was thus about 9.83 g×0.163=1.60 g. The quantity of sTNALP-FcD10 in mice bone tissues at that time was thus about 7.04 μg. The percentage of the injected dose in bone tissues was thus of (7.04 μg/(65 μg/g*9.83 g))*100=1%.

The sTNALP-FcD10 concentration in bone at T=168 hours was as follows: 5.33 μg/g calvaria; 1.37 μg/g tibias; and 1.88 μg/g femurs, for a mean concentration of 2.86 μg/g. The body weight of mice used for this experiment was 14.0 g. The calculated bone tissue weight of these mice was thus about 14.0 g×0.163=2.28 g. The quantity of sTNALP-FcD10 in mice bone tissues at that time was thus about 6.52 μg. The percentage of the injected dose in bone tissues was thus of (6.52 μg/(65 μg/g*14 g))*100=0.7%. Table 3 below summarizes results of Examples 7 and 8.

TABLE 3
Mean concentration of sTNALP-FcD10 and
percentage of injected dose in bones

| | Injection | Mean concentration in | | % of injected dose in | |
|---|---|---|---|---|---|
| Experiment | regimen | bones (μg/g wet tissue) | | bones | |
| IV Bolus | 1 × 5 mg/kg | T = 25 h⁽¹⁾ | T = 96 h⁽¹⁾ | T = 25 h⁽¹⁾ | T = 96 h⁽¹⁾ |
| | (bolus) | 1.11 | 1.26 | 4% | 4% |
| SC Bolus | 15 × 4.3 mg/kg | T = 24 h⁽¹⁾ | T = 168 h⁽¹⁾ | T = 24 h⁽¹⁾ | T = 168 h⁽¹⁾ |
| over 15 days | (daily) | 4.40 | 2.86 | 1% | 0.7% |

⁽¹⁾Times indicated are from the last injection.

### Example 9

### Short-Term (15 Days) Efficacy of Low Doses (1 mg/kg) of sTNALP-FcD10 for HPP in Akp2^{-/-} Mice

Daily s.c. injection of sTNALP-FcD₁₀ were performed for 15 days in Akp2^{-/-} mice using 1 mg/kg. Treatment groups were constituted from 19 litters. Akp2^{-/-} mice received vehicle (N=13) or sTNALP-FcD10 (N=12). Controls consisted of 15 WT mice (one per litter). Controls were not submitted to injections. Blood was taken 24 h after the last injection as described in Example 6.

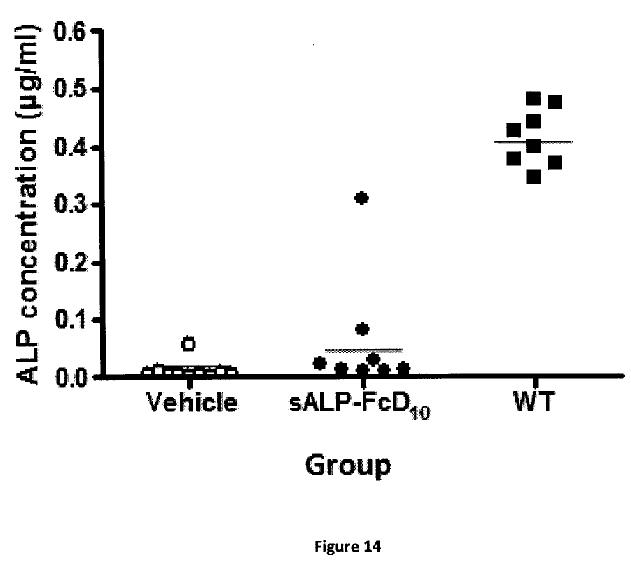

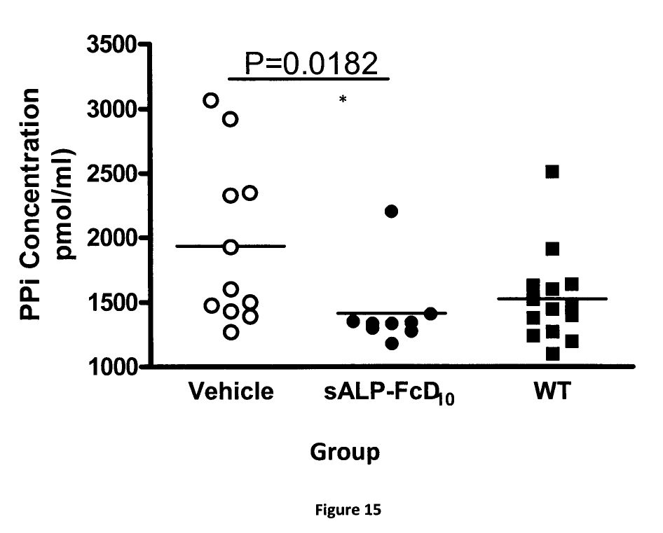

FIG. 14 shows that enzyme activities in serum at day 16 were barely above the detection level. Despite low serum values for sTNALP-FcD10, serum PPi levels were corrected (FIG. 15). Untreated Akp2^{-/-} mice had serum PPi concentrations of 1.90±0.64 μmol/ml, whereas treated Akp2^{-/-} mice had levels of 1.41±0.30 μmol/ml, comparable to those of WT mice (1.52±0.35 μmol/ml).

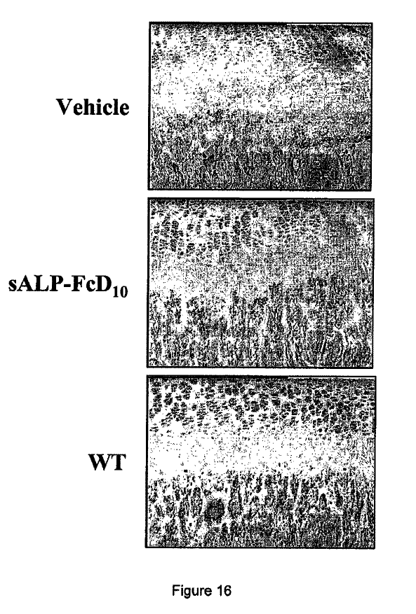

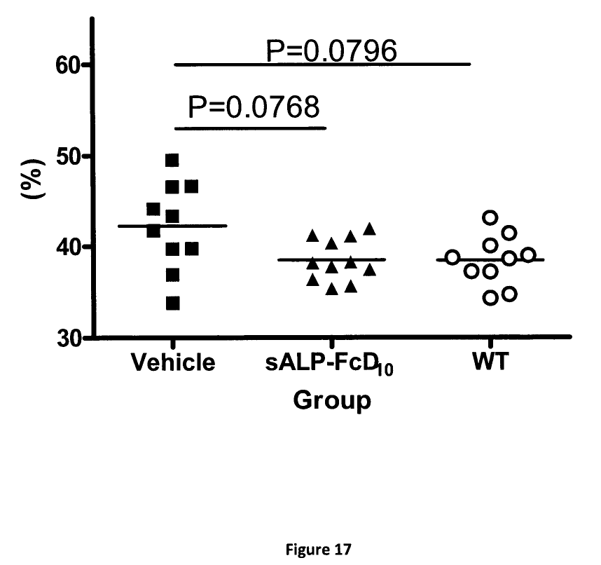

Proximal tibial growth plates showed some widening of the hypertrophic zone in Akp2^{-/-} animals compared WT animals (compare vehicle with wild-type in FIG. 16). The same observation made earlier in this strain of Akp2^{-/-} mice (Hessle et al. 2002) is consistent with rickets. A trend toward normalization of the physeal morphology was observed in animals treated with sTNALP-FcD10 for 15 days (FIG. 17) compared to vehicle (untreated).

### Example 10

### Short-Term (15 Days) Efficacy of High Doses (8.2 mg/kg) of sTNALP-FcD10 for HPP in Akp2^{-/-} Mice

To evaluate 15 days of daily s.c. injections using a significantly higher dose of sTNALP-FcD10 (8.2 mg/kg) on growth and bone mineralization, mice from 20 litters (141 mice total) were used. They were distributed to two groups: 1) Akp2^{-/-} mice given vehicle (N=19); 2) Akp2^{-/-} mice treated with sTNALP-FcD10 (N=20); additionally, there was one WT mouse per litter, non treated (N=18).

### Body Weight

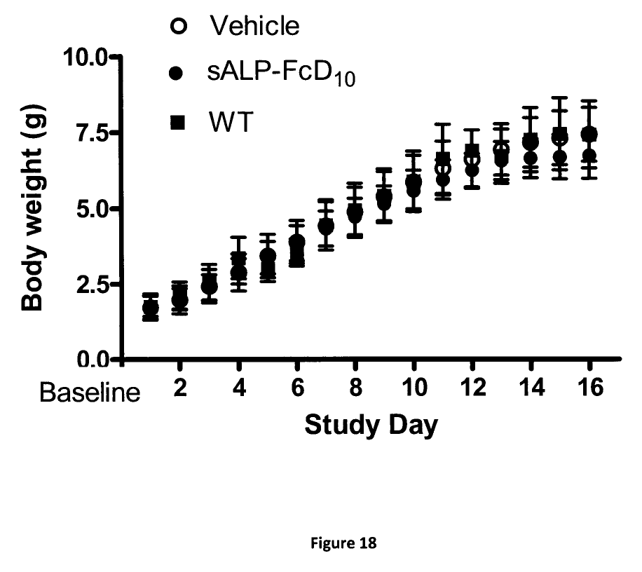

Akp2^{-/-} mice grew more slowly than WT mice. At day 1, no statistical difference in body weights was observed among the vehicle, sTNALP-FcD10, and WT animals. However, daily mean body weights diverged at day 6 (FIG. 18). The difference between WT (4.2±0.6 g) and vehicle (3.7±0.7 g) achieved statistical significance (p=0.0217) at day 6; but the difference between vehicle (5.9±1.0 g) and sTNALP-FcD10 treated values (6.7±1.0 g) achieved statistical significance at day 11 (p=0.04), with the treated group paradoxically heavier than WT. At day 16, mean body weight of treated animals (8.2±1.1 g) and WT (8.4±0.8 g) were not statistically different. Animals treated with sTNALP-FcD10 had body weights statistically greater (p=0.026) than those treated with vehicle (6.6±1.4 g). No significant difference between the ERT and WT groups was observed for body weight at any time point.

### Bone Length

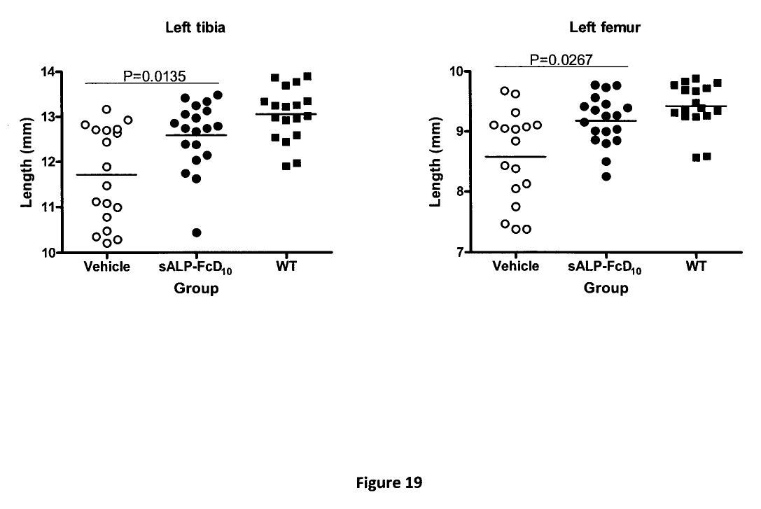

At the end of this experiment (day 16), tibial length provided an additional measure of skeletal benefit for Akp2^{-/-} mice. The tibia length with ERT was 12.6±0.7 mm and longer (p=0.0135) compared to animals given vehicle (11.7±1.1 mm) (FIG. 19). A statistical difference (p=0.0267) was also obtained when femur length was compared between the sTNALP-FcD10 (9.2±0.4 mm) and vehicle (8.6±0.8 mm) groups. No statistical difference was noted for tibia or femur length of the ERT compared to WT mice. A partial preservation (i.e. partial prevention of reduction in bone growth that becomes apparent around two weeks of age) of tibia and femur growth was observed by measures of length at necropsy (FIG. 19).

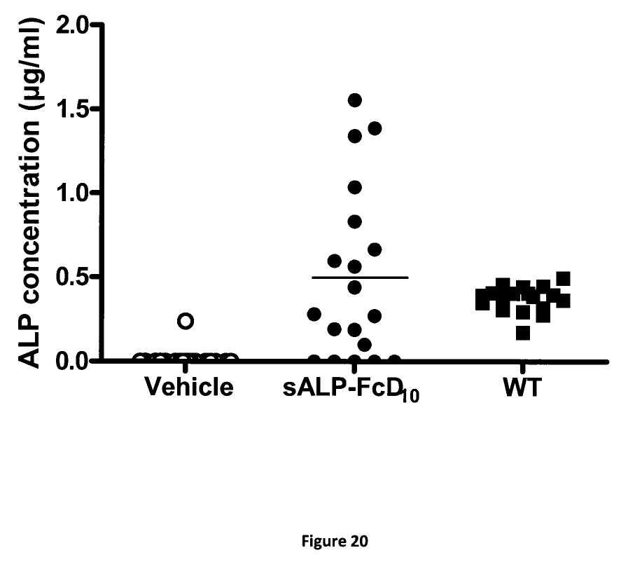

In all but 5 animals, detectable, but highly variable, levels of sTNALP-FcD10 were found in the plasma of treated Akp2^{-/-} mice at day 16 (FIG. 20). Circulating TNALP concentrations in normal animals are given for comparison purposes.

### Bone Mineralization

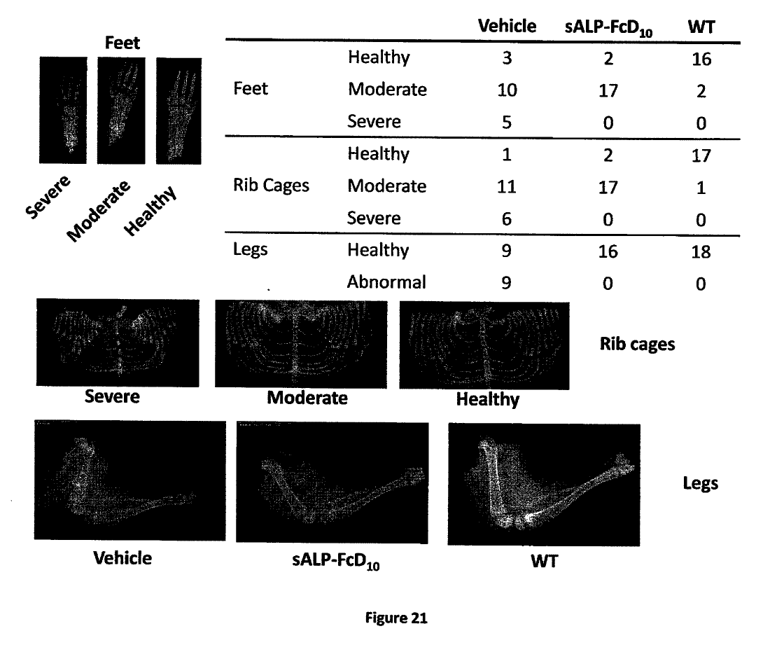

Blinded evaluations of Faxitron™ images of the feet and rib cages distinguished two degrees of severity of mineralization defects in the Akp2−/− mice (FIG. 21). Severely affected mice (Severe) had an absence of digital bones (phalanges) and secondary ossification centers. Moderately affected (Moderate) mice had abnormal secondary ossification centers, but all digital bones were present. WT mice (Healthy) had all bony structures present with normal architecture. Radiographic images of the hind limbs were similarly classified as abnormal if evidence of acute or chronic fractures was present, or healthy in the absence of any abnormal findings (FIG. 21). ERT minimized mineralization defects in the feet documented by the number of Akp2−/− mice with severe defects, consisting of 5 in the untreated group yet 0 in the ERT group (Table in FIG. 21). Chi-Square was significant (p≦0.05), indicating ERT decreased the severity of the acquired bone defects. Because severely affected infantile HPP patients often die from undermineralized and fractured ribs incapable of supporting respiration, the thoraces were also closely examined. ERT also reduced the incidence of severely dysmorphic rib cages (Table in FIG. 21). Chi-Square analysis was significant at p≦0.025. Similarly, the hind limbs appeared healthy in all treated animals (Table in FIG. 21). Chi-Square analysis was significant at p≦0.025.

### Dental Defects

Mandibles from 16-day-old mice were immersion-fixed overnight in sodium cacodylatebuffered aldehyde solution and cut into segments containing the first molar, the underlying incisor, and the surrounding alveolar bone. Samples were dehydrated through a graded ethanolseries and infiltrated with either acrylic (LR White) or epoxy (Epon 812) resin, followed by polymerization of the tissue-containing resin blocks at 55° C. for 2 days. Thin sections (1 μm) were cut on an ultramicrotome using a diamond knife, and glass slide-mounted sections were stained for mineral using 1% silver nitrate (von Kossa staining, black) and counterstained with 1% toluidine blue. Frontal sections through the mandibles (at the same level of the most mesial root of the first molar) provided longitudinally sectioned molar and cross-sectioned incisor for comparative histological analyses.

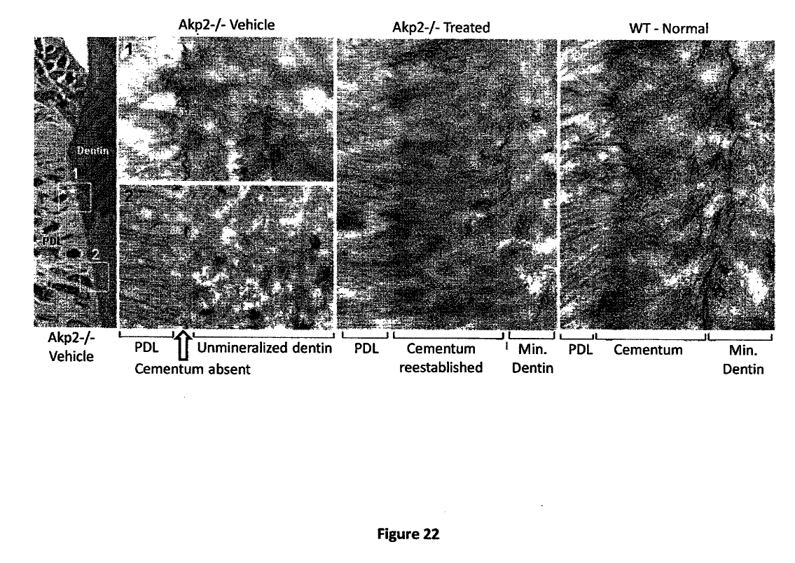

Histological examination of teeth from Akp2^{-/-} mice, shows poorly mineralized dentin tissue and very little cementum between the periodontal ligament and the dentin as compared to wild-type animals (FIG. 22, compare Akp2^{-/-} Vehicle and WT-Normal). Restored dentin mineralization and the formation of the cementum is also shown in FIG. 22 (Akp2^{-/-} Treated vs. WT-Normal).

### Example 11

### Long-Term (52 Days) Efficacy of High Doses (8.2 mg/kg) of sTNALP-FcD10 for HPP in Akp2^{-/-} Mice

Finally, to assess long-term survival and bone mineralization in Akp2^{-/-} mice, either sTNALP-FcD10 (8.2 mg/kg) or vehicle was given daily for 52 days (s.c. injections).

### Mice Survival, Activity and Appearance

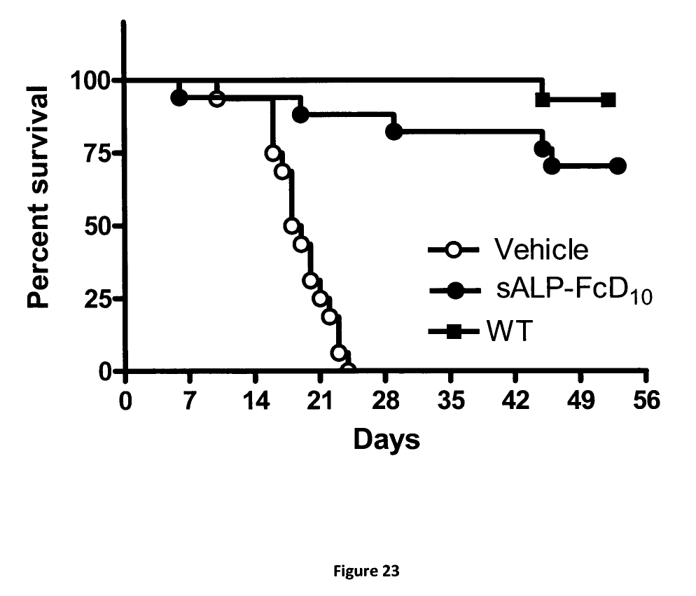

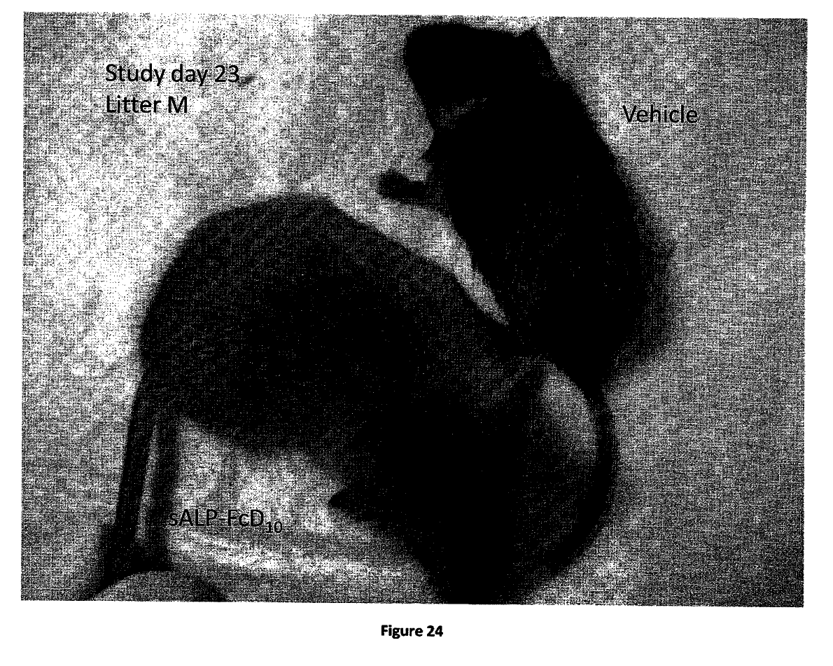

Untreated mice had a median survival of 18.5 days (FIG. 23) whereas survival was dramatically increased with ERT and this treatment also preserved the normal activity and healthy appearance (FIG. 24) of the treated mice.

### Bone Mineralization

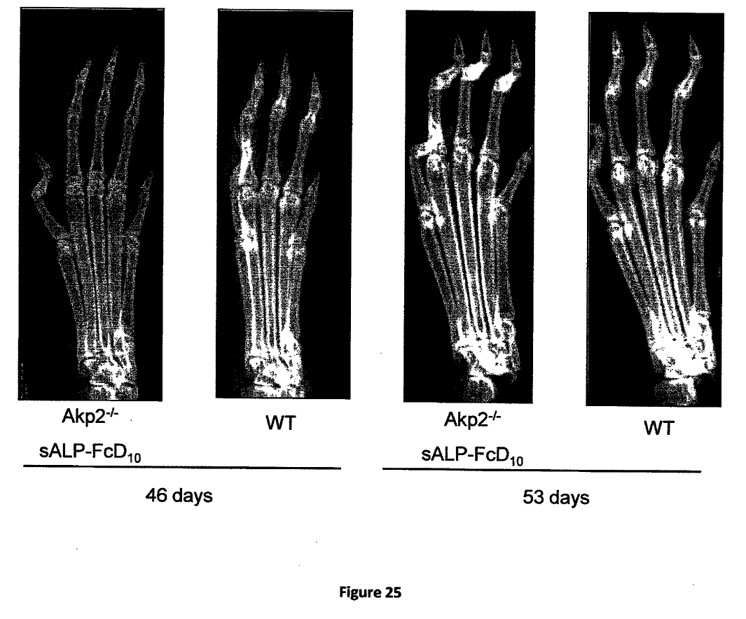

Radiographs of the feet of 16 day-old Akp2^{-/-} mice showed secondary ossification defects that are a hallmark of the disease (see FIG. 25). These defects were prevented in all treated mice by daily doses of sTNALP-FcD10 for 46 or 53 days (FIG. 25).

### ALP Activity

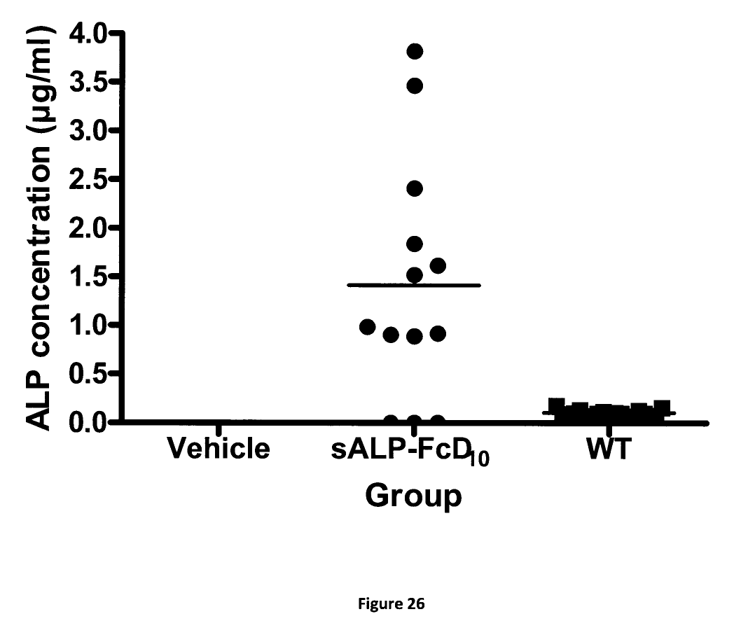

Plasma ALP activity levels were measured in treated Akp2^{-/-} mice after 53 days. FIG. 26 shows that most of the values were between 1 and 4 μg/ml of ALP activity. Three animals, however, had undetectable ALP levels.

Interestingly, unlike WT mice where a steady-state serum concentration of sTNALP-FcD10 was achievable, great variability in the serum levels of ALP was measured in the treated Akp2^{-/-} mice.

### Example 12

### Long-Term Efficacy of Different Dosage Intervals of sTNALP-FcD10 in Akp2^{-/-} Mice

Newborn Akp2^{-/-} mice were injected with 4.3 mg/kg daily (Tx-1), 15.2 mg/kg every 3 days (Tx-3) or 15.2 mg/kg every 7 days (Tx-7) of sTNALP-FcD10. Treatment was pursued for 43 days and mice were sacrificed on day 44, namely 24 hours after the last injection. They were monitored to evaluate any improvement of their survival and skeletal mineralization.

### Mice Survival

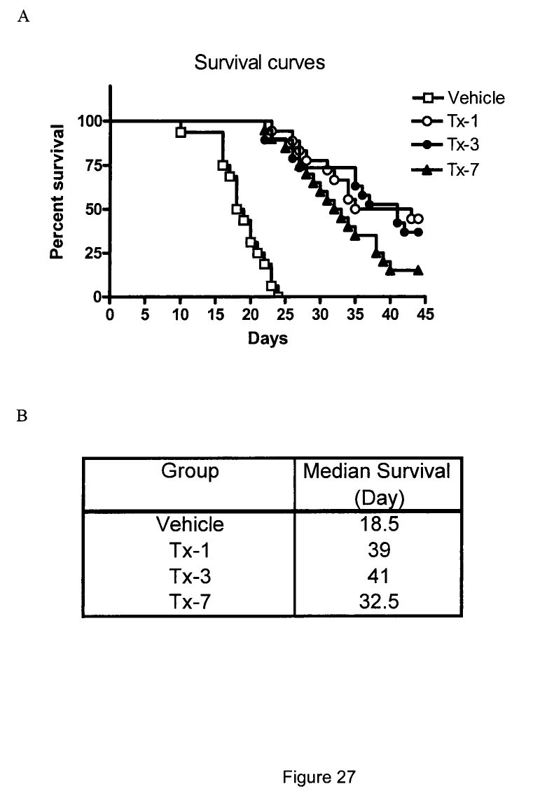

The survival of treated mice was increased compared to the mice that were injected vehicle (FIG. 27). This increase was statistically significant (p<0.0001). There was no statistically significant difference when the survival curves of treated groups were compared between themselves.

### Bone Mineralization

A) For each treatment, the radiographs of the feet were analyzed and distributed between normal and abnormal. Numbers and percentages (in parentheses) appear in Table 4 below. The bone mineralization defects were evaluated at day 23 and at the end of the study (Day 23-45).

TABLE 4
Distribution of radiographs of feet

| | | |
|---|---|---|
| Group | Abnormal (%) | Normal (%) |

Mid-Study (D23)

| | | | | | |
|---|---|---|---|---|---|
| Tx-1 | (N = 18) | 6 | (33) | 12 | (67) |
| Tx-3 | (N = 19) | 4 | (21) | 15 | (79) |
| Tx-7 | (N = 20) | 10 | (50) | 10 | (50) |
| WT | (N = 32) | 0 | (0) | 32 | (100) |

B)
D23-45

| | | | | | |
|---|---|---|---|---|---|
| Tx-1 | (N = 18) | 3 | (17) | 15 | (83) |
| Tx-3 | (N = 19) | 0 | (0) | 19 | (100) |
| Tx-7 | (N = 20) | 3 | (15) | 17 | (85) |
| WT | (N = 31) | 0 | (0) | 31 | (100) |

At mid study, sTNALP-FcD10 administered at 15.2 mg/kg every 3 days normalized bone mineralization defects in 79% of mice. This rate of normalization approached statistical significance when compared to the 50% rate of normalization evaluated in the mice treated with 15.2 mg/kg every 7 days (Chi Square; p=0.0596). No other inter treatment comparisons were statistically significant or approached significance.

At end of study, the percent of normalization improved in all treated groups compared to the percent normalization evaluated at day 23. The Chi Square test comparing the distribution among all sTNALP-FcD10 treatments was not significant (p=0.1844). The 100% rate of normalization observed in the mice treated with every 3 days approached statistical significance when compared to the rate in mice treated daily (83%, p=0.0634) or every 7 days (85%, p=0.0789).

However, in all treatment groups a significant proportion of the animals classified as abnormal at day 23 improved and became normal at end of the study. In the daily treatment group, 3 out of 6 animals normalized; in the mice treated every 3 days, 4 out of 4 improved, and finally in the weekly treatment group, 7 out of 10 became normal. Although dosage intervals provide satisfying results, the best results were obtained when the resulting daily amount administered was the highest.

### Example 13

### Long-Term Efficacy of High Doses (8.2 mg/kg) of sTNALP-FcD10 in 15 Day Old Akp2^{-/-} Mice

Efficacy studies as described in Example 11 were conducted in 15 day old mice which have started to manifest skeletal defects as observed on X-ray pictures of feet (see Example 11, FIG. 25). sTNALP-FcD10 was administered until the end of the study. The animals were monitored to evaluate any improvement of their survival, body weight and skeletal mineralization.

### Mice Survival

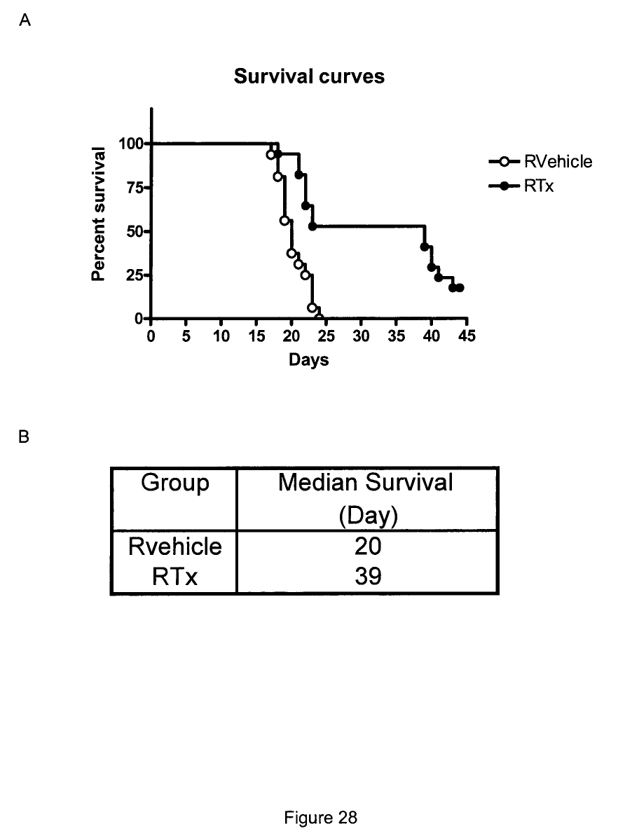

Daily injections, starting at day 15, of 8.2 mg/kg sTNALP-FcD10 to Akp2^{-/-} mice increased their survival compared to the mice that were injected vehicle (FIG. 28). This increase was statistically significant (p<0.05).

### Body Weight

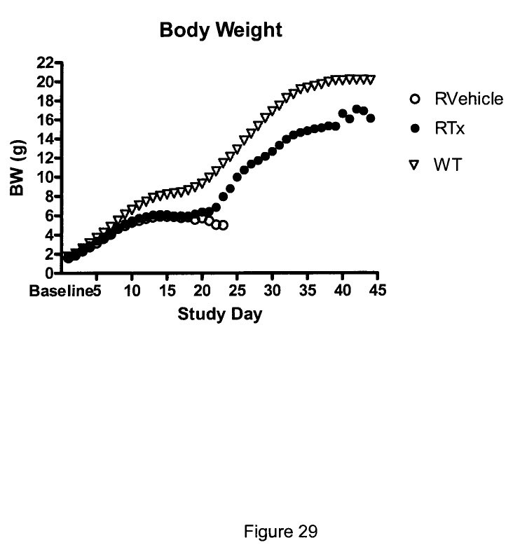

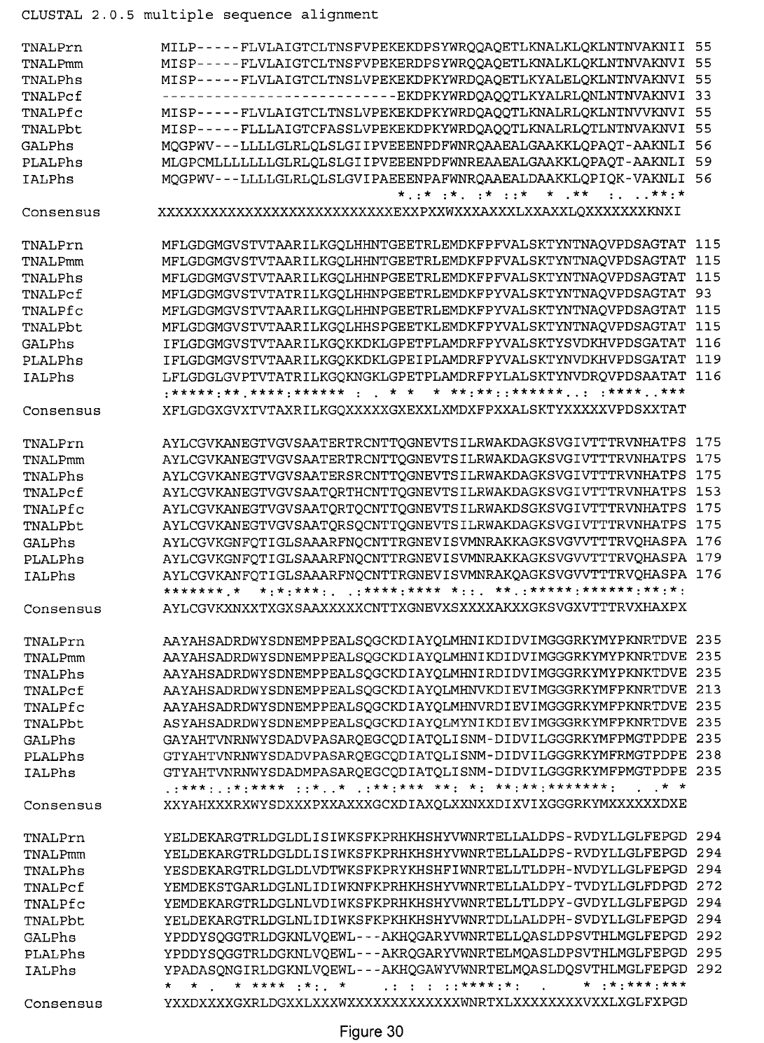

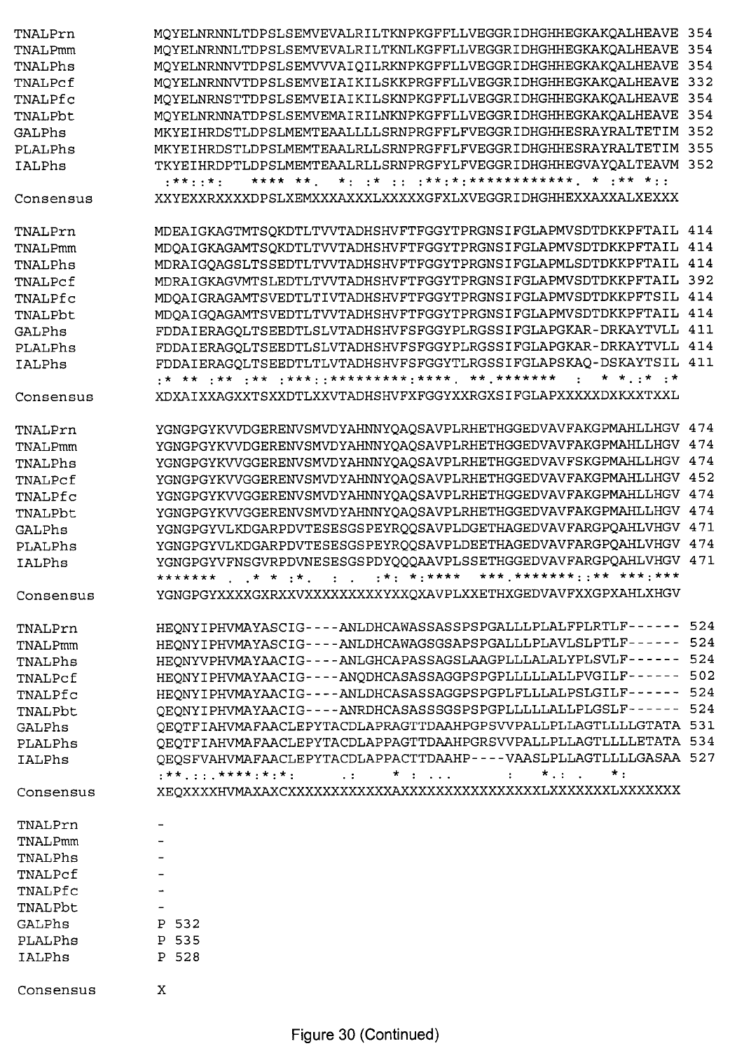

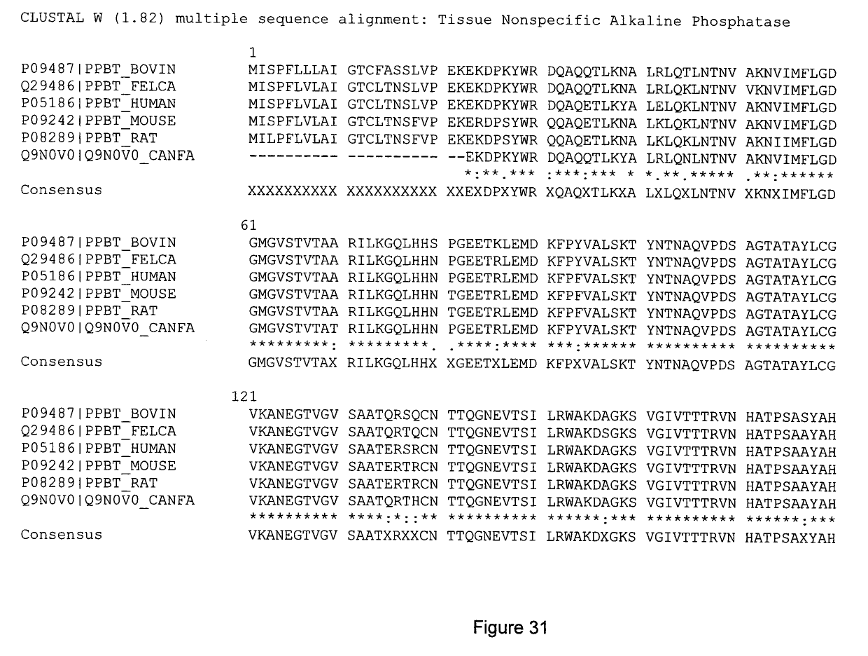

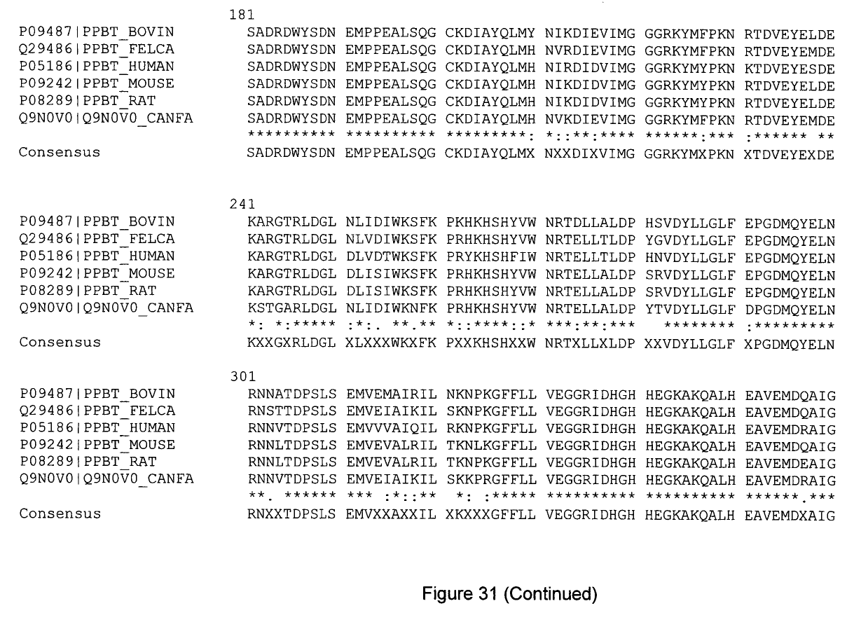

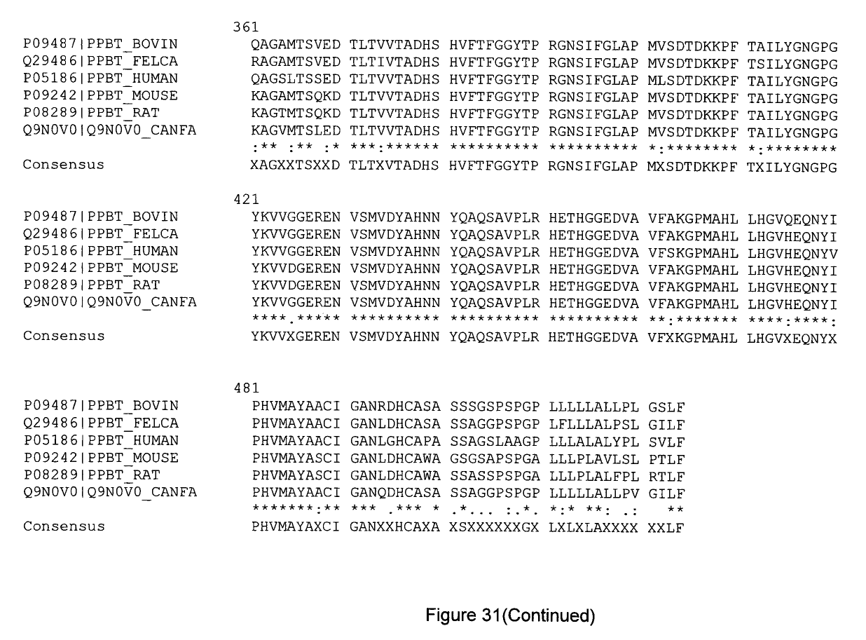

At the start of the study, no significant difference in body weight was noticed between groups (FIG. 29). At the beginning of treatment (day 15), the body weight of Akp2^{-/-} mice was smaller than that of wild-type animals. While the body weight of animals injected vehicle continued to decrease, Akp2^{-/-} mice treated with sTNALP-FcD10 started to gain weight 4 to 5 days after initiation of treatment and kept gaining weight until the end of the study, without however reaching the values of the wild-type animals. This weight gain suggests improvement in the well being of the animals treated with sTNALP-FcD10.

### Bone Mineralization

For each treatment, the radiographs of the feet were analyzed and distributed between normal and abnormal. Numbers and percentages (in parentheses) appear in Table 5. The radiographs were taken at necropsy.

Treatment of Akp2^{-/-} mice with 8.2 mg/kg sTNALP-FcD10 daily, starting at day 15 after birth improved mineralization as seen from the radiography of the feet taken at necropsy. The sTNALP-FcD10-treated group showed 41% normal animals compared to 12% in vehicle-injected group of Akp2^{-/-} mice. This difference almost reached statistical significance (p=0.0645 in Chi square test).

TABLE 5
Distribution of radiographs of feet

| Group | | Abnormal | Normal |
|---|---|---|---|
| RVehicle | (N = 16) | 14 (88) | 2 (12) |
| RTx-1 | (N = 17) | 10 (59) | 7 (41) |
| WT | (N = 30) | 0 (0) | 30 (100) |

### Example 14

### Long-Term Efficacy of Different Dosage Intervals of sTNALP-FcD10 on the Rescue of Akp2^{-/-} Mice

Mice were initiated on the treatment at day 12 and injected s.c. with vehicle (RV), 8.2 mg/Kg daily to days 46/47 (RTx-1) or injected with 8.2.mg/Kg daily for 7 days followed by 24.6 mg/Kg every 3 day (RTx-3) or followed by 57.4 mg/Kg every 7 days (RTx-7). The median survival was 19.5 days for the RV mice, 21.0 days for the RTx-7 mice, 30.5 days for the RTx-3 mice and 37.5 days for the RTx-1 mice. In all cases, survival was statistically increased when compared to that of the vehicle-treated group. There is a clear benefit of ERT in Akp2^{-/-} mice with well-established hypophosphatasia. Dosing less frequently than daily also appears to statistically increase survival.

### Example 15

### A Maximum Tolerated Dose Intravenous Injection Toxicity Study in Juvenile Sprague-Dawley Rats

The objective of the study was to determine the maximum tolerated dose (MTD) and toxicity of the test article, sTNALP-FcD10, following repeated administration to juvenile Sprague-Dawley rats by intravenous injection. In Examples 15 to 18, the sALP-FcD10 used is that specifically described in FIG. 3.

sTNALP-FcD10 was administered to juvenile Sprague-Dawley rats (aged at initiation between 22 and 24 days) once weekly for four weeks by intravenous injection as described in Table 6 below:

TABLE 6
Study design

| | | Dose Level | Dose | Number of | |
|---|---|---|---|---|---|
| Group | | (mg/kg/ | Concentration | Animals | |
| Numbers | Treatment | occasion) | (mg/mL) | Male | Female |
| 1 | Dose 1 | 10 | 2.0 | 3 | 3 |
| 2 | Dose 2 | 30 | 6.0 | 3 | 3 |
| 3 | Dose 3 | 90 | 18.0 | 3 | 3 |
| 4 | Dose 4 | 180 | 36.0 | 3 | 3 |

Throughout the study, the animals were monitored for mortality, body weight, and clinical condition. Hematology, coagulation and clinical chemistry assessments were performed on all animals. Terminally, the rats were euthanized and subjected to necropsy. For each animal, samples of selected tissues were retained and were subjected to histological processing and microscopic examination.

There was no mortality in this study and there were no test article-related changes in coagulation parameters or organ weights. The body weights of the High Dose males, in particular, were about 10% below the Low Dose suggesting a treatment-related effect.

No clinical signs were observed in Groups 1 and 2 animals on the first dosing occasion. In Groups 3 and 4 animals, however, the animals appeared weak immediately following dosing and some Group 4 animals showed slight to moderate decrease in activity. Slight swelling of limbs, pinna and muzzle with skin discoloration (red or blue in appearance) at the extremities were also observed in the two groups. Other clinical signs observed in Group 4 animals included excessive scratching, piloerection and hyperpnea.

Clinical signs recorded for Groups 1 and 2 animals on the second dosing occasion (Day 8), were swelling of limbs, pinna and muzzle with skin discoloration (red or blue in appearance) at the extremities. Similar clinical signs of skin swelling were also recorded on the third and fourth dosing occasions (Days 15 and 22) for the same groups of animals. On the fourth dosing occasion (Day 22) slight hyperactivity was observed in Group 1 females whereas hypoactivity was observed in Group 2 males. For Group 3 and 4 animals, the clinical signs of reduced motor activity, piloerection, hyperpnea and swelling of limbs, pinna and muzzle with skin coloration became more evident as dosing progressed from the first dosing occasion to the fourth. It is considered that these clinical signs were treatment-related. On Days 16 to 19 and on Day 23, slight swelling and skin coloration of pinna (red in appearance) were observed in one animal (Group 1). Similar clinical signs were observed in another animal (Group 2) on Day 23.

The clinical signs were acute and the severity increased as dosing progressed but they were transient. All clinical signs appeared within 50 minutes after administration of test article, sTNALP-FcD10, with some animals recovering within, approximately, thirty minutes to 2 hours. For other animals, recovery was complete the next morning (the next scheduled observation time).

There was a treatment-related decrease in platelet counts (PLT) for males and females from all treatment groups, measured after the last dose, compared to background values. There was an increase in predominantly the percentage but also absolute reticulocytes that was noted generally in animals treated at the three highest dose levels.

Levels of alkaline phosphatase in serum were higher than could be quantified by the analytical instrument even following dilution. The results that were available for the Low Dose females were dramatically higher than the background range. This was expected as the test article is an active modified ALP.

Macroscopically, dark focus/area and/or depressed area of the glandular stomach were observed in 3 of 6 Group 3 animals (2 males/1 female) and 4 of 6 Group 4 animals (2 males/2 females).

Microscopically, minimal to mild erosion/ulcer of the glandular stomach, occasionally associated with submucosal edema was noted in 3 of 6 Group 3 animals (2 males/1 females) and 4 of 6 Group 4 animals (2 males/2 females), correlating gross findings.

In conclusion, intravenous injection of sTNALP-FcD10 to juvenile Sprague-Dawley rats once weekly for 4 weeks did not cause death at any of the dose levels tested but did cause adverse clinical signs, minor haematological changes and erosion/ulceration of the glandular stomach, occasionally associated with submucosal edema at dose levels of 90 and 180 mg/kg.

Changes related to administration of the test article at the two lowest dose levels tested (10 and 30 mg/kg) were limited to transient clinical signs apparent on the day of dosing only. The clinical signs were more severe at the 90 mg/kg dose level but they were also transient. The clinical signs noted in the animals treated with 180 mg/kg were so severe as to prevent this dose level from being used in future studies. Consequently the highest recommended dose level for subsequent longer term studies is 90 mg/kg.

### Example 16

### An Intravenous Injection and Infusion Maximum Tolerated Dose Toxicity Study in Juvenile Cynomolgus Monkeys

The purpose of this study was to determine the maximum tolerated dose for sTNALP-FcD10, when administered once by intravenous injection or infusion to juvenile Cynomolgus monkeys. The test article dosing formulations were administered once in an incremental fashion, as indicated in Table 7 below.

TABLE 7
Study design

| | | | Dose | Dose | Dose | Number of Animals | | | |
|---|---|---|---|---|---|---|---|---|---|
| Study | | Dose Level | Volume | Rate | Conc. | Main Study | | Toxicokinetic | |
| Day | Treatment | (mg/kg) | (mL/kg) | (mL/kg/hr) | (mg/mL) | Males | Females | Males | Females |
| 1 | IV Injection | 5 | 4 | N/A | 1.25 | 2 | 2 | 1 | 1 |
| 8 | IV Injection | 15 | 4 | N/A | 3.75 | | | | |
| 15 | IV Infusion | 45 | 4 | 80 | 11.25 | | | | |
| 22 | IV Infusion | 90 | 4 | 40 | 22.5 | | | | |
| 29 | IV Infusion | 180 | 4 | 20 | 45 | | | | |
| 46* | IV Injection | 45 | 4 | N/A | 11.25 | | | | |

*Only the Main Study animals were dosed on Day 46.

After the last treatment, the animals were released from the study. Parameters monitored during the study were mortality, clinical observations, body weights, appetence, toxicokinetics, hematology and clinical chemistry.

No mortality, adverse clinical signs or effect on body weights were observed during the study.

A marked dose proportional increase in alkaline phosphatase was observed in all animals throughout the study. Since the test article was a synthetic alkaline phosphatase, this increase was principally due to the presence of the drug in the bloodstream of the animals after each dosing.

Increases in alanine aminotransferase and aspartate aminotransferase were observed in three animals during the study but in the absence of a necropsy, the toxicological significance of this finding is uncertain.

The pharmacokinetic of sTNALP-FcD10 was well characterized following a single IV administration of 5, 15, 45, 90 and 180 mg/kg to monkeys. For the IV injections mean AUC∞ values ranged from 797 to 2950 mg·h/L and mean Cmaxvalues ranged from 65 to 396 mg/L over the dose range studied. For the infusions, mean AUC∞ ranged from 9410 to 48400 mg·h/L and Cmaxranged from 1230 to 7720 mg/L over the dose range studied.

Mean t1/2 values of sTNALP-FcD10 appeared to decrease with increasing dose levels of sTNALP-FcD10. Although systemic clearance of sTNALP-FcD10 was relatively consistent across dose levels, the 90 mg/kg dose group appeared to be a pharmacokinetic outlier with a substantially lower clearance when compared to the other dose levels (approximately five fold). No obvious gender related trends were noted.

In summary, although some reversible blood chemistry changes were observed during the study, the intravenous injection/infusion of sTNALP-FcD10 at up to 180 mg/kg was well tolerated by the juvenile Cynomolgus monkeys. Therefore, under the conditions of this study, the Maximum Tolerated Dose was considered to be at least 180 mg/kg.

### Example 17

### A 4-Week (Once Weekly) Intravenous Injection Toxicity Study of sTNALP-FcD10 in the Juvenile Albino Rat Followed by a 28-Day Recovery Period

The objective of this study was to investigate the potential toxicity of sTNALP-FcD10 given once weekly by intravenous injection to the juvenile rat for a minimum of 4 consecutive weeks (total of 4 doses) followed by 28 days of recovery. The animals were dosed on study days 1, 8, 15 and 22 and the recovery period began on study day 29. The study design is detailed in Table 8 below.

TABLE 8
Study design

| | Target Dose | Actual Dose | Target | Actual | | | | |
|---|---|---|---|---|---|---|---|---|
| | Level | Level | Concentration | Concentration | Main Study | | Recovery Study | |
| Groups | (mg/kg/dose) | (mg/kg/dose) | (mg/mL) | (mg/mL) | Males | Females | Males | Females |
| 1 - | 0 | 0 | 0 | 0 | 10 | 10 | 5 | 5 |
| Vehicle | | | | | | | | |
| Control | | | | | | | | |
| 2 - Low | 3 | 2.5 | 0.6 | 0.5 | 10 | 10 | 5 | 5 |
| Dose | | | | | | | | |
| 3 - Mid | 30 | 26 | 6 | 5.1 | 10 | 10 | 5 | 5 |
| Dose | | | | | | | | |
| 4 - High | 90 | 77 | 18 | 15.3 | 10 | 10 | 5 | 5 |
| Dose | | | | | | | | |

The following were evaluated: clinical signs (twice daily), body weight (once during acclimation period and weekly starting on Day 21 post partum), food consumption (weekly), opthalmology (end of treatment and end of recovery period), hematology (at necropsy), serum chemistry (at necropsy), urinalysis (Day 29 and at the end of recovery period), biochemical markers of bone turnover: osteocalcin (bone formation marker) and C-telopeptide (bone resorption marker) (the morning prior to schedule necropsy), antibody assessment (Day −1 and at necropsy), test article blood concentration evaluation (Day 16 and Day 23), bone densitometry (by DXA in vivo Day −1, 28-main and recovery study animals and Day 14 and 56-recovery study animals and pQCT ex vivo), radiography and macroscopic observations at necropsy, organ weights and histopathology.

One male given 90 mg/kg/dose was found dead on study Day 25. As no consequential histological observations (pulmonary and thymic hemorrhages graded slight and minimal, respectively) were made for this rat, its cause of death was undetermined based on pathological investigations. On Day 23, this animal was bled for test article blood concentration evaluation and this procedure may have contributed to its death since there was no evidence of toxicity on Day 22. There were no sTNALP-FcD10-related mortality or effects on opthalmology, urinalysis, bone formation marker (osteocalcin), organ weights, gross pathology, radiology or microscopy examination.

sTNALP-FcD10-related clinical signs observed at 3, 30 and/or 90 mg/kg/dose groups are considered to be acute infusion reaction. These included partly closed eyes, decreased muscle tone, lying on the side, hunched posture, cold to touch, uncoordinated movements, decreased activity, abnormal gait and/or blue, red and/or firm swollen hindpaws and/or forepaws during cage-side observations at 5, 15, 30 and/or 60 minutes post dose. These observations were transient and did not occur on nondosing days or during the recovery period.

Generally, a trend for slightly decreased body weight and body weight gain was noted for males in the 3, 30 and/or 90 mg/kg/dose groups during the recovery period. The effect on bone size on two bones of appendicular skeleton (femur and at tibia) correlated with decreased body weights. Decreases in food consumption were generally consistent with the decreased body weights. Body weights were comparable to controls for sTNALP-FcD10-treated females.

sTNALP-FcD10 administered at 90 mg/kg/dose was generally associated with slight decreases in absolute neutrophils, monocytes and/or eosinophils compared to the control group. Additionally, slight increases in lymphocytes, platelets and absolute reticulocytes were observed compared to the control group. At the end of the recovery period, these slight changes were still apparent in the animals treated with 90 mg/kg.

sTNALP-FcD10 was generally associated with statistically significant dose-related increases in alkaline phosphatase in all treated groups compared to controls. Considering the nature of the test article (alkaline phosphatase), the absence of any changes in other liver enzymes and absence of histopathological correlates, these increases are likely attributed to circulating levels of sTNALP-FcD10. Slight statistically significant increases in phosphorus were observed in males treated with sTNALP-FcD10 at 90 mg/kg/dose during Week 4, associated with a non-significant increases in serum total calcium. At the end of the recovery, these changes, including those statistically significant, returned to control values.

There were no organ weight, radiological, macroscopic or microscopic changes that were related to sTNALP-FcD10 in juvenile rats treated intravenously once weekly at up to 90 mg/kg/dose for 4 consecutive weeks. There were no delayed effects identified in a subset of these animals allowed a 28-day recovery after completion of the treatment.

Slightly lower mean CTx values were observed for treated females compared to controls (attaining statistical significance at 90 mg/kg/dose). These lower values were not consistent with the bone density analysis and also with the results obtained for males, therefore the incidental nature for these decreases cannot be excluded.

High variability in bone densitometry and bone geometry parameters noted between groups was attributed to the rapid growth phase. At the end of recovery, area and BMC (assessed by DXA and pQCT) were generally lower for treated males, suggesting smaller bones for these animals. The effect on bone size was noted on two bones of appendicular skeleton (femur and at tibia) by two different techniques, however no consistent effect was noted for axial skeleton (suggesting no effect on crown to rump length). Although area and BMC were decreased, the mean BMD values were generally comparable to controls, suggesting the effect on BMC and area was secondary to the effect on growth. Lower body weights and lower food consumption for treated males relative to controls are consistent with these data. However small group size at recovery, lack of consistency with respect to gender as well as the variability confounded these results, therefore an incidental nature for these decreases cannot be completely excluded.

In conclusion, once weekly intravenous injection to the juvenile rat for a minimum of 4 consecutive weeks followed by 28-day of recovery at doses of 3, 30 and 90 mg/kg/dose resulted in clinical signs associated with transient injection related effects including uncoordinated and reduced activity and paw swelling observed up to 60 minutes post-dose. Males treated at 90 mg/kg/dose showed slight decreases in body weight and food consumption which correlated with slightly smaller tibiae and femurs assessed by densitometry techniques. For females, slightly lower mean values were obtained for C-telopeptide levels compared to controls. Serum phosphorus levels were slightly, although significantly, increased in the 90 mg/kg/dose group. Elevated serum alkaline phosphatase levels were likely attributed to circulating levels of sTNALP-FcD10. sTNALP-FcD10 had no meaningful or consistent effects on bone densitometry and bone geometry for females during treatment and recovery period. For males no biologically significant effects were noted on bone densitometry or bone geometry during the treatment period. In general, slight decreases in bone densitometry (bone mineral content and/or area assessed by DXA and pQCT) and bone geometry parameters with a corresponding lower mean body weight were noted for males relative to controls at the end of the recovery period. All findings resolved after a 28-day treatment-free period with the exception of the effects on body weight and bone size for high dose males which persisted. There were no evidence of ectopic calcification at the end of treatment or the end of the recovery period. There were no radiological, macroscopic or microscopic findings as well as any organ weight changes associated with sTNALP-FcD10 treatment at any dose level. Because the injection reaction was transient and did not result in any effect on any parameters used to assess toxicity in the 3 and 30 mg/kg/dose groups, it was not considered to be adverse. In the 90 mg/kg/dose group, this reaction was more severe and accompanied by decreases in body weight gain, reduced food consumption, and potentially decrease in bone growth and therefore the effects in this group were considered to be adverse. Consequently, the no observable adverse effect level (NOAEL) was considered to be 30 mg/kg/dose in this study.

### Example 18

### A 4-Week Intravenous Injection Toxicity Study in Juvenile Cynomolgus Monkeys Followed by a 28-Day Recovery Period

The purpose of this study was to determine the toxicity and toxicokinetics of sTNALP-FcD10 in juvenile Cynomolgus monkeys, when administered once weekly by slow bolus intravenous injection for 4 weeks and to assess reversibility of any changes following a 28-day recovery period.

The control and test article dosing formulations were administered to juvenile Cynomolgus monkeys by slow intravenous bolus injection once weekly for 4 weeks followed by a 28-day recovery period, as indicated in the Table 9 below:

TABLE 9
Study design

| | | | | Number of Animals | | | |
|---|---|---|---|---|---|---|---|
| | Dose Level | Dose Volume | Dose Conc. | Main Study | | Recovery | |
| Group | (mg/kg) | (mL/kg) | (mg/mL) | Males | Females | Males | Females |
| 1 Control* | 0 | 4 | 0 | 3 | 3 | 2 | 2 |
| 2 Low Dose | 5 | 4 | 1.25 | 3 | 3 | 2 | 2 |
| 3 Mid Dose | 15 | 4 | 3.75 | 3 | 3 | 2 | 2 |
| 4 High Dose | 45 | 4 | 11.25 | 3 | 3 | 2 | 2 |

*The Group 1 animals received the vehicle/control article, 25 mM sodium phosphate pH 7.4, 150 mM NaCl.

After the last treatment (Day 22), the Main Study animals were euthanized on Day 29, while the remaining Recovery animals were observed for an additional 28 days, following which they were euthanized on Day 57. All Main and Recovery animals were subjected to a necropsy examination.

Evaluations conducted during the study or at its conclusion included mortality, clinical condition, body weight, appetence, body measurements, radiographic assessments of bone development, opthalmology, electrocardiography, toxicokinetics, immunogenicity, hematology, coagulation, clinical chemistry, urinalysis, biomarkers of bone turnover, organ weights, ex-vivo bone mineral density analyses, and gross and histopathology.

No mortality or adverse treatment-related clinical observations were noted during the study.

Based on the body measurements recorded at the end of the treatment and recovery period, there were no noteworthy inter-group differences for cranial circumference, or humerus, forearm, tibia or pelvic limb lengths.

There were no body weight or food consumption changes related to treatment with the test article at any dose level. There were no opthalmological or electrocardographic findings related to the test article at any dose level. There were no haematological, red cell morphological, coagulation or urinalysis changes related to treatment with the test article at any dose level. There were no toxicologically significant changes among clinical biochemistry parameters during the treatment or recovery periods. A slight to pronounced dose related increase in alkaline phosphatase was observed in all test article treated animals at most assessment occasions throughout the treatment period. Alkaline phosphatase levels were generally more comparable to control values by the end of the recovery period. Since the test article is a synthetic alkaline phosphatase, this increase was principally due to the presence of the drug in the bloodstream of the animals after each dose, and thus the increases were considered to be non-adverse.

At the end of the treatment and recovery periods, there were no noteworthy inter-group differences in absolute or relative organ weights, nor were there any test article-related macroscopic or microscopic findings. Histological changes noted were considered to be either incidental findings, common background findings in this species, or findings related to some aspect of experimental manipulation. Reproductive organs were generally immature but considered normal for this age monkey.

In conclusion, weekly intravenous injection of sTNALP-FcD10, to male and female Cynomolgus monkeys for 4 weeks, at dose levels of 0, 5, 15 and 45 mg/kg, and followed by a 4-week recovery period, was without evidence of toxicity at any dose level. Therefore the high dose level, 45 mg/kg, was considered to be the No Observed Adverse Effect Level (NOAEL) in this study.

### Example 19

### Determination of Maximum Recommended Starting Dose for Human

The maximum recommended starting dose (MRSD) for human is calculated by establishing the No Observed Adverse Effect Level (NOAEL, see Guidance for Industry and Reviewers. December 2002). Various concentrations of the formulation described above have been tested on mice, rat and monkeys including 1 mg/kg, 5 mg/kg, and 8.2 mg/kg daily subcutaneously; 3 mg/kg, 5 mg/kg, 10 mg/kg, 30 mg/kg, 45 mg/kg, 90 mg/kg and 180 mg/kg. The NOAEL for the most sensitive species, namely for rat, was 30 mg/kg.

This dose was scaled up to a human equivalent dose (HED) using published conversion tables which provide a conversion factor from rat to human of 6. A NOAEL of 30 mg/kg for that species is equivalent to 5 mg/kg in human.

This value (5 mg/kg) was divided by a security factor of ten. The calculated MRSD is thus 0.5 mg/kg. For an average human weighting 60 kg, a weekly dose of 30 mg or daily dose of 4.28 mg daily could thus be injected to start clinical trials.

Although the present invention has been described hereinabove by way of specific embodiments thereof, it can be modified, without departing from the spirit and nature of the subject invention as defined in the appended claims.

### REFERENCES

1. Ali, S. Y., Sajdera, S. W. & Anderson, N. C. Isolation and characterization of calcifying matrix vesicles from epiphyseal cartilage. Proc Natl Acad Sci U S A 67, 1513-20 (1970).
2. Anderson, H C, Hsu, H. H., Morris, D. C., Fedde, K. N. & Whyte, M. P. 1997 Matrix vesicles in osteomalacic hypophosphatasia bone contain apatite-like mineral crystals. Am J Pathol 151 1555-61.
3. Anderson H C, Garimella R & Tague S E 2005a The role of matrix vesicles in growth plate development and biomineralization. Frontiers in Bioscience 10 822-837.
4. Anderson H C, Harmey D, Camacho N P, Garimella R, Sipe J B, Tague S, Bi X H, Johnson K, Terkeltaub R & Millan J L 2005b Sustained osteomalacia of long bones despite major improvement in other hypophosphatasia-related mineral deficits in tissue nonspecific alkaline phosphatase/nucleoticle pyrophosphatase phosphodiesterase 1 double-deficient mice. American Journal of Pathology 166 1711-1720.
5. Anderson H C & Reynolds J J 1973 Pyrophosphate stimulation of calcium uptake into cultured embryonic bones. Fine structure of matrix vesicles and their role in calcification. Developmental Biology 34 211-227.
6. Anderson H C, Sipe J B, Hessle L, Dhamyamraju R, Atti E, Camacho N P & Milan J L 2004 Impaired Calcification Around Matrix Vesicles of Growth Plate and Bone in Alkaline Phosphatase-Deficient Mice. American Journal of Pathology 164 841-847.
7. Bernard, G. W. 1978Ultrastructural localization of alkaline phosphatase in initial intramembranous osteogenesis. Clin Orthop, 218-25.
8. Di Mauro S, Manes T, Hessle L, Kozlenkov A, Pizauro J M, Hoylaerts M F & Millan J L 2002 Kinetic characterization of hypophosphatasia mutations with physiological substrates. Journal of Bone and Mineral Research 17 1383-1391.
9. Farley J R & Magnusson P 2005 Effects of Tunicamycin, Mannosamine, and Other Inhibitors of Glycoprotein Processing on Skeletal Alkaline Phosphatase in Human Osteoblast-Like Cells. Calcified Tissue International 76 63-74.
10. Fedde K N, Blair L, Silverstein J, Coburn S P, Ryan L M, Weinstein R S, Waymire K, Narisawa S, Milian J L, MacGregor G R & Whyte M P 1999 Alkaline phosphatase knock-out mice recapitulate the metabolic and skeletal defects of infantile hypophosphatasia. Journal of Bone and Mineral Research 14 2015-2026.
11. Greenberg, C. R. et al. 1993 A homoallelic Gly317—>Asp mutation in ALPL causes the perinatal (lethal) form of hypophosphatasia in Canadian mennonites. Genomics 17, 215-7.
12. Harmey D, Johnson K A, Zelken J, Camacho N P, Hoylaerts M F, Noda M, Terkeltaub R & Milian J L 2006 Elevated skeletal osteopontin levels contribute to the hypophosphatasia phenotype in Akp2(−/−) mice. Journal of Bone and Mineral Research 21 1377-1386.
13. Harmey D, Hessle L, Narisawa S, Johnson K A, Terkeltaub R & Millan J L 2004 Concerted Regulation of Inorganic Pyrophosphate and Osteopontin by Akp2, Enpp1, and Ank: An Integrated Model of the Pathogenesis of Mineralization Disorders. American Journal of Pathology 164 1199-1209.
14. Hawrylak K & Stinson R A 1988 The solubilization of tetrameric alkaline phosphatase from human liver and its conversion into various forms by phosphatidylinositol phospholipase C or proteolysis. Journal of Biological Chemistry 263 14368-14373.
15. Henthorn, P. S., Raducha, M., Fedde, K. N., Lafferty, M. A. & Whyte, M. P. 1992a Different missense mutations at the tissue-nonspecific alkaline phosphatase gene locus in autosomal recessively inherited forms of mild and severe hypophosphatasia. Proc Natl Acad Sci USA 89, 9924-8.
16. Henthorn, P. S. & Whyte, M. P. 1992b Missense mutations of the tissue-nonspecific alkaline phosphatase gene in hypophosphatasia. Clin Chem 38, 2501-5.
17. Hessle L, Johnson K A, Anderson H C, Narisawa S, Sali A, Goding J W, Terkeltaub R & Millan J L 2002 Tissue-nonspecific alkaline phosphatase and plasma cell membrane glycoprotein-1 are central antagonistic regulators of bone mineralization. Proceedings of the National Academy of Sciences of the United States of America 99 9445-9449.
18. Ikezawa H 2002 Glycosylphosphatidylinositol (GPI)-Anchored Proteins. Biol Pharm. Bull. 25(4) 409-417.
19. Jansonius J N 1998 Structure, evolution and action of vitamin B6-dependent enzymes. Current Opinion in Structural Biology 8 759-769.
20. Johnson K, Moffa A, Chen Y, Pritzker K, Goding J & Terkeltaub R 1999 Matrix vesicle plasma cell membrane glycoprotein-1 regulates mineralization by murine osteoblastic MC3T3 cells. Journal of Bone and Mineral Research 14 883-892.
21. Mahmood I, Green M D, & Fisher J E 2003 Selection of the First-Time Dose in Humans: Comparison of Different Approaches Based on Interspecies Scaling of Clearance. J. Clin. Pharmacol., 43 (7), 692-7.
22. Meyer, J. L. 1984 Can biological calcification occur in the presence of pyrophosphate? Arch Biochem Biophys 231 1-8.
23. Milián, J. L. 2006 Mammalian Alkaline Phosphatases. From Biology to Applications in Medicine and Biotechnology, Wiley-VCH Verlag GmbH & Co., Weinheim, Germany 1-322.
24. Morris, D. C., Masuhara, K., Takaoka, K., Ono, K. & Anderson, H. C. 1992 Immunolocalization of alkaline phosphatase in osteoblasts and matrix vesicles of human fetal bone. Bone Miner 19 287-98.
25. Murshed M, Harmey D, Millan J L, McKee M D & Karsenty G 2005 Unique coexpression in osteoblasts of broadly expressed genes accounts for the spatial restriction of ECM mineralization to bone. Genes and Development 19 1093-1104.
26. Nasu M, Ito M, Ishida Y, Numa N, Komaru K, Nomura S and Oda K 2006 Aberrant interchain disulfide bridge of tissue-nonspecific alkalinephosphatase with an Arg433. Cys substitution associated with severe hypophosphatasia FEBS Journal 273 5612-5624.
27. Narisawa S, Frohlander N & Millan J L 1997 Inactivation of two mouse alkaline phosphatase genes and establishment of a model of infantile hypophosphatasia. Developmental Dynamics 208 432-446.
28. Narisawa S, Wennberg C & Millan J L 2001 Abnormal vitamin B6 metabolism in alkaline phosphatase knock-out mice causes multiple abnormalities, but not the impaired bone mineralization. Journal of Pathology 193 125-133.
29. Nishioka T, Tomatsu S, Gutierrez M A, Miyamoto K, Trandafirescu G G, Lopez P L C, Grubb J H, Kanai R, Kobayashi H, Yamaguchi S, Gottesman G S, Cahill R, Noguchi A & Sly W S 2006 Enhancement of drug delivery to bone: Characterization of human tissue-non specific alkaline phosphatase tagged with an acidic oligopeptide. Molecular Genetics and Metabolism 88 244-255.
30. Nosjean O, Koyama I, Goseki M, Roux B & Komoda T 1997 Human tissue non-specific alkaline phosphatases: Sugar-moiety-induced enzymic and antigenic modulations and genetic aspects. Biochemical Journal 321 297-303.
31. Oda et al. 1999 J. Biochem 126: 694-699.
32. Rezende L A, Ciancaglini P, Pizauro J M & Leone F A 1998 Inorganic pyrophosphate-phosphohydrolytic activity associated with rat osseous plate alkaline phosphatase. Cellular and Molecular Biology 44 293-302.
33. Urlaub G, Kas E, Carothers A M & Chasin L A 1983 Deletion of the Diploid Dihydrofolate-Reductase Locus from Cultured Mammalian-Cells. Cell 33 405-412.
34. Waymire K G, Mahuren J D, Jaje J M, Guilarte T R, Coburn S P & MacGregor G R 1995 Mice Lacking Tissue Nonspecific Alkaline-Phosphatase Die from Seizures Due to Defective Metabolism of Vitamin-B-6. Nature Genetics 11 45-51.
35. Weiss, M. J. et al. 1988 A missense mutation in the human liver/bone/kidney alkaline phosphatase gene causing a lethal form of hypophosphatasia. Proc Natl Acad Sci USA 85, 7666-9.
36. Weninger M, Stinson R A, Plenk H, Bock P & Pollak A 1989 Biochemical and Morphological Effects of Human Hepatic Alkaline-Phosphatase in A Neonate with Hypophosphatasia. Acta Paediatrica Scandinavica 154-160.
37. Whyte M P 1994 Hypophosphatasia and the Role of Alkaline-Phosphatase in Skeletal Mineralization. Endocrine Reviews 15 439-461.
38. Whyte M. P. 1995 Alkaline Phosphatase: Placental and Tissue-nonspecific Isoenzymes hydrolyze Phosphoethanolamine, Inorganic Pyrophosphate, and Pyridoxal 5′-phosphate J. Clin. Invest. 95:1440-1445.
39. Whyte M P 2001 Hypophosphatasia. In The Metabolic and Molecular Bases of Disease, edn 8th, pp 5313-5329. Eds C L Scriver, A L Beaudet, W S Sly, D Valle & B Vogelstein. New York: McGraw-Hill Book Company.
40. Whyte M P 2002 Hypophosphatasia. Nature's window on alkaline phosphatase function in man. In Principle of Bone Biology, edn Second, pp 1229-1248. Eds J P Bilezikian, L G Raisz & G A Rodan. London: Academic Press.
41. Whyte M P, Kurtzberg J, McAlister W H, Mumm S, Podgornik M N, Coburn S P, Ryan L M, Miller C R, Gottesman G S, Smith A K, Douville J, Waters P, Armstrong R D & Martin P L 2003 Marrow cell transplantation for infantile hypophosphatasia. J Bone Miner Res 18 624-636.
42. Whyte M P, Mahuren J D, Vrabel L A & Coburn S P 1985 Markedly Increased Circulating Pyridoxal-5′-Phosphate Levels in Hypophosphatasia—Alkaline-Phosphatase Acts in Vitamin-B6 Metabolism. Journal of Clinical Investigation 76 752-756.
43. Whyte M P, McAlister W H, Patton L S, Magill H L, Fallon M D, Lorentz W B & Herrod H G 1984 Enzyme replacement therapy for infantile hypophosphatasia attempted by intravenous infusions of alkaline phosphatase-rich Paget plasma: results in three additional patients. J Pediatr 105 926-933.
44. Whyte M P, Valdes R, Ryan L M & McAlister W H 1982 Infantile hypophosphatasia: enzyme replacement therapy by intravenous infusion of alkaline phosphatase-rich plasma from patients with Paget bone disease. J Pediatr 101 379-386.
45. Zurutuza L, Muller F, Gibrat J F, Taillandier A, Simon-Bouy B, Serre J L & Mornet E 1999 Correlations of genotype and phenotype in hypophosphatasia. Human Molecular Genetics 8 1039-1046.
46. Beertsen W., Van den Bos T, Everts V, 1999, Root development in mice lacking functional tissue non-specific alkaline phosphatase gene: Inhibition of acellular cementum formation. J. Dent. Res. 78:1221-1229.

1. A bone targeted alkaline phosphatase comprising a polypeptide having the structure:
Z-sALP-Y-spacer-X-Wₙ-V,
wherein sALP is the extracellular domain of the alkaline phosphatase;
wherein V is absent or is an amino acid sequence of at least one amino acid;
X is absent or is an amino acid sequence of at least one amino acid;
Y is absent or is an amino acid sequence of at least one amino acid;
Z is absent or is an amino acid sequence of at least one amino acid; and
Wₙ is a polyaspartate or a polyglutamate wherein n=10 to 16.

2. The alkaline phosphatase of claim 1, wherein the sALP comprises amino acid residues 23-508 of SEQ ID NO: 15.

3. The alkaline phosphatase of claim 2, wherein the sALP consists of amino acid residues 23-512 of SEQ ID NO: 15.

4. The alkaline phosphatase of claim 2, wherein the sALP comprises amino acid residues 23-508 of SEQ ID NO: 18.

5. The alkaline phosphatase of claim 2, wherein the sALP consists of amino acid residues 23-512 of SEQ ID NO: 18.

6. The alkaline phosphatase of claim 1, wherein the sALP comprises amino acid residues 18-498 of SEQ ID NO: 16.

7. The alkaline phosphatase of claim 6, wherein the sALP consists of amino acid residues 18-502 of SEQ ID NO: 16.

8. The alkaline phosphatase of claim 6, wherein the sALP comprises amino acid residues 18-498 of SEQ ID NO: 19.

9. The alkaline phosphatase of claim 8, wherein the sALP consists of amino acid residues 18-502 of SEQ ID NO: 19.

10. (canceled)

11. (canceled)

12. The alkaline phosphatase of claim 8, wherein the sALP comprises amino acid residues 18-498 of SEQ ID NO: 8.

13. The alkaline phosphatase of claim 12, wherein the sALP consists of amino acid residues 18-502 of SEQ ID NO: 8.

14. The alkaline phosphatase of claim 1, wherein the spacer comprises a fragment crystallizable region (Fc).

15. The alkaline phosphatase of claim 14, wherein the Fc comprises a CH2 domain, a CH3 domain and a hinge region.

16. The alkaline phosphatase of claim 14, wherein the Fc is a constant domain of an immunoglobulin selected from the group consisting of IgG-1, IgG-2, IgG-3, IgG-3 and IgG-4.

17. The alkaline phosphatase of claim 16, wherein the Fc is a constant domain of an immunoglobulin IgG-1.

18. The alkaline phosphatase of claim 17, wherein the Fc is as set forth in SEQ ID NO: 3.

19. The alkaline phosphatase of claim 1, wherein Wₙ is a polyaspartate.

20. The alkaline phosphatase of claim 1, wherein n=10.

21. The alkaline phosphatase of claim 1, wherein Z is absent.

22. The alkaline phosphatase of claim 1, wherein Y is two amino acid residues.

23. The alkaline phosphatase of claim 22, wherein Y is leucine-lysine.

24. The alkaline phosphatase of claim 1, wherein X is two amino acid residues.

25. The alkaline phosphatase of claim 24, wherein X is aspartate-isoleucine.

26. The alkaline phosphatase of claim 1, wherein V is absent.

27. The alkaline phosphatase of claim 1, wherein the polypeptide is as set forth in SEQ ID NO: 4.

28. The alkaline phosphatase of claim 1, comprising the polypeptide in a form comprising a dimer.

29. (canceled)

30. The alkaline phosphatase of claim 1, in a pharmaceutically acceptable carrier.

31. The alkaline phosphatase of claim 30, wherein the pharmaceutically acceptable carrier comprises saline.

32. The alkaline phosphatase of claim 31, in a lyophilized form.

33. The alkaline phosphatase of claim 30, in a daily dosage of about 0.2 to about 20 mg/kg.

34. The alkaline phosphatase of claim 30, in a dosage of about 0.6 to about 60 mg/kg for administration every three days.

35. The alkaline phosphatase of claim 30, in a weekly dosage of about 1.4 to about 140 mg/kg.

36. The alkaline phosphatase of claim 30, in a weekly dosage of about 0.5 mg/kg.

37. An isolated nucleic acid comprising a sequence that encodes the polypeptide defined in claim 1.

38. An isolated nucleic acid consisting of a sequence that encodes the polypeptide defined in claim 1.

39. (canceled)

40. A recombinant expression vector comprising the nucleic acid of claim 1.

41. A recombinant adeno-associated virus vector comprising the nucleic acid of claim 37.

42. An isolated recombinant host cell transformed or transfected with the vector of claim 40.

43. A method of producing the alkaline phosphatase of claim 1, comprising culturing the host cell of claim 42, under conditions suitable to effect expression of the alkaline phosphatase and recovering the alkaline phosphatase from the culture medium.

44. The method of claim 43, wherein the host cell is a L cell, C127 cell, 3T3 cell, CHO cell, BHK cell, COS-7 cell or a Chinese Hamster Ovary (CHO) cell.

45. The method of claim 44, wherein the host cell is a Chinese Hamster Ovary (CHO) cell.

46. The method of claim 45, wherein the host cell is a CHO-DG44 cell.

47. A kit comprising the alkaline phosphatase defined in claim 1, and instructions to administer the polypeptide to a subject to correct or prevent a hypophosphatasia (HPP) phenotype.

48. A kit comprising the alkaline phosphatase defined in claim 1, and instructions to administer the polypeptide to a subject to correct or prevent aplasia, hypoplasia or dysplasia of dental cementum.

49. A method of using the alkaline phosphatase of claim 1, for correcting or preventing at least one hypophosphatasia (HPP) phenotype, comprising administering a therapeutically effective amount of the alkaline phosphatase to a subject in need thereof, whereby the at least one HPP phenotype is corrected or prevented in the subject.

50. The method of claim 49, wherein the subject has at least one HPP phenotype.

51. The method of claim 49, wherein the subject is likely to develop at least one HPP phenotype.

52. The method of claim 49, wherein the at least one HPP phenotype comprises HPP-related seizure.

53. The method of claim 49, wherein the at least one HPP phenotype comprises premature loss of deciduous teeth.

54. The method of claim 49, wherein the at least one HPP phenotype comprises incomplete bone mineralization.

55. The method of claim 54, wherein incomplete bone mineralization is incomplete femoral bone mineralization.

56. The method of claim 54 wherein incomplete bone mineralization is incomplete tibial bone mineralization.

57. The method of claim 54, wherein incomplete bone mineralization is incomplete metatarsal bone mineralization.

58. The method of claim 54, wherein incomplete bone mineralization is incomplete ribs bone mineralization.

59. The method of claim 49, wherein the at least one HPP phenotype comprises elevated blood and/or urine levels of inorganic pyrophosphate (PPᵢ).

60. The method of claim 49, wherein the at least one HPP phenotype comprises elevated blood and/or urine levels of phosphoethanolamine (PEA).

61. The method of claim 49, wherein the at least one HPP phenotype comprises elevated blood and/or urine levels of pyridoxal 5′-phosphate (PLP).

62. The method of claim 49, wherein the at least one HPP phenotype comprises inadequate weight gain.

63. The method of claim 49, wherein the at least one HPP phenotype comprises rickets.

64. The method of claim 49, wherein the at least one HPP phenotype comprises bone pain.

65. The method of claim 49, wherein the at least one HPP phenotype comprises calcium pyrophosphate dihydrate crystal deposition.

66. The method of claim 49, wherein the at least one HPP phenotype comprises aplasia, hypoplasia or dysplasia of dental cementum.

67. The method of claim 49, wherein the subject in need thereof has infantile HPP.

68. The method of claim 49, wherein the subject in need thereof has childhood HPP.

69. The method of claim 49, wherein the subject in need thereof has perinatal HPP.

70. The method of claim 49, wherein the subject in need thereof has adult HPP.

71. The method of claim 49, wherein the subject in need thereof has odontohypophosphatasia HPP.

72. A method of using the alkaline phosphatase of claim 1, for correcting or preventing aplasia, hypoplasia or dysplasia of dental cementum, comprising administering a therapeutically effective amount of the alkaline phosphatase to a subject in need thereof, whereby aplasia, hypoplasia or dysplasia of dental cementum is corrected or prevented in the subject.

73. The method of claim 49, wherein the administering comprises transfecting a cell in the subject with a nucleic acid encoding the alkaline phosphatase.

74. The method of claim 73, wherein the transfecting the cell is performed in vitro such that the alkaline phosphatase is expressed and secreted in an active form and administered to the subject with said cell.

75. The method of claim 49, wherein the administering comprises subcutaneous administration of the alkaline phosphatase to the subject.

76. The method of claim 49, wherein the administering comprises intravenous administration of the alkaline phosphatase to the subject.

77.-81. (canceled)
